(19) United States
(12) Patent Application Publication
(10) Pub. No.: US 2010/0190829 A1
(43) Pub. Date: Jul. 29, 2010

(54) RENIN INHIBITORS

(75) Inventors: John J. Baldwin, Gwynedd Valley, PA (US); Salvacion Cacatian, Blue Bell, PA (US); David A. Claremon, Maple Glen, PA (US); Lawrence W. Dillard, Yardley, PA (US); Patrick T. Flaherty, Pittsburgh, PA (US); Alexey V. Ishchenko, Somerville, MA (US); Lanqi Jia, Horsham, PA (US); Gerard McGeehan, Garnet Valley, PA (US); Robert D. Simpson, Wilmington, DE (US); Suresh B. Singh, Kendall Park, NJ (US); Colin M. Tice, Ambler, PA (US); Zhenrong Xu, Horsham, PA (US); Jing Yuan, Lansdale, PA (US); Wei Zhao, Eagleville, PA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Vitae Pharmaceuticals Inc

(21) Appl. No.: 12/665,198

(22) PCT Filed: Jun. 20, 2008
(86) PCT No.: PCT/US08/07700

§ 371 Date: Dec. 17, 2009

Related U.S. Application Data
(60) Provisional application No. 60/936,482, filed on Jun. 20, 2007.

(51) Int. Cl. A61K 31/445; C07D 211/56; C07C 271/20; A61K 31/27; A61P 9/12; A61P 9/04; A61P 27/06; A61P 5/46; A61P 25/22

(52) U.S. Cl. 514/329; 546/223; 560/27; 514/485

(57) ABSTRACT

Described are compounds that bind to aspartic proteases to inhibit their activity. They are useful in the treatment or amelioration of diseases associated with aspartic protease activity. Also described are methods of use of the compounds described herein in ameliorating or treating aspartic protease related disorders in a subject in need thereof.

### RELATED APPLICATION

This application claims the benefit of U.S. Provisional Application No. 60/936,482, filed Jun. 20, 2007. The entire teachings of the above application are incorporated herein by reference.

### BACKGROUND OF THE INVENTION

Aspartic proteases, including renin, β-secretase (BACE), Candida albicans secreted aspartyl proteases, HIV protease, HTLV protease and plasmepsins I and II, are implicated in a number of disease states. In hypertension elevated levels of angiotensin I, the product of renin catalyzed cleavage of angiotensinogen are present. Elevated levels of β-amyloid, the product of BACE activity on amyloid precursor protein, are widely believed to be responsible for the amyloid plaques present in the brains of Alzheimer's disease patients. Secreted aspartyl proteases play a role in the virulence of the pathogen Candida albicans. The viruses HIV and HTLV depend on their respective aspartic proteases for viral maturation. Plasmodium falciparum uses plasmepsins I and II to degrade hemoglobin.

In the renin-angiotensin-aldosterone system (RAAS) the biologically active peptide angiotensin II (Ang II) is generated by a two-step mechanism. The highly specific aspartic protease renin cleaves angiotensinogen to angiotensin I (Ang I), which is then further processed to Ang II by the less specific angiotensin-converting enzyme (ACE). Ang II is known to work on at least two receptor subtypes called AT₁ and AT₂. Whereas AT₁ seems to transmit most of the known functions of Ang II, the role of AT₂ is still unknown.

Modulation of the RAAS represents a major advance in the treatment of cardiovascular diseases (Zaman, M. A. et al Nature Reviews Drug Discovery 2002, 1, 621-636). ACE inhibitors and AT₁ blockers have been accepted as treatments of hypertension (Waeber B. et al., “The renin-angiotensin system: role in experimental and human hypertension”, in Berkenhager W. H., Reid J. L. (eds): Hypertension, and human hypertension”, in Berkenhager W. H., Reid J. L. (eds): Hypertension, Amsterdam, Elsevier Science Publishing Co, 1996, 489-519; Weber M. A., Am. J. Hypertens., 1992, 5, 247S). In addition, ACE inhibitors are used for renal protection (Rosenberg M. E. et al., Kidney International, 1994, 45, 403; Breyer J. A. et al., Kidney International, 1994, 45, S156), in the prevention of congestive heart failure (Vaughan D. E. et al., Cardiovasc. Res., 1994, 28, 159; Fouad-Tarazi F. et al., Am. J. Med., 1988, 84 (Suppl. 3A), 83) and myocardial infarction (Pfeffer M. A. et al., N Engl. J. Med, 1992, 327, 669).

Interest in the development of renin inhibitors stems from the specificity of renin (Kleinert H. D., Cardiovasc. Drugs, 1995, 9, 645). The only substrate known for renin is angiotensinogen, which can only be processed (under physiological conditions) by renin. In contrast, ACE can also cleave bradykinin besides Ang I and can be bypassed by chymase, a serine protease (Husain A., J. Hypertens., 1993, 11, 1155). In patients, inhibition of ACE thus leads to bradykinin accumulation causing cough (5-20%) and potentially life-threatening angioneurotic edema (0.1-0.2%) (Israili Z. H. et al., Annals of Internal Medicine, 1992, 117, 234). Chymase is not inhibited by ACE inhibitors. Therefore, the formation of Ang II is still possible in patients treated with ACE inhibitors. Blockade of the ATI receptor (e.g., by losartan) on the other hand overexposes other AT-receptor subtypes to Ang II, whose concentration is dramatically increased by the blockade of AT1 receptors.

In summary, renin inhibitors are not only expected to be superior to ACE inhibitors and AT₁ blockers with regard to safety, but more importantly also with regard to their efficacy in blocking the RAAS.

Only limited clinical experience (Azizi M. et al., J. Hypertens., 1994, 12, 419; Neutel J. M. et al., Am. Heart, 1991, 122, 1094) has been generated with renin inhibitors because their peptidomimetic character imparts insufficient oral activity (Kleinert H. D., Cardiovasc. Drugs, 1995, 9, 645). The clinical development of several compounds has been stopped because of this problem together with the high cost of goods. It appears as though only one compound has entered clinical trials (Rahuel J. et al., Chem. Biol., 2000, 7, 493; Mealy N. E., Drugs of the Future, 2001, 26, 1139). Thus, metabolically stable, orally bioavailable and sufficiently soluble renin inhibitors that can be prepared on a large scale are not available. Recently, the first non-peptide renin inhibitors were described which show high in vitro activity (Oefner C. et al., Chem. Biol., 1999, 6, 127; Patent Application WO 97/09311; Maerki H. P. et al., II Farmaco, 2001, 56, 21). The present invention relates to the unexpected identification of renin inhibitors of a non-peptidic nature and of low molecular weight. Orally active renin inhibitors which are active in indications beyond blood pressure regulation where the tissular renin-chymase system may be activated leading to pathophysiologically altered local functions such as renal, cardiac and vascular remodeling, atherosclerosis, and restenosis, are described.

All documents cited herein are incorporated by reference.

### SUMMARY OF THE INVENTION

Compounds have now been found that bind to aspartic proteases to inhibit their activity. Such compounds are useful in the treatment or amelioration of diseases associated with aspartic protease activity.

One embodiment of the invention is a compound represented by Structural Formula (I):

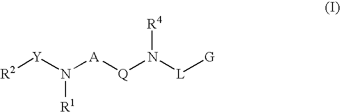

wherein:

R¹ is (C₃-C₇)cycloalkyl, phenyl, heteroaryl, or bicyclic heteroaryl each optionally substituted with 1 to 3 groups independently selected from: fluorine, chlorine, bromine, cyano, nitro, hydroxyl, (C₁-C₆)alkyl, (C₃-C₆)cycloalkyl, (C₄-C₇)cycloalkylalkyl, (C₂-C₆)alkenyl, (C₅-C₇)cycloalkylalkenyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, halo(C₂-C₆)alkenyl, halo(C₃-C₆)alkynyl, halo(C₅-C₇)-cycloalkylalkynyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy and (C₁-C₆)alkanesulfonyl; and phenyl, heteroaryl, phenoxy, heteroaryloxy, phenylthio, heteroarylthio, benzyl, heteroarylmethyl, benzyloxy and heteroarylmethoxy, each optionally substituted with 1 to 3 groups independently selected from: fluorine, chlorine, bromine, cyano, nitro, hydroxyl, (C₁-C₃)alkyl, halo(C₁-C₃)alkyl, (C₁-C₃)-alkoxy, and halo(C₁-C₃)alkoxy, and aminocarbonyl;

R² is (C₁-C₁₂)alkyl, (C₂-C₁₂)alkenyl, (C₂-C₁₂)alkynyl, (C₃-C₇)cycloalkyl, R^{2a}—O—, R^{2d}—O—, R^{2a}—S—, R^{2a}—O—Y₁—, R^{2a}—S—Y₁—, R^{2a}—O—Y₁—O—, R^{2a}—O—Y₁—S—, R^{2a}—S—Y₁—O—, R^{2a}—S—Y₁—S—, R^{2a}—O—Y₁—O—Y₁—, NH₂—C(O)—NH—Y₁—, NH₂—C(O)—NH—Y₁—O—, NH₂—C(O)—NH—Y₁—S—, R^{2a}—C(O)—NH—Y₁—, R^{2a}—C(O)—NH—Y₁—O—, R^{2a}—C(O)—NH—Y₁—S—, NH₂—S(O)₂—NH—Y₁—, NH₂—S(O)₂—NH—Y₁—O—, NH₂—S(O)₂—NH—Y₁—S—, R^{2a}—S(O)₂—NH—Y₁—, R^{2a}—S(O)₂—NH—Y₁—O—, R^{2a}—S(O)₂—NH—Y₁—S—, H—C(O)—NH—, H—C(O)—NH—Y₁—, H—C(O)—NH—Y₁—O—, H—C(O)—NH—Y₁—S—, R^{2a}—O—C(O)—NH—, R^{2a}—O—C(S)—NH—, R^{2a}—O—C(O)—NH—Y₁—, R^{2a}—O—C(O)—NH—Y₁—O—, R^{2a}—O(O)—NH—Y₁—S—, R^{2a}—NH—C(O)—NH—Y₁—, (R^{2a})₂—N—C(O)—NH—Y₁—, R^{2a}—NH—C(O)—NH—Y₁—O—, (R^{2a})₂—N—C(O)—NH—Y₁—O—, R^{2a}—NH—C(O)—NH—Y₁—S—, (R^{2a})₂—N—C(O)—NH—Y₁—S—, NH₂—C(O)—, NH₂—C(S)—, NH₂—C(O)—Y₁—, NH₂—C(O)—Y₁—O—, NH₂—C(O)—Y₁—S—, R^{2a}—NH—C(O)—, R^{2a}—NH—C(S)—, R^{2a}—NH—C(O)—Y₁—, R^{2a}—NH—C(O)—Y₁—O—, R^{2a}—NH—C(O)—Y₁—S—, NH₂—C(O)—O—, NH₂—C(S)—O—, NH₂—C(O)—O—Y₁, NH₂—C(O)—O—Y₁—O—, NH₂—C(O)—O—Y₁—S—, R^{2a}—NH—C(O)—O—, R^{2a}—NH—C(S)—O—, R^{2a}—NH—C(O)—O—Y₁—, R^{2a}—NH—C(O)—O—Y₁—O—, R^{2a}—NH—C(O)—O—Y₁—S—, R^{2a}—NH—C(O)—NH—, R^{2a}—C(O)—NH—, NH₂—C(S)—S—, NH₂—C(O)—S—, R^{2a}—NH—C(S)—S—, R^{2a}—NH—C(O)—S—, R^{2a}—S—C(S)—NH—, R^{2a}—S—C(O)—NH—, R^{2a}—C(O)—, R^{2a}—C(S)—, NHC(═NR^{2e})(NH₂), —NHC(═NR^{2e})(NHR^{2a})

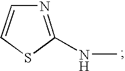

R^{2a} is straight or branched (C₁-C₁₂)alkyl, straight or branched (C₁-C₁₂)haloalkyl, (C₃-C₇)cycloalkyl, or straight or branched C₁-C₁₂ alkoxyalkyl, R^{2d}═(C₂-C₁₂)alkenyl, and R^{2e} is H, (C₁-C₆)alkyl, phenyl, heteroaryl, cyano, nitro, —S(O)R^{2a}, —S(O)₂R^{2a}, —S(O)₂NHR^{2a}, —S(O)₂NR^{2a}R^{2a}, —C(O)R^{2a}, —C(S)R^{2a}, —C(O)OR^{2a}, —C(S)OR^{2a}, —C(O)(NH₂), —C(O)(NHR^{2a});

Y₁ is a straight or branched C₁-C₁₂ alkylene optionally substituted with one or more halogens and optionally interrupted at an internal carbon atom with oxygen;

R² can be substituted by at least one of:
a) 1 to 6 halogen atoms; or
b) one substituent selected from the group consisting of cyano, hydroxyl, (C₁-C₃)alkoxy, (C₃-C₆)cycloalkyl, (C₃-C₆)cycloalkoxy, halo(C₁-C₃)alkoxy, halo(C₃-C₆)cycloalkyl and halo(C₃-C₆)cycloalkoxy; and

wherein the thio-moiety of said unsubstituted or substituted R^{2a}—S—, R^{2a}—S—Y₁, R^{2a}—O—Y₁—S—, R^{2a}—S—Y₁—O—, R^{2a}—S—Y₁—S—, NH₂—C(O)—NH—Y₁—S—, R^{2a}—C(O)—NH—Y₁—S—, NH₂—S(O)₂—NH—Y₁—S—, R^{2a}—S(O)₂—NH—Y₁—S—, H—C(O)—NH—Y₁—S—, R^{2a}—O—C(O)—NH—Y₁—S—, R^{2a}—NH—C(O)—NH—Y₁—S—, (R^{2a})₂—N—C(O)—NH—Y₁—S—, NH₂—C(O)—Y₁—S—, R^{2a}—NH—C(O)—Y₁—S—, NH₂—C(O)—O—Y₁—S—, R^{2a}—NH—C(O)—O—Y₁—S—, NH₂—C(S)—S—, NH₂—C(O)—S—, R^{2a}—NH—C(S)S—, R^{2a}—NH—C(O)—S—, R^{2a}—S—C(S)—NH—, R^{2a}—S—C(O)—NH—, and

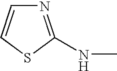

is optionally replaced by —S(O)— or —S(O)₂—; and

wherein the carbonyl moiety of said unsubstituted or substituted NH₂—C(O)—NH—Y₁—, NH₂—C(O)—NH—Y₁—O—, NH₂—C(O)—NH—Y₁—S—, R^{2a}—C(O)—NH—Y₁—, R^{2a}—C(O)—NH—Y₁—O—, R^{2a}—C(O)—NH—Y₁—S—, H—C(O)—NH—, H—C(O)—NH—Y₁—, H—C(O)—NH—Y₁—O—, H—C(O)—NH—Y₁—S—, R^{2a}—O—C(O)—NH—, R^{2a}—O—C(O)—NH—Y₁—, R^{2a}—O—C(O)—NH—Y₁—O—, R^{2a}—O—C(O)—NH—Y₁—S—, R^{2a}—NH—C(O)—NH—Y₁—, (R^{2a})₂—N—C(O)—NH—Y₁—, R^{2a}—NH—C(O)—NH—Y₁—O—, (R^{2a})₂—N—C(O)—NH—Y₁—, R^{2a}—NH—C(O)—NH—Y₁—S—, (R^{2a})₂—N—C(O)—NH—Y₁—S—, NH₂—C(O)—, NH₂—C(O)—Y₁—, NH₂—C(O)—Y₁—O—, NH₂—C(O)—Y₁—S—, R^{2a}—NH—C(O)—, R^{2a}—NH—C(O)—Y₁—, R^{2a}—NH—C(O)—Y₁—O—, R^{2a}—NH—C(O)—Y₁—S—, NH₂—C(O)—O—, NH₂—C(O)—O—Y₁—, NH₂—C(O)—O—Y₁—O—, NH₂—C(O)—O—Y₁—S—, R^{2a}—NH—C(O)—O—, R^{2a}—NH—C(O)—O—Y₁—, R^{2a}—NH—C(O)—O—Y₁—O—, R^{2a}—NH—C(O)—O—Y₁—S—, R^{2a}—NH—C(O)—NH—, R^{2a}—C(O)—NH—, NH₂—C(O)—S—, R^{2a}—NH—C(O)—S—, R^{2a}—S—C(O)—NH—, and R^{2a}—C(O)—

is optionally replaced by a thiocarbonyl moiety;

provided that when R₂ is attached to

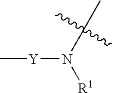

through a heteroatom, Y can not be a covalent bond;

A is a saturated or unsaturated 4-, 5-, 6-, or 7-membered ring which is optionally bridged by (CH₂)ₚ via bonds to two members of said ring, wherein said ring is composed of carbon atoms and 0-2 hetero atoms selected from the group consisting of 0, 1, or 2 nitrogen atoms, 0 or 1 oxygen atoms, and 0 or 1 sulfur atoms, said ring being optionally and independently substituted with zero to four halogen atoms, (C₁-C₆)alkyl groups, halo(C₁-C₆)alkyl groups or oxo groups such that when there is substitution with one oxo group on a carbon atom it forms a carbonyl group, and when there is substitution of one or two oxo groups on sulfur it forms sulfoxide or sulfone groups, respectively;

p is 1 to 3;

Y is a covalent bond or C₁-C₁₀ alkylene, C₁-C₁₀ alkenylene or C₁-C₁₀ alkynylene, each optionally substituted at one or more substitutable carbon atoms with halogen, cyano, hydroxyl, (C₁-C₃)alkyl, (C₁-C₃)alkoxy or halo(C₁-C₃)alkoxy,

Q is Q1, Q2, Q3, Q4, Q5, or Q6:

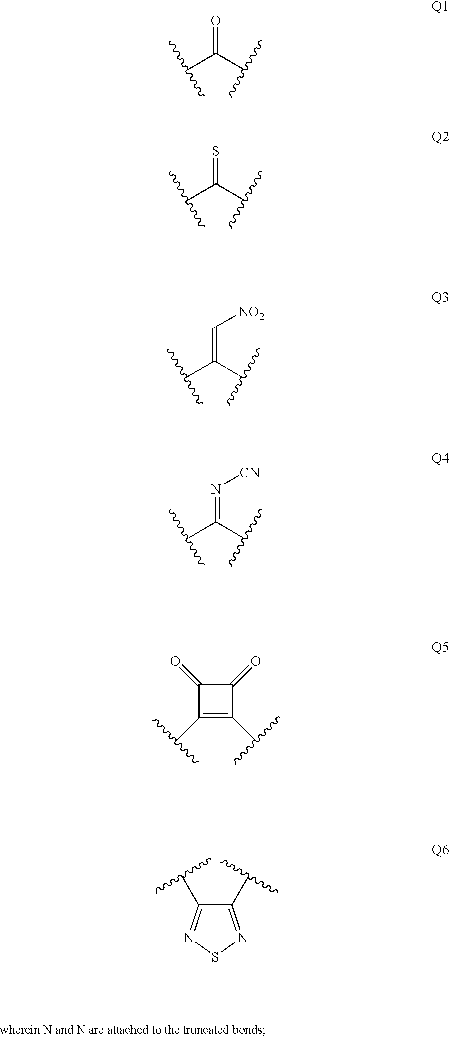

R⁴ is H, (C₁-C₆)alkyl, halo(C₁-C₆)alkyl; (C₁-C₃)alkoxy(C₁-C₃)alkyl, or cyano(C₁-C₆)alkyl;

G is OH, OR^{e}, NH₂, NHR^{e}, NR^{e}R^{f}, C(═NH)NH₂, C(═NH)NHR^{e}, NHC(═NH)NH₂, or NHC(═NH)NHR^{e};

L is 1) a linear (C₂-C₄)alkyl chain when G is OH, OR^{e}, NH₂, NHR^{e}, NR^{e}R^{f}, NHC(═NH)NH₂, or NHC(═NH)NHR^{e}, or 2) a linear (C₁-C₃)alkyl chain when G is C(═NH)NH₂ or C(═NH)NHR^{e};

L is optionally substituted by 1-4 groups independently selected from R⁵, R^{5a}, R⁶, and R^{6a}; one or more of the carbon atoms of L may be part of a 3-, 4-, 5-, 6-, or 7-membered saturated ring composed of carbon atoms, and 0-2 hetero atoms selected from 0 or 1 nitrogen atoms, 0 or 1 oxygen atoms, and 0 or 1 sulfur atoms; said saturated ring being optionally substituted with up to four groups selected from halogen, (C₁-C₆)alkyl, halo(C₁-C₆)alkyl, (C₃-C₆)cycloalkyl, halo(C₃-C₆)cycloalkyl, (C₄-C₇)cycloalkylalkyl, halo(C₄-C₇)cycloalkylalkyl, and oxo, such that when there is substitution with one oxo group on a carbon atom it forms a carbonyl group and when there is substitution of one or two oxo groups on sulfur it forms sulfoxide or sulfone groups, respectively);

R⁵, R^{5a}, R⁶, and R^{6a} is each independently selected from 1) H, (C₁-C₁₂)alkyl, halo(C₁-C₁₂)alkyl, hydroxy(C₁-C₁₂)alkyl, (C₃-C₁₀)cycloalkyl, (C₃-C₁₀)cycloalkyl, (C₃-C₁₀)cycloalkylalkyl, halo(C₃-C₁₀)cycloalkylalkyl, hydroxy(C₃-C₁₀)cycloalkylalkyl, (C₁-C₂)alkyl(C₃-C₁₀)cycloalkylalkyl, halo(C₁-C₂)alkyl(C₃-C₁₀)cycloalkylalkyl, di(C₁-C₂)alkyl(C₃-C₁₀)cycloalkylalkyl, hydroxy(C₁-C₂)alkyl (C₃-C₁₀)cycloalkylalkyl, hydroxy di(C₁-C₂)alkyl(C₃-C₁₀)cycloalkylalkyl, (C₂-C₁₂)alkenyl, (C₅-C₈)cycloalkyl(C₁-C₃)alkenyl, (C₂-C₁₂)alkynyl, (C₃-C₈)cycloalkyl(C₁-C₃)alkynyl, (C₄-C₁₂)bicycloalkyl(C₁-C₃)alkyl, (C₈-C₁₄)tricycloalkyl(C₁-C₃)alkyl, (C₁-C₆)alkoxy(C₁-C₆)alkyl, halo(C₁-C₆)alkoxy(C₁-C₆)alkyl, (C₃-C₈)cycloalkoxy(C₁-C₃)alkyl, (C₁-C₆)alkylthio(C₁-C₆)alkyl, halo(C₁-C₆)alkylthio(C₁-C₆)alkyl, (C₃-C₈)cycloalkylthio(C₁-C₃)alkyl, saturated heterocyclyl, and saturated heterocyclyl(C₁-C₃)alkyl wherein (a) hydrogen atoms in these groups are optionally substituted by 1 to 6 groups independently selected from halogen, cyano, nitro, hydroxy, (C₁-C₆)alkyl, (C₁-C₆)alkoxy, (C₃-C₆)cycloalkyl, (C₃-C₇)cycloalkylalkyl, halo(C₃-C₇)cycloalkylalkyl, (C₂-C₆)alkenyl, halo(C₂-C₆)alkenyl, (C₃-C₇)cycloalkylalkenyl, (C₂-C₆)alkynyl, halo(C₂-C₆)alkynyl, (C₃-C₇)cycloalkylalkoxy, halo(C₃-C₇)cycloalkylalkoxy, (C₃-C₇)cycloalkoxy, halo(C₁-C₆)alkyl, (C₃-C₇)cycloalkylalkynyl, halo(C₃-C₇)cycloalkylalkynyl, halo(C₁-C₆)alkoxy, halo(C₃-C₇)cycloalkyl, halo(C₃-C₇)cycloalkoxy, (C₁-C₆)alkylsulfonyl, aminocarbonyl and wherein (b) divalent sulfur atoms are optionally oxidized to sulfoxide or sulfone;

or 2) phenyl, naphthyl, heteroaryl, phenyl(C₁-C₃)alkyl, phenoxymethyl, naphthyl(C₁-C₃)alkyl, and heteroaryl(C₁-C₃)alkyl, each optionally substituted with 1 to 3 groups independently selected from: halogen (e.g., fluorine, chlorine, bromine, iodine), cyano, nitro, amino, hydroxy, carboxy, (C₁-C₆)alkyl, (C₃-C₆)cycloalkyl, (C₄-C₇)cycloalkylalkyl, (C₂-C₆)alkenyl, halo(C₂-C₆)alkenyl, (C₃-C₆)cycloalkylalkenyl, (C₂-C₆)alkynyl, halo(C₂-C₆)alkynyl, (C₃-C₆)cycloalkyl-(C₂-C₄)alkynyl, halo(C₃-C₇)cycloalkylalkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylhio, (C₄-C₇)cycloalkylalkylthio, halo(C₁-C₆)alkylthio, halo(C₃-C₆)cycloalkylhio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)-cycloalkylalkanesulfonyl, (C₁-C₆)alkylamino, di(C₁-C₆)alkylamino, (C₁-C₆)-alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)-cycloalkylalkoxy(C₁-C₆)alkyl, (C₁-C₈)alkylthio(C₁-C₆)alkyl, (C₃-C₈)cycloalkylthio(C₁-C₆)alkyl, (C₄-C₈)cycloalkylalkylthio(C₁-C₆)alkyl, halo(C₁-C₈)alkylthio(C₁-C₆)alkyl, halo(C₃-C₈)cycloalkylhio(C₁-C₆)alkyl, halo(C₄-C₈)-cycloalkylalkylthio(C₁-C₆)alkyl, (C₁-C₈)alkanesulfinyl(C₁-C₆)alkyl, (C₃-C₈)-cycloalkanesulfinyl(C₁-C₆)alkyl, (C₄-C₈)cycloalkyl-alkanesulfinyl(C₁-C₆)alkyl, halo(C₁-C₈)alkanesulfinyl(C₁-C₆)alkyl, halo(C₃-C₈)cycloalkanesulfinyl(C₁-C₆)alkyl, halo(C₄-C₈)cycloalkylalkanesulfinyl(C₁-C₆)alkyl, (C₁-C₈)alkane-sulfonyl(C₁-C₆)alkyl, (C₃-C₈)cycloalkanesulfonyl(C₁-C₆)alkyl, (C₄-C₈)cycloalkylalkanesulfonyl(C₁-C₆)alkyl, halo(C₁-C₈)alkanesulfonyl(C₁-C₆)alkyl, halo(C₃-C₈)cycloalkanesulfonyl(C₁-C₆)alkyl, halo(C₄-C₈)cycloalkylalkanesulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylamino-carbonyl(C₁-C₆)alkyl, di(C₁-C₈)alkylaminocarbonyl(C₁-C₆)alkyl and (C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylamino-carboxy(C₁-C₆)alkyl and di(C₁-C₈)alkylaminocarboxy(C₁-C₆)alkyl, phenyl, naphthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, naphthyl(C₁-C₃)alkyl, heteroaryl(C₁-C₃)alkyl, and bicyclic heteroaryl(C₁-C₃)alkyl, wherein the aromatic and heteroaromatic groups are optionally substituted with 1 to 3 groups independently selected from fluorine, chlorine, cyano, (C₁-C₃)alkyl, halo(C₁-C₃)alkyl, (C₁-C₃)alkoxy, halo(C₁-C₃)-alkoxy, (C₁-C₃)alkanesulfonyl, and (C₁-C₃)alkoxycarbonyl;

R^{e} is a) (C₁-C₁₂)alkyl, (C₄-C₁₂)cycloalkylalkyl, halo(C₁-C₁₂)alkyl, halo(C₄-C₂)cycloalkylalkyl, (C₂-C₁₂)alkenyl, (C₈-C₁₂)cycloalkylalkenyl, halo(C₂-C₁₂)alkenyl, halo(C₈-C₁₂)cycloalkylalkenyl, (C₂-C₁₂)alkynyl, (C₅-C₁₂)cycloalkylalkynyl, halo(C₂-C₁₂)alkynyl, halo(C₈-C₁₂)cycloalkylalkynyl, (C₁-C₆)alkoxy(C₁-C₆)alkyl, halo(C₁-C₆)alkoxy(C₁-C₆)alkyl, (C₁-C₆)alkylthio(C₁-C₆)alkyl, halo(C₁-C₆)alkylthio(C₁-C₆)alkyl, (C₁-C₆)alkanesulfinyl(C₁-C₆)alkyl, halo(C₁-C₆)alkane-sulfinyl(C₁-C₆)alkyl, (C₁-C₆)alkanesulfonyl(C₁-C₆)alkyl, halo(C₁-C₆)alkanesulfonyl(C₁-C₆)alkyl, aminocarbonyl(C₁-C₆)alkyl, (C₁-C₆)alkylaminocarbonyl(C₁-C₆)alkyl, di(C₁-C₆)alkylamino-carbonyl(C₁-C₆)alkyl, cyano(C₁-C₆)alkyl, carboxy(C₁-C₆)alkyl, (C₁-C₆)alkoxycarbonyl(C₁-C₆)alkyl, saturated heterocyclyl, or saturated heterocyclyl(C₁-C₆)alkyl or b) phenyl, naphthyl, heteroaryl, phenyl(C₁-C₃)alkyl, naphthyl(C₁-C₃)alkyl, or heteroaryl(C₁-C₃)alkyl, each of a) and b) is optionally substituted by 1 to 3 groups independently selected from:

1) fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)cycloalkyl-(C₂-C₄)alkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylhio, (C₄-C₇)cycloalkylalkylthio, halo(C₁-C₆)alkylthio, halo(C₃-C₆)cycloalkylhio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃-C₆)cycloalkane-sulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)-cycloalkylalkanesulfonyl, (C₁-C₆)alkylamino, di(C₁-C₆)alkylamino, (C₁-C₆)alkoxy-(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl and di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)-cycloalkylalkoxy(C₁-C₆)alkyl, (C₁-C₈)alkylthio(C₁-C₆)alkyl, (C₃-C₈)cycloalkylhio(C₁-C₆)alkyl, (C₄-C₈)cycloalkylalkylthio(C₁-C₆)alkyl, halo(C₁-C₈)alkylthio(C₁-C₆)alkyl, halo(C₃-C₈)cycloalkylhio(C₁-C₆)alkyl, halo(C₄-C₈)-cycloalkylalkylthio(C₁-C₆)alkyl, (C₁-C₈)alkanesulfinyl(C₁-C₆)alkyl, (C₃-C₈)-cycloalkanesulfinyl(C₁-C₆)alkyl, (C₄-C₈)cycloalkyl-alkanesulfinyl(C₁-C₆)alkyl, halo(C₁-C₈)alkanesulfinyl(C₁-C₆)alkyl, halo(C₃-C₈)cycloalkanesulfinyl(C₁-C₆)alkyl, halo(C₄-C₈)cycloalkylalkanesulfinyl(C₁-C₆)alkyl, (C₁-C₈)alkane-sulfonyl(C₁-C₆)alkyl, (C₃-C₈)cycloalkanesulfonyl(C₁-C₆)alkyl, (C₄-C₈)cycloalkylalkanesulfonyl(C₁-C₆)alkyl, halo(C₁-C₈)alkanesulfonyl(C₁-C₆)alkyl, halo(C₃-C₈)cycloalkanesulfonyl(C₁-C₆)alkyl, halo(C₄-C₈)cycloalkylalkanesulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylamino-carbonyl(C₁-C₆)alkyl, di(C₁-C₈)alkylaminocarbonyl(C₁-C₆)alkyl(C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylamino-carboxy(C₁-C₆)alkyl and di(C₁-C₈)alkylaminocarboxy(C₁-C₆)alkyl; or 2) phenyl, naphthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, naphthyl(C₁-C₃)alkyl, heteroaryl(C₁-C₃)alkyl, and bicyclic heteroaryl(C₁-C₃)alkyl, each optionally substituted with 1 to 3 groups independently selected from fluorine, chlorine, cyano, (C₁-C₃)alkyl, halo(C₁-C₃)alkyl, (C₁-C₃)alkoxy, halo(C₁-C₃)alkoxy, (C₁-C₃)alkanesulfonyl, and (C₁-C₃)-alkoxycarbonyl; or

b) R^{e} is a saturated divalent radical composed of carbon atoms, and 0, 1 or 2 hetero atoms selected from 0 or 1 nitrogen atoms, 0 or 1 oxygen atoms, and 0 or 1 sulfur atoms that is attached to any core carbon atom on L to form a saturated 3-, 4-, 5-, 6-, or 7-membered L-G ring; said L-G ring being optionally substituted with 1 to 4 groups selected from halogen, fluorine, (C₁-C₈)alkyl, halo(C₁-C₈)alkyl, (C₃-C₈)cycloalkyl, halo(C₃-C₈)cycloalkyl, hydroxy(C₃-C₈)cycloalkyl, (C₃-C₈)cycloalkyl(C₁-C₃)alkyl, halo(C₃-C₈)cycloalkyl(C₁-C₃)alkyl, hydroxy(C₃-C₈)cycloalkyl(C₁-C₃)alkyl, (C₁-C₈)alkoxy, halo(C₁-C₈)alkoxy, (C₃-C₈)cycloalkoxy, halo(C₃-C₈)cycloalkoxy, hydroxy(C₃-C₈)cycloalkoxy, (C₁-C₈)alkoxy(C₁-C₃)alkyl, halo(C₁-C₈)alkoxy(C₁-C₃)alkyl, (C₃-C₈)cycloalkoxy(C₁-C₃)alkyl, halo(C₃-C₈)cycloalkoxy(C₁-C₃)alkyl, hydroxy(C₃-C₈)cycloalkoxy(C₁-C₃)alkyl, (C₃-C₈)cycloalkyl(C₁-C₃)alkoxy(C₁-C₃)alkyl, halo(C₃-C₈)cycloalkyl(C₁-C₃)alkoxy(C₁-C₃)alkyl, hydroxy(C₃-C₈)cycloalkyl(C₁-C₃)alkoxy(C₁-C₃)alkyl, (C₁-C₈)alkylthio, halo(C₁-C₈)alkylthio, (C₃-C₈)cycloalkylthio, halo(C₃-C₈)cycloalkylthio, hydroxy(C₃-C₈)cycloalkylthio, (C₃-C₈)cycloalkyl(C₁-C₃)alkylthio, halo(C₃-C₈)cycloalkyl(C₁-C₃)alkylthio, hydroxy(C₃-C₈)cycloalkyl(C₁-C₃)alkylthio, (C₁-C₈)alkylthio(C₁-C₃)alkyl, halo(C₁-C₈)alkylthio(C₁-C₃)alkyl, (C₃-C₈)cycloalkylthio(C₁-C₃)alkyl, halo(C₃-C₈)cycloalkylthio(C₁-C₃)alkyl, hydroxy(C₃-C₈)cycloalkylthio(C₁-C₃)alkyl, (C₃-C₈)cycloalkyl(C₁-C₃)alkylthio(C₁-C₃)alkyl, halo(C₃-C₈)cycloalkyl(C₁-C₃)alkylthio(C₁-C₃)alkyl, hydroxy(C₃-C₈)cycloalkyl(C₁-C₃)alkylthio(C₁-C₃)alkyl, heterocyclyl, and oxo;

R^{f} is (C₁-C₆)alkyl or halo(C₁-C₆)alkyl; or

an enantiomer, diastereomer or pharmaceutically acceptable salt thereof.

In another embodiment, the present invention is directed to pharmaceutical compositions comprising a compound described herein or enantiomers, diastereomers, or salts thereof and a pharmaceutically acceptable carrier or excipient.

In another embodiment, the present invention is directed to a method of antagonizing aspartic protease inhibitors in a subject in need thereof comprising administering to the subject an effective amount of a compound described herein or an enantiomer, diastereomer, or salt thereof.

In another embodiment the present invention is directed to method for treating or ameliorating an aspartic protease mediated disorder in a subject in need thereof comprising administering to said subject an effective amount of a compound described herein or an enantiomer, diastereomer, or salt thereof.

In another embodiment, the present invention is directed to a method for treating or ameliorating a renin mediated disorder in a subject in need thereof comprising administering to the subject an effective amount of a compound described herein or an enantiomer, diastereomer, or salt thereof.

In another embodiment, the present invention is directed to a method for the treatment of hypertension in a subject in need thereof comprising administering to the subject a compound described herein in combination therapy with one or more additional agents said additional agent selected from the group consisting of α-blockers, β-blockers, calcium channel blockers, diuretics, angiotensin converting enzyme (ACE) inhibitors, dual ACE and neutral endopeptidase (NEP) inhibitors, angiotensin-receptor blockers (ARBs), aldosterone synthase inhibitors, aldosterone-receptor antagonists, and endothelin receptor antagonists.

### DETAILED DESCRIPTION OF THE INVENTION

The invention is directed to an aspartic protease inhibitor compounds represented by Structural Formula I or enantiomers, diastereomers or pharmaceutically acceptable salts thereof (i.e., pharmaceutically acceptable salts of the compounds, enantiomers and diastereomers). Values and particular values for the variables in Structural Formula I are provided in the following paragraphs. It is understood that the invention encompasses all combinations of the substituent variables (i.e., R¹, R², R³, etc.) defined herein. For Structural Formula I:

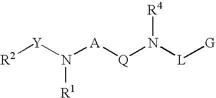

or a pharmaceutically acceptable salt thereof, wherein:

In one embodiment, R¹ is (C₃-C₇)cycloalkyl, phenyl, heteroaryl, or bicyclic heteroaryl each optionally substituted with 1 to 3 groups independently selected from: fluorine, chlorine, bromine, cyano, nitro, hydroxyl, (C₁-C₆)alkyl, (C₃-C₆)cycloalkyl, (C₄-C₇)cycloalkylalkyl, (C₂-C₆)alkenyl, (C₅-C₇)cycloalkylalkenyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, halo(C₂-C₆)alkenyl, halo(C₃-C₆)alkynyl, halo(C₅-C₇)-cycloalkylalkynyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy and (C₁-C₆)alkanesulfonyl; and phenyl, heteroaryl, phenoxy, heteroaryloxy, phenylthio, heteroarylthio, benzyl, heteroarylmethyl, benzyloxy and heteroarylmethoxy, each optionally substituted with 1 to 3 groups independently selected from: fluorine, chlorine, bromine, cyano, nitro, hydroxyl, (C₁-C₃)alkyl, halo(C₁-C₃)alkyl, (C₁-C₃)-alkoxy, and halo(C₁-C₃)alkoxy, and aminocarbonyl.

In another embodiment, R¹ is a phenyl optionally substituted with (R₁₁)ₙ, wherein n-0-3 and R¹¹ is independently selected from: fluorine, chlorine, bromine, cyano, nitro, (C₁-C₆)alkyl, (C₃-C₆)cycloalkyl, (C₄-C₇)cycloalkylalkyl, (C₂-C₆)alkenyl, (C₅-C₇)cycloalkylalkenyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, halo(C₂-C₆)alkenyl, halo(C₃-C₆)alkynyl, halo(C₅-C₇)-cycloalkylalkynyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy and (C₁-C₆)alkanesulfonyl; or 2) phenyl, heteroaryl, phenoxy, heteroaryloxy, phenylthio, heteroarylthio, benzyl, heteroarylmethyl, benzyloxy and heteroarylmethoxy, each optionally substituted with 1 to 3 groups independently selected from: fluorine, chlorine, cyano, (C₁-C₃)alkyl, halo(C₁-C₃)alkyl, (C₁-C₃)-alkoxy, and halo(C₁-C₃)alkoxy, and aminocarbonyl

In another particular embodiment, R¹ is phenyl optionally substituted with 1-3 groups independently selected from chloro, fluoro or methyl. In another particular embodiment, R¹ is phenyl substituted with chloro. In a most particular embodiment, R¹ is phenyl substituted with chloro at the carbon atom that is meta to the carbon atom that links phenyl to the rest of the molecule.

In one embodiment, R² is (C₁-C₁₂)alkyl, (C₂-C₁₂)alkenyl, (C₂-C₁₂)alkynyl, (C₃-C₇)cycloalkyl, R^{2a}—O—, R^{2d}—O—, R^{2a}—S—, R^{2a}—Y₁—, R^{2a}—S—Y₁—, R^{2a}—O—Y₁—O—, R^{2a}—O—Y₁—S—, R^{2a}—S—Y₁—O—, R^{2a}—S—Y₁—S—, R^{2a}—O—Y₁—O—Y₁—, NH₂—C(O)—NH—Y₁—, NH₂—C(O)—NH—Y₁—O—, NH₂—C(O)—NH—Y₁—S—, R^{2a}—C(O)—NH—Y₁—, R^{2a}—C(O)—NH—Y₁—O—, R^{2a}—C(O)—NH—Y₁—S—, NH₂—S(O)₂—NH—Y₁—, NH₂—S(O)₂—NH—Y₁—O—, NH₂—S(O)₂—NH—Y₁—S—, R^{2a}—S(O)₂—NH—Y₁—, R^{2a}—S(O)₂—NH—Y₁—O—, R^{2a}—S(O)₂—NH—Y₁—S—, H—C(O)—NH—, H—C(O)—NH—Y₁—, H—C(O)—NH—Y₁—O—, H—C(O)—NH—Y₁—S—, R^{2a}—O—C(O)—NH—, R^{2a}—O—C(S)—NH—, R^{2a}—O—C(O)—NH—Y₁—, R^{2a}—O—C(O)—NH—Y—O—, R^{2a}—O—C(O)—NH—Y —S—, R^{2a}—NH—C(O)—NH—Y₁—, (R^{2a})₂—N—C(O)—NH—Y₁—, R^{2a}—NH—C(O)—NH—Y₁—O—, (R^{2a})₂—N—C(O)—NH—Y₁—O—, R^{2a}—NH—C(O)—NH—Y₁—S—, (R^{2a})₂—N—C(O)—NH—Y₁—S—, NH₂—C(O)—, NH₂—C(S)—, NH₂—C(O)—Y₁—, NH₂—C(O)—Y₁—O—, NH₂—C(O)—Y₁—S—, R^{2a}—NH—C(O)—, R^{2a}—NH—C(S)—, R^{2a}—NH—C(O)—Y₁—, R^{2a}—NH—C(O)—Y₁—O—, R^{2a}—NH—C(O)—Y₁—S—, NH₂—C(O)—O—, NH₂—C(S)—O—, NH₂—C(O)—O—Y₁—, NH₂—C(O)—O—Y₁—O—, NH₂—C(O)—O—Y₁—S—, R^{2a}—NH—C(O)—O—, R^{2a}—NH—C(S)—O—, R^{2a}—NH—C(O)—O—Y₁—, R^{2a}—NH—C(O)—O—Y₁—O—, R^{2a}—NH—C(O)—O—Y₁—S—, R^{2a}—NH—C(O)—NH—, R^{2a}—C(O)—NH—, NH₂—C(S)—S—, NH₂—C(O)—S—, R^{2a}—NH—C(S)—S—, R^{2a}—NH—C(O)—S—, —R^{2a}—S—C(S)—NH—, R^{2a}S—C(O)—NH—, R^{2a}—C(O)—, R^{2a}—C(S)—, NHC(═NR^{2e})(NH₂), —NHC(═NR^{2e})(NHR^{2a}),

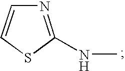

R² can be substituted by at least one of:
a) 1 to 6 halogen atoms; or
b) one substituent selected from the group consisting of cyano, hydroxyl, (C₁-C₃)alkoxy, (C₃-C₆)cycloalkyl, (C₃-C₆)cycloalkoxy, halo(C₁-C₃)alkoxy, halo(C₃-C₆)cycloalkyl and halo(C₃-C₆)cycloalkoxy; and

wherein the thio-moiety of said unsubstituted or substituted R^{2a}—S—, R^{2a}—S—Y₁, R^{2a}—O—Y₁—S—, R^{2a}—S—Y₁—O—, R^{2a}—S—Y₁—S—, NH₂—C(O)—NH—Y₁—S—, R^{2a}—C(O)—NH—Y₁—S—, NH₂—S(O)₂—NH—Y₁—S—, R^{2a}—S(O)₂—NH—Y₁—S—, H—C(O)—NH—Y₁—S—, R^{2a}—O—C(O)—NH—Y₁—S—, R^{2a}—NH—C(O)—NH—Y₁—S—, (R^{2a})₂—N—C(O)—NH—Y₁—S—, NH₂—C(O)—Y₁—S—, R^{2a}—NH—C(O)—Y₁—S—, NH₂—C(O)—O—Y₁—S—, R^{2a}—NH—C(O)—O—Y —S—, NH₂—C(S)—S—, NH₂—C(O)—S—, R^{2a}—NH—C(S)—S—, R^{2a}—NH—C(O)—S—, R^{2a}—S—C(S)—NH—, R^{2a}—S—C(O)—NH—, and

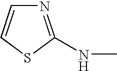

is optionally replaced by —S(O)— or —S(O)₂—; and

wherein the carbonyl moiety of said unsubstituted or substituted NH₂—C(O)—NH—Y₁—, NH₂—C(O)—NH—Y₁—O—, NH₂—C(O)—NH—Y₁—S—, R^{2a}—C(O)—NH—Y₁—, R^{2a}—C(O)—NH—Y₁—O—, R^{2a}—C(O)—NH—Y₁—S—, H—C(O)—NH—, H—C(O)—NH—Y₁—, H—C(O)—NH—Y₁—O—, H—C(O)—NH—Y₁—S—, R^{2a}—O—C(O)—NH—, R^{2a}—O—C(O)—NH—Y₁—, R^{2a}—O—C(O)—NH—Y₁—O—, R^{2a}—O—C(O)—NH—, R^{2a}—NH—Y₁—, (R^{2a})₂—N—(O)—NH—Y₁—, R^{2a}—NH—C(O)—NH—Y₁—O—, (R^{2a})₂—N—C(O)—NH—Y₁—O—, R^{2a}—NH—C(O)—NH—Y₁—S—, (R^{2a})₂—N—C(O)—NH—Y₁—S—, NH₂—C(O)—, NH₂—C(O)—Y₁—, NH₂—C(O)—Y₁—O—, NH₂—C(O)—Y₁—S—, R^{2a}—NH—C(O)—, R^{2a}—NH—C(O)—Y₁—, R^{2a}—NH—C(O)—Y—O—, R^{2a}—NH—C(O)—Y₁—S—, NH₂—C(O)—O—O—, NH₂—C(O)—O—Y₁—, NH₂—C(O)—O—Y₁—O—, NH₂—C(O)—O—Y₁—S—, R^{2a}—NH—C(O)—O—, R^{2a}—NH—C(O)—O—Y₁—, R^{2a}—NH—C(O)—O—Y₁—O—, R^{2a}—NH—C(O)—O—Y₁—S—, R^{2a}—NH—C(O)—NH—, R^{2a}—C(O)—NH—, NH₂—C(O)—S—, R^{2a}—NH—C(O)—S—, R^{2a}—S—C(O)—NH—, and R^{2a}—C(O)—

is optionally replaced by a thiocarbonyl moiety; provided that when R₂ is attached to

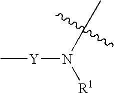

through a heteroatom, Y can not be a covalent bond.

In another particular embodiment of this invention, R² is —OC(O)(NHR^{2a}), —NHC(O)OR^{2a}, —C(O)R^{2a}, —C(O)(NHR^{2a}), or —NHC(O)H. In a more particular embodiment, R² is —OC(O)(NHR^{2a}), —NHC(O)OR^{2a}, —C(O)R^{2a}, —C(O)(NHR^{2a}) and R^{2a} is methyl or ethyl.

In another particular embodiment of this invention, R² is —NHC(O)OR^{2a}. In a more particular embodiment, R² is —NHC(O)OR^{2a} and R^{2a} is methyl or ethyl. In a most particular embodiment, R² is —NHC(O)OCH₃

In one embodiment of this invention, R^{2a} is straight or branched (C₁-C₁₂)alkyl, straight or branched (C₁-C₁₂)haloalkyl, (C₃-C₇)cycloalkyl, or straight or branched C₁-C₁₂ alkoxyalkyl, R^{2d} is (C₂-C₁₂)alkenyl, and R^{2e} is H, (C₁-C₆)alkyl, phenyl, heteroaryl, cyano, nitro, —S(O)R^{2a}, —S(O)₂R^{2a}, —S(O)₂NHR^{2a}, —S(O)₂NR^{2a}R^{2a}, —C(O)R^{2a}, —C(S)R^{2a}, —C(O)OR^{2a}, —C(S)OR^{2a}, —C(O)(NH₂), —C(O)(NHR^{2a}). In a particular embodiment, R^{2a} is methyl or ethyl.

Y₁ is a straight or branched C₁-C₁₂ alkylene optionally substituted with one or more halogens and optionally interrupted at an internal carbon atom with oxygen.

In one embodiment, A is a saturated or unsaturated 4-, 5-, 6-, or 7-membered ring which is optionally bridged by (CH₂)ₚ via bonds to two members of said ring, wherein said ring is composed of carbon atoms and 0-2 hetero atoms selected from the group consisting of 0, 1, or 2 nitrogen atoms, 0 or 1 oxygen atoms, and 0 or 1 sulfur atoms, said ring being optionally and independently substituted with zero to four halogen atoms, (C₁-C₆)alkyl groups, halo(C₁-C₆)alkyl groups or oxo groups such that when there is substitution with one oxo group on a carbon atom it forms a carbonyl group, and when there is substitution of one or two oxo groups on sulfur it forms sulfoxide or sulfone groups, respectively and p is 1 to 3.

In a particular embodiment, A is selected from phenyl, cyclohexyl, 1,3-dimethylpiperidine, and 2,4-dimethylmorpholine. In a more particular embodiment of, A is phenyl or 1,3-dimethylpiperidine.

In one embodiment of this invention, Y is a covalent bond or C₁-C₁₀ alkylene, C₁-C₁₀ alkenylene or C₁-C₁₀ alkynylene, each optionally substituted at one or more substitutable carbon atoms with halogen, cyano, hydroxy, (C₁-C₃)alkyl, (C₁-C₃)alkoxy or halo(C₁-C₃)alkoxy. In a particular embodiment of this invention, Y is a covalent bond. In another particular embodiment, Y is C₁-C₅ alkylene optionally substituted as described above. In another more particular embodiment, Y is a C₂-C₃ alkylene optionally substituted as described above.

In one embodiment of the invention, Q1, Q2, Q3, Q4, Q5, Q6:

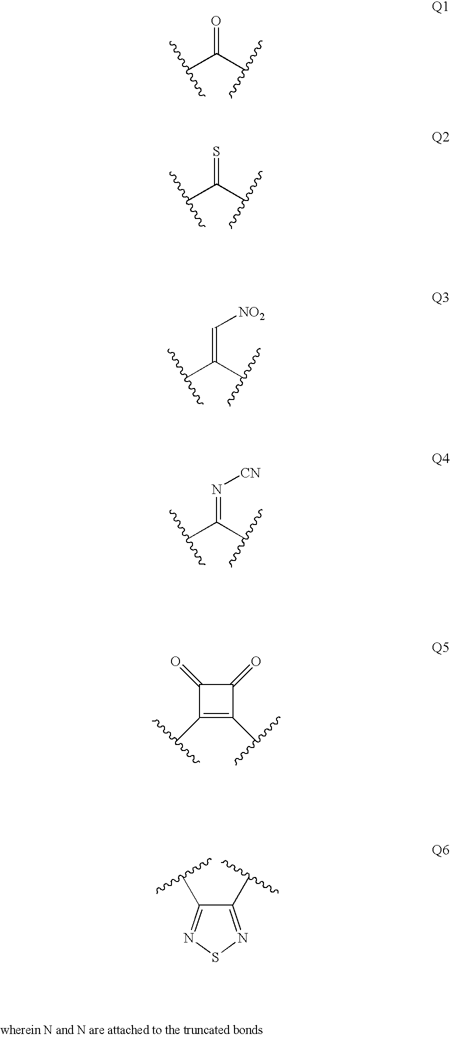

In a particular embodiment of the invention, Q is Q1: —C(O)—.

In one embodiment, R⁴ is H, (C₁-C₆)alkyl, halo(C₁-C₆)alkyl, (C₁-C₃)alkoxy(C₁-C₃)alkyl, or cyano(C₁-C₆)alkyl. In a particular embodiment of the invention, R⁴ is H.

In one embodiment, G is OH, OR^{e}, NH₂, NHR^{e}, NR^{e}R^{f}, C(═NH)NH₂, C(═NH)NHR^{e}, NHC(═NH)NH₂, or NHC(═NH)NHR^{e} and R^{e} and R^{f} are as described below.

In another particular embodiment of this invention, G is OH, NH₂ or NHR^{e}.

An a more particular embodiment, G is OH, NH₂ or NHR^{e} and R^{e} is a) (C₁-C₆)alkyl, halo(C₁-C₆)alkyl, (C₄-C₁₀)cycloalkylalkyl, (C₁-C₅)alkoxy(C₁-C₅)alkyl, or aminocarbonyl(C₁-C₆)alkyl or b) phenyl(C₁-C₂)alkyl optionally substituted with 1 to 3 groups independently selected from: fluorine, chlorine, cyano, (C₁-C₃)alkyl, halo(C₁-C₃)alkyl, (C₁-C₃)alkoxy, and halo(C₁-C₃)alkoxy; or c) R⁵ and R^{e} together are —CH₂—, —(CH₂)₂—, —(CH₂)₃—, or —(CH₂)₄—, optionally substituted with 1 or 2 groups independently selected from fluorine, (C₁-C₈)alkyl, halo(C₁-C₈)alkyl, (C₃-C₆)cycloalkyl, halo(C₃-C₆)cycloalkyl, hydroxy(C₃-C₆)cycloalkyl, (C₃-C₆)cycloalkyl(C₁-C₂)alkyl, halo(C₃-C₆)cycloalkyl(C₁-C₂)alkyl, hydroxy(C₃-C₆)cycloalkyl(C₁-C₂)alkyl, (C₁-C₈)alkoxy, halo(C₁-C₈)alkoxy, (C₃-C₆)cycloalkoxy, halo(C₃-C₆)cycloalkoxy, and heterocyclyl.

In another particular embodiment, G is NH₂ or NHR^{e}.

In another particular embodiment, G is NHR^{e} and R^{e}. In a more particular embodiment, G is NHR^{e} and R^{e} is methyl or R^{s} and R^{e} together are —(CH₂)₃-optionally substituted with C₁-C₄ alkyl or cyclohexyl. In a more particular embodiment, G is NHR^{e} and R^{e} is methyl.

In one embodiment of this invention, L is 1) a linear (C₂-C₄)alkyl chain when G is OH, OR^{e}, NH₂, NHR^{e}, NR^{e}R^{f}, NHC(═NH)NH₂, or NHC(═NH)NHR^{e}, or 2) a linear (C₁-C₃)alkyl chain when G is C(═NH)NH₂ or C(═NH)NHR^{e} and L is optionally substituted by 1-4 groups independently selected from R⁵, R^{5a}, R⁶, and R^{6a}; one or more of the carbon atoms of L may be part of a 3-, 4-, 5-, 6-, or 7-membered saturated ring composed of carbon atoms, and 0-2 hetero atoms selected from 0 or 1 nitrogen atoms, 0 or 1 oxygen atoms, and 0 or 1 sulfur atoms; said saturated ring being optionally substituted with up to four groups selected from halogen, (C₁-C₆)alkyl, halo(C₁-C₆)alkyl, (C₃-C₆)cycloalkyl, halo(C₃-C₆)cycloalkyl, (C₄-C₇)cycloalkylalkyl, halo(C₄-C₇)cycloalkylalkyl, and oxo, such that when there is substitution with one oxo group on a carbon atom it forms a carbonyl group and when there is substitution of one or two oxo groups on sulfur it forms sulfoxide or sulfone groups, respectively.

In another embodiment of this invention L is a C₂ alkyl chain, optionally substituted with R⁵ and R⁶.

In one embodiment, each of R⁵, R^{5a}, R⁶, and R^{6a} is independently 1) H, (C₁-C₁₂)alkyl, halo(C₁-C₁₂)alkyl, hydroxy(C₁-C₁₂)alkyl, (C₃-C₁₀)cycloalkyl, (C₃-C₁₀)cycloalkyl, (C₃-C₁₀)cycloalkylalkyl, halo(C₃-C₁₀)cycloalkylalkyl, hydroxy(C₃-C₁₀)cycloalkylalkyl, (C₁-C₂)alkyl(C₃-C₁₀)cycloalkylalkyl, halo(C₁-C₂)alkyl(C₃-C₁₀)cycloalkylalkyl, di(C₁-C₂)alkyl(C₃-C₁₀)cycloalkylalkyl, hydroxy(C₁-C₂)alkyl (C₃-C₁₀)cycloalkylalkyl, hydroxy di(C₁-C₂)alkyl(C₃-C₁₀)cycloalkylalkyl, (C₂-C₁₂)alkenyl, (C₅-C₈)cycloalkyl(C₁-C₃)alkenyl, (C₂-C₁₂)alkynyl, (C₃-C₈)cycloalkyl(C₁-C₃)alkynyl, (C₄-C₁₂)bicycloalkyl(C₁-C₃)alkyl, (C₈-C₁₄)tricycloalkyl(C₁-C₃)alkyl, (C₁-C₆)alkoxy(C₁-C₆)alkyl, halo(C₁-C₆)alkoxy(C₁-C₆)alkyl, (C₃-C₈)cycloalkoxy(C₁-C₃)alkyl, (C₁-C₆)alkylthio(C₁-C₆)alkyl, halo(C₁-C₆)alkylthio(C₁-C₆)alkyl, (C₃-C₈)cycloalkylthio(C₁-C₃)alkyl, saturated heterocyclyl, and saturated heterocyclyl(C₁-C₃)alkyl wherein (a) hydrogen atoms in these groups are optionally substituted by 1 to 6 groups independently selected from halogen, cyano, nitro, hydroxyl, (C₁-C₆)alkyl, (C₁-C₆)alkoxy, (C₃-C₆)cycloalkyl, (C₃-C₇)cycloalkylalkyl, halo(C₃-C₇)cycloalkylalkyl, (C₂-C₆)alkenyl, halo(C₂-C₆)alkenyl, (C₃-C₇)cycloalkylalkenyl, (C₂-C₆)alkynyl, halo(C₂-C₆)alkynyl, (C₃-C₇)cycloalkylalkoxy, halo(C₃-C₇)cycloalkylalkoxy, (C₃-C₇)cycloalkoxy, halo(C₁-C₆)alkyl, (C₃-C₇)cycloalkylalkynyl, halo(C₃-C₇)cycloalkylalkynyl, halo(C₁-C₆)alkoxy, halo(C₃-C₇)cycloalkyl, halo(C₃-C₇)cycloalkoxy, (C₁-C₆)alkylsulfonyl, aminocarbonyl and wherein (b) divalent sulfur atoms are optionally oxidized to sulfoxide or sulfone; or 2) phenyl, naphthyl, heteroaryl, phenyl(C₁-C₃)alkyl, phenoxymethyl, naphthyl(C₁-C₃)alkyl, and heteroaryl(C₁-C₃)alkyl, each optionally substituted with 1 to 3 groups independently selected from: fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkenyl, halo(C₂-C₆)alkenyl, (C₃-C₆)cycloalkylalkenyl, (C₂-C₆)alkynyl, halo(C₂-C₆)alkynyl, (C₃-C₆)cycloalkyl-(C₂-C₄)alkynyl, halo(C₃-C₇)cycloalkylalkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylhio, (C₄-C₇)cycloalkylalkylthio, halo(C₁-C₆)alkylthio, halo(C₃-C₆)cycloalkylhio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)-cycloalkylalkanesulfonyl, (C₁-C₆)alkylamino, di(C₁-C₆)alkylamino, (C₁-C₆)-alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)-cycloalkylalkoxy(C₁-C₆)alkyl, (C₁-C₈)alkylthio(C₁-C₆)alkyl, (C₃-C₈)cycloalkylhio(C₁-C₆)alkyl, (C₄-C₈)cycloalkylalkylthio(C₁-C₆)alkyl, halo(C₁-C₈)alkylthio(C₁-C₆)alkyl, halo(C₃-C₈)cycloalkylhio(C₁-C₆)alkyl, halo(C₄-C₈)-cycloalkylalkylthio(C₁-C₆)alkyl, (C₁-C₈)alkanesulfinyl(C₁-C₆)alkyl, (C₃-C₈)-cycloalkanesulfinyl(C₁-C₆)alkyl, (C₄-C₈)cycloalkyl-alkanesulfinyl(C₁-C₆)alkyl, halo(C₁-C₈)alkanesulfinyl(C₁-C₆)alkyl, halo(C₃-C₈)cycloalkanesulfinyl(C₁-C₆)alkyl, halo(C₄-C₈)cycloalkylalkanesulfinyl(C₁-C₆)alkyl, (C₁-C₈)alkane-sulfonyl(C₁-C₆)alkyl, (C₃-C₈)cycloalkanesulfonyl(C₁-C₆)alkyl, (C₄-C₈)cycloalkylalkanesulfonyl(C₁-C₆)alkyl, halo(C₁-C₆)alkanesulfonyl(C₁-C₆)alkyl, halo(C₃-C₈)cycloalkanesulfonyl(C₁-C₆)alkyl, halo(C₄-C₈)cycloalkylalkane-sulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylamino-carbonyl(C₁-C₆)alkyl, di(C₁-C₈)alkylaminocarbonyl(C₁-C₆)alkyl and (C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylamino-carboxy(C₁-C₆)alkyl and di(C₁-C₈)alkylaminocarboxy(C₁-C₆)alkyl, phenyl, naphthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, naphthyl(C₁-C₃)alkyl, heteroaryl(C₁-C₃)alkyl, and bicyclic heteroaryl(C₁-C₃)alkyl, wherein the aromatic and heteroaromatic groups are optionally substituted with 1 to 3 groups independently selected from fluorine, chlorine, cyano, (C₁-C₃)alkyl, halo(C₁-C₃)alkyl, (C₁-C₃)alkoxy, halo(C₁-C₃)-alkoxy, (C₁-C₃)alkanesulfonyl, and (C₁-C₃)alkoxycarbonyl.

In another particular embodiment, one of R⁵ and R⁶ is —H or methyl and the other is selected from a) H, (C₁-C₁₀)alkyl, (C₄-C₁₀)cycloalkylalkyl, halo(C₁-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁₀)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₅)alkyl, or saturated heterocyclyl(C₁-C₃)alkyl; or b) phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy.

In a more particular embodiment, R⁶ is —H or methyl and R⁵ is selected from a) H, (C₁-C₁₀)alkyl, (C₄-C₁₀)cycloalkylalkyl, halo(C₁-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁₀)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₅)alkyl, or saturated heterocyclyl(C₁-C₃)alkyl; or b) phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy.

In another more particular embodiment, R⁵ is —H or methyl and R⁶ is selected from a) H, (C₁-C₁₀)alkyl, (C₄-C₁₀)cycloalkylalkyl, halo(C₁-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁₀)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₅)alkyl, or saturated heterocyclyl(C₁-C₃)alkyl; or b) phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy.

In another particular embodiment, R⁶ is —H or methyl and R⁵ is selected from a) H, (C₁-C₁₀)alkyl, (C₄-C₁₀)cycloalkylalkyl, halo(C₁-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁₀)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₅)alkyl, or saturated heterocyclyl(C₁-C₃)alkyl; or b) phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy.

In another particular embodiment, R⁵ is —H or methyl and R⁶ is selected from a) H, (C₁-C₁₀)alkyl, (C₄-C₁₀)cycloalkylalkyl, halo(C₁-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁₀)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₅)alkyl, or saturated heterocyclyl(C₁-C₃)alkyl; or b) phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy.

In another particular embodiment, R⁵ is (C₁-C₇)alkyl, halo(C₁-C₇)alkyl, hydroxy(C₁-C₇)alkyl, cyclohexylmethyl, halocyclohexylmethyl, hydroxy cyclohexylmethyl, 2-(cyclohexyl)ethyl, (C₁-C₂)alkyl cyclohexylmethyl, di(C₁-C₂)alkyl cyclohexylmethyl, hydroxy(C₁-C₂)alkyl cyclohexylmethyl, hydroxy di(C₁-C₂)alkylcyclohexylmethyl, (3-noradamantyl)methyl, (tetrahydropyranyl)methyl, or oxepanyl methyl and R⁶ is —H or methyl.

In another particular embodiment, R⁶ is (C₁-C₇)alkyl, halo(C₁-C₇)alkyl, hydroxy(C₁-C₇)alkyl, cyclohexylmethyl, halocyclohexylmethyl, hydroxy cyclohexylmethyl, 2-(cyclohexyl)ethyl, (C₁-C₂)alkyl cyclohexylmethyl, di(C₁-C₂)alkyl cyclohexylmethyl, hydroxy(C₁-C₂)alkyl cyclohexylmethyl, hydroxy di(C₁-C₂)alkylcyclohexylmethyl, (3-noradamantyl)methyl, (tetrahydropyranyl)methyl, or oxepanyl methyl and R⁵ is —H or methyl.

In a more particular embodiment of this invention, R⁵ is cyclohexylmethyl, (tetrahydropyranyl)methyl, or oxepanyl methyl and R⁶ is —H. In another more particular embodiment of this invention, R⁶ is cyclohexylmethyl, (tetrahydropyranyl)methyl, or oxepanyl methyl and R⁵ is —H.

In one embodiment of this invention, R^{e} is a) (C₁-C₁₂)alkyl, (C₄-C₁₂)cycloalkylalkyl, halo(C₁-C₁₂)alkyl, halo(C₄-C₁₂)cycloalkylalkyl, (C₂-C₁₂)alkenyl, (C₅-C₁₂)cycloalkylalkenyl, halo(C₂-C₁₂)alkenyl, halo(C₅-C₁₂)cycloalkylalkenyl, (C₂-C₁₂)alkynyl, (C₅-C₁₂)cycloalkylalkynyl, halo(C₂-C₁₂)alkynyl, halo(C₅-C₁₂)cycloalkylalkynyl, (C₁-C₆)alkoxy(C₁-C₆)alkyl, halo(C₁-C₆)alkoxy(C₁-C₆)alkyl, (C₁-C₆)alkylthio(C₁-C₆)alkyl, halo(C₁-C₆)alkylthio(C₁-C₆)alkyl, (C₁-C₆)alkanesulfinyl(C₁-C₆)alkyl, halo(C₁-C₆)alkane-sulfinyl(C₁-C₆)alkyl, (C₁-C₆)alkanesulfonyl(C₁-C₆)alkyl, halo(C₁-C₆)alkanesulfonyl(C₁-C₆)alkyl, aminocarbonyl(C₁-C₆)alkyl, (C₁-C₆)alkylaminocarbonyl(C₁-C₆)alkyl, di(C₁-C₆)alkylamino-carbonyl(C₁-C₆)alkyl, cyano(C₁-C₆)alkyl, carboxy(C₁-C₆)alkyl, (C₁-C₆)alkoxycarbonyl(C₁-C₆)alkyl, saturated heterocyclyl, or saturated heterocyclyl(C₁-C₆)alkyl or b) phenyl, naphthyl, heteroaryl, phenyl(C₁-C₃)alkyl, naphthyl(C₁-C₃)alkyl, or heteroaryl(C₁-C₃)alkyl, each optionally substituted by 1 to 3 groups independently selected from: 1) fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)cycloalkyl-(C₂-C₄)alkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylhio, (C₄-C₇)cycloalkylalkylthio, halo(C₁-C₆)alkylthio, halo(C₃-C₆)cycloalkylhio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃-C₆)cycloalkane-sulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)-cycloalkylalkanesulfonyl, (C₁-C₆)alkylamino, di(C₁-C₆)alkylamino, (C₁-C₆)alkoxy-(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl and di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)-cycloalkylalkoxy(C₁-C₆)alkyl, (C₁-C₈)alkylthio(C₁-C₆)alkyl, (C₃-C₈)cycloalkylhio(C₁-C₆)alkyl, (C₄-C₈)cycloalkylalkylthio(C₁-C₆)alkyl, halo(C₁-C₈)alkylthio(C₁-C₆)alkyl, halo(C₃-C₈)cycloalkylhio(C₁-C₆)alkyl, halo(C₄-C₈)-cycloalkylalkylthio(C₁-C₆)alkyl, (C₁-C₈)alkanesulfinyl(C₁-C₆)alkyl, (C₃-C₈)-cycloalkanesulfinyl(C₁-C₆)alkyl, (C₄-C₈)cycloalkyl-alkanesulfinyl(C₁-C₆)alkyl, halo(C₁-C₈)alkanesulfinyl(C₁-C₆)alkyl, halo(C₃-C₈)cycloalkanesulfinyl(C₁-C₆)alkyl, halo(C₄-C₈)cycloalkylalkanesulfinyl(C₁-C₆)alkyl, (C₁-C₈)alkane-sulfonyl(C₁-C₆)alkyl, (C₃-C₈)cycloalkanesulfonyl(C₁-C₆)alkyl, (C₄-C₈) cycloalkylalkanesulfonyl(C₁-C₆)alkyl, halo(C₁-C₈)alkanesulfonyl(C₁-C₆)alkyl, halo(C₃-C₈)cycloalkanesulfonyl(C₁-C₆)alkyl, halo(C₄-C₈)cycloalkylalkane-sulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylamino-carbonyl(C₁-C₆)alkyl, di(C₁-C₈)alkylaminocarbonyl(C₁-C₆)alkyl(C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylamino-carboxy(C₁-C₆)alkyl and di(C₁-C₈)alkylaminocarboxy(C₁-C₆)alkyl; or 2) phenyl, naphthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, naphthyl(C₁-C₃)alkyl, heteroaryl(C₁-C₃)alkyl, and bicyclic heteroaryl(C₁-C₃)alkyl, each optionally substituted with 1 to 3 groups independently selected from fluorine, chlorine, cyano, (C₁-C₃)alkyl, halo(C₁-C₃)alkyl, (C₁-C₃)alkoxy, halo(C₁-C₃)alkoxy, (C₁-C₃)alkanesulfonyl, and (C₁-C₃)-alkoxycarbonyl; or b) R^{e} is a saturated divalent radical composed of carbon atoms, and 0, 1 or 2 hetero atoms selected from 0 or 1 nitrogen atoms, 0 or 1 oxygen atoms, and 0 or 1 sulfur atoms that is attached to any core carbon atom on L to form a saturated 3-, 4-, 5-, 6-, or 7-membered L-G ring; said L-G ring being optionally substituted with 1 to 4 groups selected from halogen, fluorine, (C₁-C₈)alkyl, halo(C₁-C₈)alkyl, (C₃-C₈)cycloalkyl, halo(C₃-C₈)cycloalkyl, hydroxy(C₃-C₈)cycloalkyl, (C₃-C₈)cycloalkyl(C₁-C₃)alkyl, halo(C₃-C₈)cycloalkyl(C₁-C₃)alkyl, hydroxy(C₃-C₈)cycloalkyl(C₁-C₃)alkyl, (C₁-C₈)alkoxy, halo(C₁-C₈)alkoxy, (C₃-C₈)cycloalkoxy, halo(C₃-C₈)cycloalkoxy, hydroxy(C₃-C₈)cycloalkoxy, (C₁-C₈)alkoxy(C₁-C₃)alkyl, halo(C₁-C₈)alkoxy(C₁-C₃)alkyl, (C₃-C₈)cycloalkoxy(C₁-C₃)alkyl, halo(C₃-C₈)cycloalkoxy(C₁-C₃)alkyl, hydroxy(C₃-C₈)cycloalkoxy(C₁-C₃)alkyl, (C₃-C₈)cycloalkyl(C₁-C₃)alkoxy(C₁-C₃)alkyl, halo(C₃-C₈)cycloalkyl(C₁-C₃)alkoxy(C₁-C₃)alkyl, hydroxy(C₃-C₈)cycloalkyl(C₁-C₃)alkoxy(C₁-C₃)alkyl, (C₁-C₈)alkylthio, halo(C₁-C₈)alkylthio, (C₃-C₈)cycloalkylthio, halo(C₃-C₈)cycloalkylthio, hydroxy(C₃-C₈)cycloalkylthio, (C₃-C₈)cycloalkyl(C₁-C₃)alkylthio, halo(C₃-C₈)cycloalkyl(C₁-C₃)alkylthio, hydroxy(C₃-C₈)cycloalkyl(C₁-C₃)alkylthio, (C₁-C₈)alkylthio(C₁-C₃)alkyl, halo(C₁-C₈)alkylthio(C₁-C₃)alkyl, (C₃-C₈)cycloalkylthio(C₁-C₃)alkyl, halo(C₃-C₈)cycloalkylthio(C₁-C₃)alkyl, hydroxy(C₃-C₈)cycloalkylthio(C₁-C₃)alkyl, (C₃-C₈)cycloalkyl(C₁-C₃)alkylthio(C₁-C₃)alkyl, halo(C₃-C₈)cycloalkyl(C₁-C₃)alkylthio(C₁-C₃)alkyl, hydroxy(C₃-C₈)cycloalkyl(C₁-C₃)alkylthio(C₁-C₃)alkyl, heterocyclyl, and oxo.

In another particular embodiment of this invention, R^{e} is a) (C₁-C₆)alkyl, halo(C₁-C₆)alkyl, (C₄-C₁₀)cycloalkylalkyl, (C₁-C₅)alkoxy(C₁-C₅)alkyl, or aminocarbonyl(C₁-C₆)alkyl or b) phenyl(C₁-C₂)alkyl optionally substituted with 1 to 3 groups independently selected from: fluorine, chlorine, cyano, (C₁-C₃)alkyl, halo(C₁-C₃)alkyl, (C₁-C₃)alkoxy, and halo(C₁-C₃)alkoxy; or c) R⁵ and R^{e} together are —CH₂—, —(CH₂)₂—, —(CH₂)₃—, or —(CH₂)₄—, optionally substituted with 1 or 2 groups independently selected from fluorine, (C₁-C₈)alkyl, halo(C₁-C₈)alkyl, (C₃-C₆)cycloalkyl, halo(C₃-C₆)cycloalkyl, hydroxy(C₃-C₆)cycloalkyl, (C₃-C₆)cycloalkyl(C₁-C₂)alkyl, halo(C₃-C₆)cycloalkyl(C₁-C₂)alkyl, hydroxy(C₃-C₆)cycloalkyl(C₁-C₂)alkyl, (C₁-C₈)alkoxy, halo(C₁-C₈)alkoxy, (C₃-C₆)cycloalkoxy, halo(C₃-C₆)cycloalkoxy, and heterocyclyl.

In another particular embodiment, R^{e} is methyl or R⁵ and R^{e} together are —(CH₂)₃— optionally substituted with C₁-C₄ alkyl or cyclohexyl. In a more particular embodiment, R^{e} is methyl or R⁶ and R^{e} together are —(CH₂)₃— optionally substituted with C₁-C₄ alkyl or cyclohexyl. In a most particular embodiment, R^{e} is methyl.

In one embodiment, R^{f} is (C₁-C₆)alkyl or halo(C₁-C₆)alkyl.

In a first specific embodiment, the aspartic protease inhibitor of the invention is represented by Structural Formulas (Ia), (Ib) and (Ic) or an enantiomer, diastereomer or a pharmaceutically acceptable salt of the aspartic protease inhibitor represented by Structural Formula (Ia):

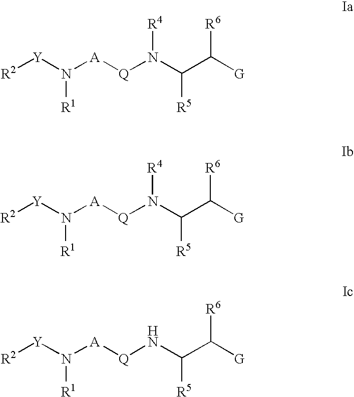

Values and particular values for the variables in Structural Formulas (Ia), (Ib) and (Ic) are as provided for Structural Formula (I) above.

A first set of values for Structural Formulas (Ia), (Ib) and (Ic) is provided in the following paragraphs:

R² is (C₁-C₁₂)alkyl, (C₂-C₁₂)alkenyl, (C₂-C₁₂)alkynyl, (C₃-C₇)cycloalkyl, R^{2a}—O—, R^{2d}—O—, R^{2a}—S—, R^{2a}—O—Y₁, R^{2a}—S—Y₁—, R^{2a}—O—Y₁—O—, R^{2a}—O—Y₁—S—, R^{2a}—S—Y₁—O—, R^{2a}—S—Y₁—S—, R^{2a}—O—Y₁—O—Y —, NH₂—C(O)—NH—Y₁—, NH₂—C(O)—NH—Y₁—O—, NH₂—C(O)—NH—Y₁—S—, R^{2a}—C(O)—NH—Y₁—, R^{2a}—C(O)—NH—Y₁—, R^{2a}—C(O)—NH—Y₁—S—, NH₂—S(O)₂—NH—Y₁—, NH₂—S(O)₂—NH—Y—O—, NH₂—S(O)₂—NH—Y —S—, R^{2a}—S(O)₂—NH—Y₁—, R^{2a}—S(O)₂—NH—Y—O—, R^{2a}—S(O)₂—NH—Y₁—S—, H—C(O)—NH—, H—C(O)—NH—Y₁—, H—C(O)—NH—Y₁—O—, H—C(O)—NH—Y₁—S—, R^{2a}—O—C(O)—NH—, R^{2a}—O—C(S)—NH—, R^{2a}—O—C(O)—NH—Y₁—, R^{2a}—C(O)—NH—Y₁—O—, R^{2a}—O—C(O)—NH—Y₁—S—, R^{2a}—NH—C(O)—NH—Y₁—, (R^{2a})₂—N—C(O)—NH—Y₁—, R^{2a}—NH—C(O)—NH—Y₁—O—, (R^{2a})₂—N—C(O)—NH—Y₁—O—, R^{2a}—NH—C(O)—NH—Y₁—S—, (R^{2a})₂—N—C(O)—NH—Y₁—S—, NH₂—C(O)—, NH₂—C(S)—, NH₂—C(O)—Y₁—, NH₂—C(O)—Y₁—O—, NH₂—C(O)—Y₁—S—, R^{2a}—NH—C(O)—, R^{2a}—NH—C(S)—, R^{2a}—NH—C(O)—Y₁—, R^{2a}—NH—C(O)—Y₁—O—, R^{2a}—NH—C(O)—Y₁—S—, NH₂—C(O)—O—, NH₂—C(S)—O—, NH₂—C(O)—O—Y₁—, NH₂—C(O)—O—Y₁—O—, NH₂—C(O)—O—Y₁—S—, R^{2a}—NH—C(O)—O—, R^{2a}—NH—C(S)—O—, R^{2a}—NH—C(O)—O—Y₁—, R^{2a}—NH—C(O)—O—Y₁—, R^{2a}—NH—C(O)—O—Y₁—S—, R^{2a}—NH—C(O)—NH—, R^{2a}—C(O)—NH—, NH₂—C(S)—S—, NH₂—C(O)—S—, R^{2a}—NH—C(S)—S—, R^{2a}—NH—C(O)—S—, R^{2a}—S—C(S)—NH—, R^{2a}—S—C(O)—NH—, R^{2a}—C(O)—, R^{2a}—C(S)—, NHC(═NR^{2e})(NH₂), —NHC(═NR^{2e})(NHR^{2a})

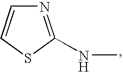

and

R^{2a} is straight or branched (C₁-C₁₂)alkyl, straight or branched (C₁-C₁₂)haloalkyl, (C₃-C₇)cycloalkyl, or straight or branched C₁-C₁₂ alkoxyalkyl, R^{2d}═(C₂-C₁₂)alkenyl, and R^{2e} is H, (C₁-C₆)alkyl, phenyl, heteroaryl, cyano, nitro, —S(O)R^{2a}, —S(O)₂R^{2a}, —S(O)₂NHR^{2a}, —S(O)₂NR^{2a}R^{2a}, —C(O)R^{2a}, —C(S)R^{2a}, —C(O)OR^{2a}, —C(S)OR^{2a}, —C(O)(NH₂), —C(O)(NHR^{2a}), Y₁ is a straight or branched C₁-C₁₂ alkylene optionally substituted with one or more halogens and optionally interrupted at an internal carbon atom with oxygen;

R² can be substituted by at least one of:
a) 1 to 6 halogen atoms; or
b) one substituent selected from the group consisting of cyano, hydroxyl, (C₁-C₃)alkoxy, (C₃-C₆)cycloalkyl, (C₃-C₆)cycloalkoxy, halo(C₁-C₃)alkoxy, halo(C₃-C₆)cycloalkyl and halo(C₃-C₆)cycloalkoxy; and

wherein the thio-moiety of said unsubstituted or substituted R^{2a}—S—, R^{2a}—S—Y₁—, R^{2a}—O—Y₁—S—, R^{2a}—S—Y₁—O—, R^{2a}—S—Y₁—S—, NH₂—C(O)—NH—Y₁—S—, R^{2a}—C(O)—NH—Y₁—S—, NH₂—S(O)₂—NH—Y₁—S—, R^{2a}—S(O)₂—NH—Y₁—S—, H—C(O)—NH—Y₁—S—, R^{2a}—O—C(O)—NH—Y₁—S—, R^{2a}—NH—C(O)—NH—Y₁—S—, (R^{2a})₂—N—C(O)—NH—Y₁—S—, NH₂—C(O)—Y₁—S—, R^{2a}—NH—C(O)—Y₁—S—, NH₂—C(O)—O—Y₁—S—, R^{2a}—NH—C(O)—O—Y₁—S—, NH₂—C(S)—S—, NH₂—C(O)—S—, R^{2a}—NH—C(S)—S—, R^{2a}—NH—C(O)—S—, R^{2a}—S—C(S)—NH—, R^{2a}—S—C(O)—NH—, and

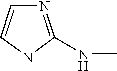

is optionally replaced by —S(O)— or —S(O)₂—; and

wherein the carbonyl moiety of said unsubstituted or substituted NH₂—C(O)—NH—Y₁—, NH₂—C(O)—NH—Y₁—O—, NH₂—C(O)—NH—Y₁—S—, R^{2a}—C(O)—NH—Y₁, R^{2a}—C(O)—NH—Y₁—O—, R^{2a}—C(O)—NH—Y₁—S—, H—C(O)—NH—, H—C(O)—NH—Y₁—, H—C(O)—NH—Y₁—O—, H—C(O)—NH—Y₁—S—, R^{2a}—O—C(O)—NH—, R^{2a}—O—C(O)—NH—Y₁—, R^{2a}—O—C(O)—NH—Y₁—, R^{2a}—O—C(O)—NH—Y₁—S—, R^{2a}—NH—C(O)—NH—Y₁—, (R^{2a})₂—N—C(O)—NH—Y₁—, R^{2a}—NH—C(O)—NH—Y₁—O—, (R^{2a})₂—N—C(O)—NH—Y₁—O—, R^{2a}—NH—C(O)—NH—Y₁—S—, (R^{2a})₂—N—C(O)—NH—Y₁—S—, NH₂—C(O)—, NH₂—C(O)—Y₁—, NH₂—C(O)—Y₁—O—, NH₂—C(O)—Y₁—S—, R^{2a}—NH—C(O)—, R^{2a}—NH—C(O)—Y₁—, R^{2a}—NH—C(O)—Y₁—O—, R^{2a}—NH—C(O)—Y₁—S—, NH₂—C(O)—O—, NH₂—C(O)—O—Y₁—, NH₂—C(O)—O—Y₁—, NH₂—C(O)—O—Y₁—S—, R^{2a}—NH—C(O)—O—, R^{2a}—NH—C(O)—O—Y₁—, —R^{2a}—NH—C(O)—O—Y₁—O—, R^{2a}—NH—C(O)—O—Y₁—S—, R^{2a}—NH—C(O)—NH—, R^{2a}—C(O)—NH—, NH₂—C(O)—S—, R^{2a}—NH—C(O)S—, R^{2a}—S—C(O)—NH—, and R^{2a}—C(O)—

is optionally replaced by a thiocarbonyl moiety;

provided that when R₂ is attached to

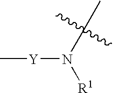

through a heteroatom, Y can not be a covalent bond; and

the remainder of the values and particular values for Structural Formulas (Ia), (Ib) and (Ic) are as described for Structural Formula I.

A second set of values for Structural Formulas (Ia), (Ib) and (Ic) is provided in the following paragraphs:

R² is —OC(O)(NHR^{2a}), —NHC(O)OR^{2a}, —C(O)R^{2a}, —C(O)(NHR^{2a}) or —NHC(O)H; and

the remainder of the values and particular values for Structural Formulas (Ia), (Ib) and (Ic) are as described for Structural Formula I.

A third set of values for Structural Formulas (Ia), (Ib) and (Ic) is provided in the following paragraphs:

R² is —OC(O)(NHR^{2a}), —NHC(O)OR^{2a}, —C(O)R^{2a}, —C(O)(NHR^{2a}), or —NHC(O)HR^{2a}

R^{2a} is methyl or ethyl; and

the values and particular values for Structural Formulas (Ia), (Ib) and (Ic) are as described for Structural Formula (I).

A fourth set of values for Structural Formulas (Ia), (Ib) and (Ic) is provided in the following paragraphs:

R² is —NHC(O)OR^{2a};

R^{2a} is methyl or ethyl; and

the remainder of the values and particular values for Structural Formulas (Ia), (Ib) and (Ic) are as described for Structural Formula (I).

A fifth set of values for Structural Formulas (Ia), (Ib) and (Ic) is provided in the following paragraphs:

R² is —NHC(O)OCH₃; and

the remainder of the values and particular values for Structural Formulas (Ia), (Ib) and (Ic) are as described for Structural Formula (I).

A sixth set of values for Structural Formulas (Ia), (Ib) and (Ic) is provided in the following paragraphs:

G is OH, NH₂ or NHR^{e};

R^{e} is a) (C₁-C₆)alkyl, halo(C₁-C₆)alkyl, (C₄-C₁₀)cycloalkylalkyl, (C₁-C₅)alkoxy(C₁-C₅)alkyl, or aminocarbonyl(C₁-C₆)alkyl or b) phenyl(C₁-C₂)alkyl optionally substituted with 1 to 3 groups independently selected from: fluorine, chlorine, cyano, (C₁-C₃)alkyl, halo(C₁-C₃)alkyl, (C₁-C₃)alkoxy, and halo(C₁-C₃)alkoxy; or c) R⁵ and R^{e} together are —CH₂—, —(CH₂)₂—, —(CH₂)₃—, or —(CH₂)₄—, optionally substituted with 1 or 2 groups independently selected from fluorine, (C₁-C₈)alkyl, halo(C₁-C₈)alkyl, (C₃-C₆)cycloalkyl, halo(C₃-C₆)cycloalkyl, hydroxy(C₃-C₆)cycloalkyl, (C₃-C₆)cycloalkyl(C₁-C₂)alkyl, halo(C₃-C₆)cycloalkyl(C₁-C₂)alkyl, hydroxy(C₃-C₆)cycloalkyl(C₁-C₂)alkyl, (C₁-C₈)alkoxy, halo(C₁-C₈)alkoxy, (C₃-C₆)cycloalkoxy, halo(C₃-C₆)cycloalkoxy, and heterocyclyl;

and the remainder of the values and particular values for Structural Formulas (Ia), (Ib) and (Ic) are as described for Structural Formula (I).

In a second specific embodiment, the aspartic protease inhibitor of the invention is represented by Structural Formula (II) or Structural Formula (IIa), or an enantiomer, diastereomer or a pharmaceutically acceptable salt of the aspartic protease inhibitor represented by Structural Formula (II) or (IIa):

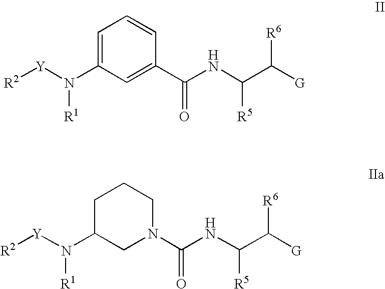

Values and particular values for the variables in Structural Formula (II) and Structural Formula (IIa) are as provided for Structural Formula (I) above.

A first set of values for Structural Formula (II) and Structural Formula (IIa) is provided in the following paragraphs:

one of R⁵ and R⁶ is —H or methyl and the other is as described for Structural Formula (I); and

the remainder of the values and particular values for Structural Formula (II) and (IIa) are as described for Structural Formula (I).

A second set of values for Structural Formula (II) and Structural Formula (IIa) is provided in the following paragraphs:

R⁶ is —H or methyl; and

the remainder of the values and particular values for Structural Formulas (II) and (IIa) are as described for Structural Formula (I).

A third set of values for Structural Formula (II) and Structural Formula (IIa) is provided in the following paragraphs:

R⁵ is —H or methyl; and

the remainder of the values and particular values for Structural Formulas (II) and (IIa) are as described for Structural Formula (I).

A fourth set of values for Structural Formula (II) and Structural Formula (IIa) is provided in the following paragraphs:

one of R⁵ and R⁶ is H or methyl and the other is selected from a) H, (C₁-C₁₀)alkyl, (C₄-C₁₀)cycloalkylalkyl, halo(C₁-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁₀)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₅)alkyl, or saturated heterocyclyl(C₁-C₃)alkyl; or b) phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy; and

the remainder of the values and particular values for Structural Formulas (II) and (IIa) are as described for Structural Formula (I).

A fifth set of values for Structural Formula (II) and Structural Formula (IIa) is provided in the following paragraphs:

R⁶ is H or methyl and R⁵ is selected from a) H, (C₁-C₁₀)alkyl, (C₄-C₁₀)cycloalkylalkyl, halo(C₁-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₅)alkyl, or saturated heterocyclyl(C₁-C₃)alkyl; or b) phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy; and

the remainder of the values and particular values for Structural Formulas (II) and (IIa) are as described for Structural Formula (I).

A sixth set of values for Structural Formula (II) and Structural Formula (IIa) is provided in the following paragraphs:

R⁵ is H or methyl and R⁶ is selected from a) H, (C₁-C₁₀)alkyl, (C₄-C₁₀)cycloalkylalkyl, halo(C₁-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁₀)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₅)alkyl, or saturated heterocyclyl(C₁-C₃)alkyl; or b) phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy; and

the remainder of the values and particular values for Structural Formulas (II) and (IIa) are as described for Structural Formula (I).

A seventh set of values for Structural Formula (II) and Structural Formula (IIa) is provided in the following paragraphs:

G is OH, NH₂ or NHR^{e};

R^{e} is a) (C₁-C₆)alkyl, halo(C₁-C₆)alkyl, (C₄-C₁₀)cycloalkylalkyl, (C₁-C₅)alkoxy(C₁-C₅)alkyl, or aminocarbonyl(C₁-C₆)alkyl or b) phenyl(C₁-C₂)alkyl optionally substituted with 1 to 3 groups independently selected from: fluorine, chlorine, cyano, (C₁-C₃)alkyl, halo(C₁-C₃)alkyl, (C₁-C₃)alkoxy, and halo(C₁-C₃)alkoxy; or c) R⁵ and R^{e} together are —CH₂—, —(CH₂)₂—, —(CH₂)₃—, or —(CH₂)₄—, optionally substituted with 1 or 2 groups independently selected from fluorine, (C₁-C₈)alkyl, halo(C₁-C₈)alkyl, (C₃-C₆)cycloalkyl, halo(C₃-C₆)cycloalkyl, hydroxy(C₃-C₆)cycloalkyl, (C₃-C₆)cycloalkyl(C₁-C₂)alkyl, halo(C₃-C₆)cycloalkyl(C₁-C₂)alkyl, hydroxy(C₃-C₆)cycloalkyl(C₁-C₂)alkyl, (C₁-C₈)alkoxy, halo(C₁-C₈)alkoxy, (C₃-C₆)cycloalkoxy, halo(C₃-C₆)cycloalkoxy, and heterocyclyl; and the remainder of the values and particular values for Structural Formulas (II) and (IIa) are as described for Structural Formula (I).

A eighth set of values for Structural Formula (II) and Structural Formula (IIa) is provided in the following paragraphs:

G is OH, NH₂ or NHR^{e};

R^{e} is a) (C₁-C₆)alkyl, halo(C₁-C₆)alkyl, (C₄-C₁₀)cycloalkylalkyl, (C₁-C₅)alkoxy(C₁-C₅)alkyl, or aminocarbonyl(C₁-C₆)alkyl or b) phenyl(C₁-C₂)alkyl optionally substituted with 1 to 3 groups independently selected from: fluorine, chlorine, cyano, (C₁-C₃)alkyl, halo(C₁-C₃)alkyl, (C₁-C₃)alkoxy, and halo(C₁-C₃)alkoxy; or c) R⁵ and R^{e} together are —CH₂—, —(CH₂)₂—, —(CH₂)₃—, or —(CH₂)₄—, optionally substituted with 1 or 2 groups independently selected from fluorine, (C₁-C₈)alkyl, halo(C₁-C₈)alkyl, (C₃-C₆)cycloalkyl, halo(C₃-C₆)cycloalkyl, hydroxy(C₃-C₆)cycloalkyl, (C₃-C₆)cycloalkyl(C₁-C₂)alkyl, halo(C₃-C₆)cycloalkyl(C₁-C₂)alkyl, hydroxy(C₃-C₆)cycloalkyl(C₁-C₂)alkyl, (C₁-C₈)alkoxy, halo(C₁-C₈)alkoxy, (C₃-C₆)cycloalkoxy, halo(C₃-C₆)cycloalkoxy, and heterocyclyl;

and the remainder of the values and particular values for Structural Formulas (II) and (IIa) are as described for the fourth set of values for Structural Formulas (II) and (IIa).

A ninth set of values for Structural Formula (II) and Structural Formula (IIa) is provided in the following paragraphs:

G is OH, NH₂ or NHR^{e};

R^{e} is a) (C₁-C₆)alkyl, halo(C₁-C₆)alkyl, (C₄-C₁₀)cycloalkylalkyl, (C₁-C₅)alkoxy(C₁-C₅)alkyl, or aminocarbonyl(C₁-C₆)alkyl or b) phenyl(C₁-C₂)alkyl optionally substituted with 1 to 3 groups independently selected from: fluorine, chlorine, cyano, (C₁-C₃)alkyl, halo(C₁-C₃)alkyl, (C₁-C₃)alkoxy, and halo(C₁-C₃)alkoxy; or c) R⁵ and R^{e} together are —CH₂—, —(CH₂)₂—, —(CH₂)₃—, or —(CH₂)₄—, optionally substituted with 1 or 2 groups independently selected from fluorine, (C₁-C₈)alkyl, halo(C₁-C₈)alkyl, (C₃-C₆)cycloalkyl, halo(C₃-C₆)cycloalkyl, hydroxy(C₃-C₆)cycloalkyl, (C₃-C₆)cycloalkyl(C₁-C₂)alkyl, halo(C₃-C₆)cycloalkyl(C₁-C₂)alkyl, hydroxy(C₃-C₆)cycloalkyl(C₁-C₂)alkyl, (C₁-C₈)alkoxy, halo(C₁-C₈)alkoxy, (C₃-C₆)cycloalkoxy, halo(C₃-C₆)cycloalkoxy, and heterocyclyl;

and the remainder of the values and particular values for Structural Formulas (II) and (IIa) are as described for the fifth set of values for Structural Formulas (II) and (IIa).

A tenth set of values for Structural Formula (II) and Structural Formula (IIa) is provided in the following paragraphs:

G is OH, NH₂ or NHR^{e};

R^{e} is a) (C₁-C₆)alkyl, halo(C₁-C₆)alkyl, (C₄-C₁₀)cycloalkylalkyl, (C₁-C₅)alkoxy(C₁-C₅)alkyl, or aminocarbonyl(C₁-C₆)alkyl or b) phenyl(C₁-C₂)alkyl optionally substituted with 1 to 3 groups independently selected from: fluorine, chlorine, cyano, (C₁-C₃)alkyl, halo(C₁-C₃)alkyl, (C₁-C₃)alkoxy, and halo(C₁-C₃)alkoxy; or c) R^{s} and R^{e} together are —CH₂—, —(CH₂)₂—, —(CH₂)₃—, or —(CH₂)₄—, optionally substituted with 1 or 2 groups independently selected from fluorine, (C₁-C₈)alkyl, halo(C₁-C₈)alkyl, (C₃-C₆)cycloalkyl, halo(C₃-C₆)cycloalkyl, hydroxy(C₃-C₆)cycloalkyl, (C₃-C₆)cycloalkyl(C₁-C₂)alkyl, halo(C₃-C₆)cycloalkyl(C₁-C₂)alkyl, hydroxy(C₃-C₆)cycloalkyl(C₁-C₂)alkyl, (C₁-C₈)alkoxy, halo(C₁-C₈)alkoxy, (C₃-C₆)cycloalkoxy, halo(C₃-C₆)cycloalkoxy, and heterocyclyl;

and the remainder of the values and particular values for Structural Formulas (II) and (IIa) are as described for the sixth set of values for Structural Formulas (II) and (IIa).

In a third specific embodiment, the aspartic protease inhibitor of the invention is represented by Structural Formulas (III)-(VII) and Structural Formulas (IIIa)-(VIIa), or enantiomers, diastereomers or a pharmaceutically acceptable salt thereof:

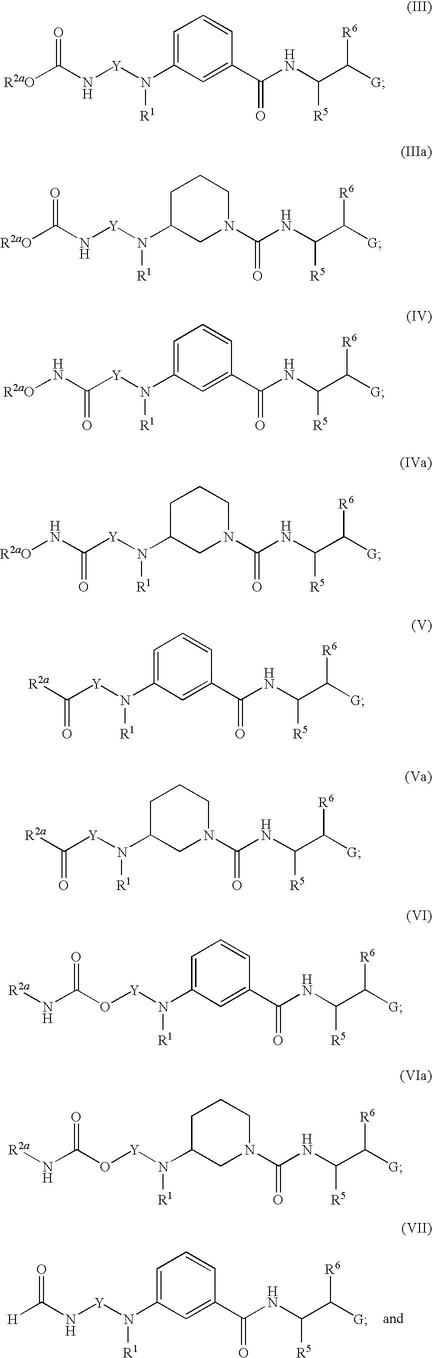

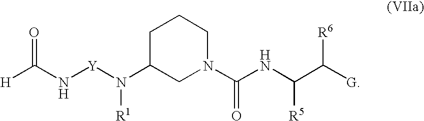

Values and particular values for the variables in Structural Formulas (III)-(VII) and Structural Formulas (IIIa)-(VIIa) are as provided for Structural Formula (I) above. A first set of values of Structural Formulas (III)-(VII) and Structural Formulas (IIIa)-(VIIa) is described in the following paragraphs:

one of R⁵ and R⁶ is —H or methyl and the other is as described for Structural Formula (I); and the remainder of the values and particular values for Structural Formulas (III)-(VII) and Structural Formulas (IIIa)-(VIIa) are as described for Structural Formula (I).

A second set of values and particular values for Structural Formulas (III)-(VII) and Structural Formulas (IIIa)-(VIIa) is described in the following paragraphs:

R⁶ is —H or methyl; and
the remainder of the values and particular values for Structural Formulas (III)-(VII) and Structural Formulas (IIIa)-(VIIa) are as described for Structural Formula (I).

A third set of values for Structural Formulas (III)-(VII) and Structural Formulas (IIIa)-(VIIa) is described in the following paragraphs:

R⁵ is —H or methyl; and

the remainder of the values and particular values for Structural Formulas (III)-(VII) and Structural Formulas (IIIa)-(VIIa) are as described for Structural Formula (I).

A fourth set of values for Structural Formulas (III)-(VII) and Structural Formulas (IIIa)-(VIIa) is described in the following paragraphs:

one of R⁵ and R⁶ is H or methyl and the other is a) H, (C₁-C₁₀)alkyl, (C₄-C₁₀)cycloalkylalkyl, halo(C₁-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁₀)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₅)alkyl, or saturated heterocyclyl(C₁-C₃)alkyl; or b) phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy; and

the remainder of the values and particular values for Structural Formulas (III)-(VII) and Structural Formulas (IIIa)-(VIIa) are as described for Structural Formula (I).

A fifth set of values for Structural Formulas (III)-(VII) and Structural Formulas (IIIa)-(VIIa) is described in the following paragraphs:

R⁶ is H or methyl and R⁵ is a) H, (C₁-C₁₀)alkyl, (C₄-C₁₀)cycloalkylalkyl, halo(C₁-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁₀)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₅)alkyl, or saturated heterocyclyl(C₁-C₃)alkyl; or b) phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy; and

the remainder of the values and particular values for Structural Formulas (III)-(VII) and Structural Formulas (IIIa)-(VIIa) are as described for Structural Formula (I).

A sixth set of values for Structural Formulas (III)-(VII) and Structural Formulas (IIIa)-(VIIa) is described in the following paragraphs:

R⁵ is H or methyl and R⁶ is a) H, (C₁-C₁₀)alkyl, (C₄-C₁₀)cycloalkylalkyl, halo(C₁-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁₀)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₅)alkyl, or saturated heterocyclyl(C₁-C₃)alkyl; or b) phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy; and

the remainder of the values and particular values for Structural Formulas (III)-(VII) and Structural Formulas (IIIa)-(VIIa) are as described for Structural Formula (I).

A seventh set of values for (III)-(VII) and Structural Formulas (IIIa)-(VIIa) is provided in the following paragraphs:

G is OH, NH₂ or NHR^{e};

R^{e} is a) (C₁-C₆)alkyl, halo(C₁-C₆)alkyl, (C₄-C₁₀)cycloalkylalkyl, (C₁-C₅)alkoxy(C₁-C₅)alkyl, or aminocarbonyl(C₁-C₆)alkyl or b) phenyl(C₁-C₂)alkyl optionally substituted with 1 to 3 groups independently selected from: fluorine, chlorine, cyano, (C₁-C₃)alkyl, halo(C₁-C₃)alkyl, (C₁-C₃)alkoxy, and halo(C₁-C₃)alkoxy; or c) R⁵ and R^{e} together are —CH₂—, —(CH₂)₂—, —(CH₂)₃—, or —(CH₂)₄—, optionally substituted with 1 or 2 groups independently selected from fluorine, (C₁-C₈)alkyl, halo(C₁-C₈)alkyl, (C₃-C₆)cycloalkyl, halo(C₃-C₆)cycloalkyl, hydroxy(C₃-C₆)cycloalkyl, (C₃-C₆)cycloalkyl(C₁-C₂)alkyl, halo(C₃-C₆)cycloalkyl(C₁-C₂)alkyl, hydroxy(C₃-C₆)cycloalkyl(C₁-C₂)alkyl, (C₁-C₈)alkoxy, halo(C₁-C₈)alkoxy, (C₃-C₆)cycloalkoxy, halo(C₃-C₆)cycloalkoxy, and heterocyclyl;

and the remainder of the values and particular values for (III)-(VII) and Structural Formulas (IIIa)-(VIIa) are as described for Structural Formula (I).

A eighth set of values for (III)-(VII) and Structural Formulas (IIIa)-(VIIa) is provided in the following paragraphs:

G is OH, NH₂ or NHR^{e};

R^{e} is a) (C₁-C₆)alkyl, halo(C₁-C₆)alkyl, (C₄-C₁₀)cycloalkylalkyl, (C₁-C₅)alkoxy(C₁-C₅)alkyl, or aminocarbonyl(C₁-C₆)alkyl or b) phenyl(C₁-C₂)alkyl optionally substituted with 1 to 3 groups independently selected from: fluorine, chlorine, cyano, (C₁-C₃)alkyl, halo(C₁-C₃)alkyl, (C₁-C₃)alkoxy, and halo(C₁-C₃)alkoxy; or c) R⁵ and R^{e} together are —CH₂—, —(CH₂)₂—, —(CH₂)₃—, or —(CH₂)₄—, optionally substituted with 1 or 2 groups independently selected from fluorine, (C₁-C₈)alkyl, halo(C₁-C₈)alkyl, (C₃-C₆)cycloalkyl, halo(C₃-C₆)cycloalkyl, hydroxy(C₃-C₆)cycloalkyl, (C₃-C₆)cycloalkyl(C₁-C₂)alkyl, halo(C₃-C₆)cycloalkyl(C₁-C₂)alkyl, hydroxy(C₃-C₆)cycloalkyl(C₁-C₂)alkyl, (C₁-C₈)alkoxy, halo(C₁-C₈)alkoxy, (C₃-C₆)cycloalkoxy, halo(C₃-C₆)cycloalkoxy, and heterocyclyl;

and the remainder of the values and particular values for Structural Formulas (III)-(VII) and Structural Formulas (IIIa)-(VIIa) are as described for the fourth set of values for Structural Formulas (III)-(VII) and Structural Formulas (IIIa)-(VIIa).

A ninth set of values for Structural Formulas (III)-(VII) and Structural Formulas (IIIa)-(VIIa) is provided in the following paragraphs:

G is OH, NH₂ or NHR^{e};

R^{e} is a) (C₁-C₆)alkyl, halo(C₁-C₆)alkyl, (C₄-C₁₀)cycloalkylalkyl, (C₁-C₈)alkoxy(C₁-C₅)alkyl, or aminocarbonyl(C₁-C₆)alkyl or b) phenyl(C₁-C₂)alkyl optionally substituted with 1 to 3 groups independently selected from: fluorine, chlorine, cyano, (C₁-C₃)alkyl, halo(C₁-C₃)alkyl, (C₁-C₃)alkoxy, and halo(C₁-C₃)alkoxy; or c) R⁵ and R^{e} together are —CH₂—, —(CH₂)₂—, —(CH₂)₃—, or —(CH₂)₄—, optionally substituted with 1 or 2 groups independently selected from fluorine, (C₁-C₈)alkyl, halo(C₁-C₈)alkyl, (C₃-C₆)cycloalkyl, halo(C₃-C₆)cycloalkyl, hydroxy(C₃-C₆)cycloalkyl, (C₃-C₆)cycloalkyl(C₁-C₂)alkyl, halo(C₃-C₆)cycloalkyl(C₁-C₂)alkyl, hydroxy(C₃-C₆)cycloalkyl(C₁-C₂)alkyl, (C₁-C₈)alkoxy, halo(C₁-C₈)alkoxy, (C₃-C₆)cycloalkoxy, halo(C₃-C₆)cycloalkoxy, and heterocyclyl;

and the remainder of the values and particular values for Structural Formulas (III)-(VII) and Structural Formulas (IIIa)-(VIIa) are as described for the fifth set of values for Structural Formulas (III)-(VII) and Structural Formulas (IIIa)-(VIIa).

A tenth set of values for Structural Formulas (III)-(VII) and Structural Formulas (IIIa)-(VIIa) is provided in the following paragraphs:

G is OH, NH₂ or NHR^{e};

R^{e} is a) (C₁-C₆)alkyl, halo(C₁-C₆)alkyl, (C₄-C₁₀)cycloalkylalkyl, (C₁-C₅)alkoxy(C₁-C₅)alkyl, or aminocarbonyl(C₁-C₆)alkyl or b) phenyl(C₁-C₂)alkyl optionally substituted with 1 to 3 groups independently selected from: fluorine, chlorine, cyano, (C₁-C₃)alkyl, halo(C₁-C₃)alkyl, (C₁-C₃)alkoxy, and halo(C₁-C₃)alkoxy; or c) R⁵ and R^{e} together are —CH₂—, —(CH₂)₂—, —(CH₂)₃—, or —(CH₂)₄—, optionally substituted with 1 or 2 groups independently selected from fluorine, (C₁-C₈)alkyl, halo(C₁-C₈)alkyl, (C₃-C₆)cycloalkyl, halo(C₃-C₆)cycloalkyl, hydroxy(C₃-C₆)cycloalkyl, (C₃-C₆)cycloalkyl(C₁-C₂)alkyl, halo(C₃-C₆)cycloalkyl(C₁-C₂)alkyl, hydroxy(C₃-C₆)cycloalkyl(C₁-C₂)alkyl, (C₁-C₈)alkoxy, halo(C₁-C₈)alkoxy, (C₃-C₆)cycloalkoxy, halo(C₃-C₆)cycloalkoxy, and heterocyclyl;

and the remainder of the values and particular values for Structural Formulas (III)-(VII) and Structural Formulas (IIIa)-(VIIa) are as described for the sixth set of values for Structural Formulas (III)-(VII) and Structural Formulas (IIIa)-(VIIa).

In a fourth specific embodiment, the aspartic protease inhibitor of the invention is represented by a structural formula selected from Structural Formulas (VIII)-(XII) and Structural Formulas (VIIIa)-(XIIa), or an enantiomer, diastereomer or a pharmaceutically acceptable salt thereof:

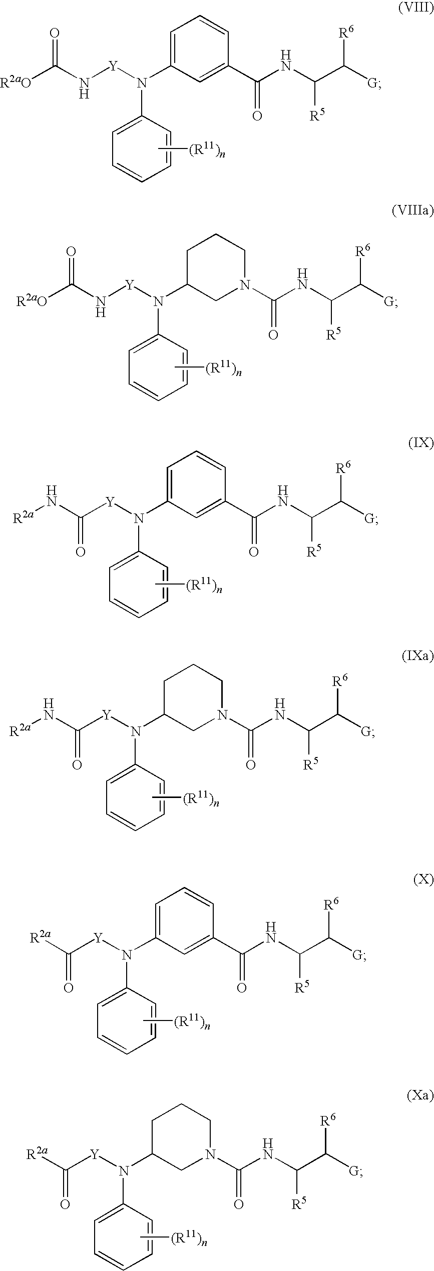

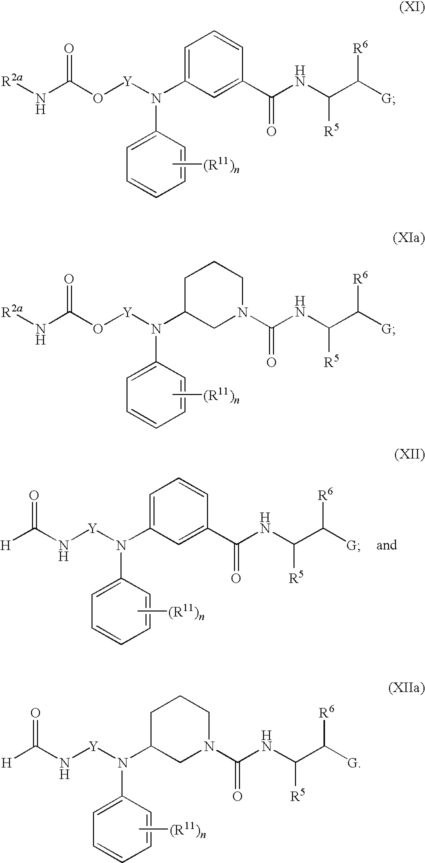

Values and particular values for the variables in Structural Formulas (VIII)-(XII) and Structural Formulas (VIIIa)-(XIIa) are as provided for in Structural Formula (I) above.

A first set of values for Structural Formulas (VIII)-(XII) and Structural Formulas (VIIIa)-(XIIa) is described in the following paragraphs:

n is 0, 1, 2, or 3;

R¹¹ is fluorine, chlorine, bromine, cyano, nitro, (C₁-C₆)alkyl, (C₃-C₆)cycloalkyl, (C₄-C₇)cycloalkylalkyl, (C₂-C₆)alkenyl, (C₅-C₇)cycloalkylalkenyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, halo(C₂-C₆)alkenyl, halo(C₃-C₆)alkynyl, halo(C₅-C₇)-cycloalkylalkynyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy and (C₁-C₆)alkanesulfonyl; or 2) phenyl, heteroaryl, phenoxy, heteroaryloxy, phenylthio, heteroarylthio, benzyl, heteroarylmethyl, benzyloxy and heteroarylmethoxy, each optionally substituted with 1 to 3 groups independently selected from: fluorine, chlorine, cyano, (C₁-C₃)alkyl, halo(C₁-C₃)alkyl, (C₁-C₃)-alkoxy, and halo(C₁-C₃)alkoxy, and aminocarbonyl; and the values and particular values for the remainder of the variables in Structural Formulas (VIII)-(XII) and Structural Formulas (VIIIa)-(XIIa) are as provided for Structural Formula (I) above.

A second set of values for Structural Formulas (VIII)-(XII) and Structural Formulas (VIIIa)-(XIIa) is provided in the following paragraphs:

G is OH, NH₂ or NHR^{e};

R^{e} is a) (C₁-C₆)alkyl, halo(C₁-C₆)alkyl, (C₄-C₁₀)cycloalkylalkyl, (C₁-C₅)alkoxy(C₁-C₅)alkyl, or aminocarbonyl(C₁-C₆)alkyl or b) phenyl(C₁-C₂)alkyl optionally substituted with 1 to 3 groups independently selected from: fluorine, chlorine, cyano, (C₁-C₃)alkyl, halo(C₁-C₃)alkyl, (C₁-C₃)alkoxy, and halo(C₁-C₃)alkoxy; or c) R⁵ and R^{e} together are —CH₂—, —(CH₂)₂—, —(CH₂)₃—, or —(CH₂)₄—, optionally substituted with 1 or 2 groups independently selected from fluorine, (C₁-C₈)alkyl, halo(C₁-C₈)alkyl, (C₃-C₆)cycloalkyl, halo(C₃-C₆)cycloalkyl, hydroxy(C₃-C₆)cycloalkyl, (C₃-C₆)cycloalkyl(C₁-C₂)alkyl, halo(C₃-C₆)cycloalkyl(C₁-C₂)alkyl, hydroxy(C₃-C₆)cycloalkyl(C₁-C₂)alkyl, (C₁-C₈)alkoxy, halo(C₁-C₈)alkoxy, (C₃-C₆)cycloalkoxy, halo(C₃-C₆)cycloalkoxy, and heterocyclyl;

and the remainder of the values and particular values for Structural Formulas (VIII)-(XII) and Structural Formulas (VIIIa)-(XIIa) are as described for the first set of values for Structural Formulas (VIII)-(XII) and Structural Formulas (VIIIa)-(XIIa).

A third set of values for the compounds represented by Structural Formulas (VIII)-(XII) and Structural Formulas (VIIIa)-(XIIa) is provide in the following paragraphs:

R⁵ is (C₁-C₇)alkyl, halo(C₁-C₇)alkyl, hydroxy(C₁-C₇)alkyl, cyclohexylmethyl, halocyclohexylmethyl, hydroxylated cyclohexylmethyl, 2-(cyclohexyl)ethyl, (C₁-C₂)alkyl cyclohexylmethyl, di(C₁-C₂)alkyl cyclohexylmethyl, hydroxylated (C₁-C₂)alkyl cyclohexylmethyl, hydroxylated di(C₁-C₂)alkylcyclohexylmethyl, (3-noradamantyl)methyl, (tetrahydropyranyl)methyl, or oxepanyl methyl;

R⁶ is —H or methyl;

G is NH₂ or NHR^{e};

R^{e} is methyl or R⁵ and R^{e} together are —(CH₂)₃— optionally substituted with C₁-C₄ alkyl or cyclohexyl; and

the remainder of the values and particular values for Structural Formulas (VIII)-(XII) and Structural Formulas (VIIIa)-(XIIa) are as described for the first set of values for Structural Formulas (VIII)-(XII) and Structural Formulas (VIIIa)-(XIIa).

A fourth set of values for the compounds of Structural Formulas (VIII)-(XII) and Structural Formulas (VIIIa)-(XIIa) is described in the following paragraphs:

R⁶ is (C₁-C₇)alkyl, halo(C₁-C₇)alkyl, hydroxy(C₁-C₇)alkyl, cyclohexylmethyl, halocyclohexylmethyl, hydroxylated cyclohexylmethyl, 2-(cyclohexyl)ethyl, (C₁-C₂)alkyl cyclohexylmethyl, di(C₁-C₂)alkyl cyclohexylmethyl, hydroxylated (C₁-C₂)alkyl cyclohexylmethyl, hydroxylated di(C₁-C₂)alkylcyclohexylmethyl, (3-noradamantyl)methyl, (tetrahydropyranyl)methyl, or oxepanyl methyl;

R⁵ is —H or methyl;

G is NH₂ or NHR^{e};

R^{e} is methyl or R⁵ and R^{e} together are —(CH₂)₃— optionally substituted with C₁-C₄ alkyl or cyclohexyl; and

the remainder of the values and particular values for Structural Formulas (VIII)-(XII) and Structural Formulas (VIIIa)-(XIIa) are as described for the first set of values for Structural Formulas (VIII)-(XII) and Structural Formulas (VIIIa)-(XIIa).

A fifth set of values for the compounds represented Structural Formulas (VIII)-(XII) and Structural Formulas (VIIIa)-(XIIa) is provided in the following paragraphs

R^{2a} is methyl or ethyl;

R¹¹ is chloro, fluoro or methyl; and the remainder of the values and particular values for Structural Formulas (VIII)-(XII) and Structural Formulas (VIIIa)-(XIIa) are as described in the third set of values for Structural Formulas (VIII)-(XII) and Structural Formulas (VIIIa)-(XIIa)

A sixth set of values for the compounds represented Structural Formulas (VIII)-(XII) and Structural Formulas (VIIIa)-(XIIa) is provided in the following paragraphs

R^{2a} is methyl or ethyl;

R¹¹ is chloro, fluoro or methyl; and

the remainder of the values and particular values for Structural Formulas (VIII)-(XII) and Structural Formulas (VIIIa)-(XIIa) are as described in the fourth set of values for Structural Formulas (VIII)-(XII) and Structural Formulas (VIIIa)-(XIIa).

A seventh set of values for Structural Formulas (VIII)-(XII) and Structural Formulas (VIIIa)-(XIIa) is described in the following paragraphs: one of R⁵ and R⁶ is H or methyl and the other is a) H, (C₁-C₁₀)alkyl, (C₄-C₁₀)cycloalkylalkyl, halo(C₁-C₁₀)alkyl, hydroxy(C₁₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁₀)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₅)alkyl, or saturated heterocyclyl(C₁-C₃)alkyl; or b) phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy; and

the remainder of the values and particular values for Structural Formulas (VIII)-(XII) and Structural Formulas (VIIIa)-(XIIa) are as described in the first set of values for Structural Formulas (VIII)-(XII) and Structural Formulas (VIIIa)-(XIIa).

A eighth set of values for Structural Formulas (VIII)-(XII) and Structural Formulas (VIIIa)-(XIIa) is described in the following paragraphs:

R⁶ is H or methyl and R⁵ is selected from a) H, (C₁-C₁₀)alkyl, (C₄-C₁₀)cycloalkylalkyl, halo(C₁-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁₀)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₅)alkyl, or saturated heterocyclyl(C₁-C₃)alkyl; or b) phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy; and

the remainder of the values and particular values for Structural Formulas (VIII)-(XII) and Structural Formulas (VIIIa)-(XIIa) are as described for the first set of values for Structural Formulas (VIII)-(XII) and Structural Formulas (VIIIa)-(XIIa).

A ninth set of values for Structural Formulas (VIII)-(XII) and Structural Formulas (VIIIa)-(XIIa) is described in the following paragraphs:

R⁵ is H or methyl and R⁶ is selected from a) H, (C₁-C₁₀)alkyl, (C₄-C₁₀)cycloalkylalkyl, halo(C₁-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₅)alkyl, or saturated heterocyclyl(C₁-C₃)alkyl; or b) phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy; and

the remainder of the values and particular values for Structural Formulas (VIII)-(XII) and Structural Formulas (VIIIa)-(XIIa) are as described for the first set of values in Structural Formulas (VIII)-(XII) and Structural Formulas (VIIIa)-(XIIa).

A tenth set of values for Structural Formulas (VIII)-(XII) and Structural Formulas (VIIIa)-(XIIa) is provided in the following paragraphs:

G is OH, NH₂ or NHR^{e};

R^{e} is a) (C₁-C₆)alkyl, halo(C₁-C₆)alkyl, (C₄-C₁₀)cycloalkylalkyl, (C₁-C₅)alkoxy(C₁-C₅)alkyl, or aminocarbonyl(C₁-C₆)alkyl or b) phenyl(C₁-C₂)alkyl optionally substituted with 1 to 3 groups independently selected from: fluorine, chlorine, cyano, (C₁-C₃)alkyl, halo(C₁-C₃)alkyl, (C₁-C₃)alkoxy, and halo(C₁-C₃)alkoxy; or c) R⁵ and R^{e} together are —CH₂—, —(CH₂)₂—, —(CH₂)₃—, or —(CH₂)₄—, optionally substituted with 1 or 2 groups independently selected from fluorine, (C₁-C₈)alkyl, halo(C₁-C₈)alkyl, (C₃-C₆)cycloalkyl, halo(C₃-C₆)cycloalkyl, hydroxy(C₃-C₆)cycloalkyl, (C₃-C₆)cycloalkyl(C₁-C₂)alkyl, halo(C₃-C₆)cycloalkyl(C₁-C₂)alkyl, hydroxy(C₃-C₆)cycloalkyl(C₁-C₂)alkyl, (C₁-C₈)alkoxy, halo(C₁-C₈)alkoxy, (C₃-C₆)cycloalkoxy, halo(C₃-C₆)cycloalkoxy, and heterocyclyl;

and the remainder of the values and particular values for Structural Formulas (VIII)-(XII) and Structural Formulas (VIIIa)-(XIIa) are as described for the seventh set of values for Structural Formulas (VIII)-(XII) and Structural Formulas (VIIIa)-(XIIa).

A eleventh set of values for Structural Formulas (VIII)-(XII) and Structural Formulas (VIIIa)-(XIIa) is provided in the following paragraphs:

G is OH, NH₂ or NHR^{e};

R^{e} is a) (C₁-C₆)alkyl, halo(C₁-C₆)alkyl, (C₄-C₁₀)cycloalkylalkyl, (C₁-C₅)alkoxy(C₁-C₅)alkyl, or aminocarbonyl(C₁-C₆)alkyl or b) phenyl(C₁-C₂)alkyl optionally substituted with 1 to 3 groups independently selected from: fluorine, chlorine, cyano, (C₁-C₃)alkyl, halo(C₁-C₃)alkyl, (C₁-C₃)alkoxy, and halo(C₁-C₃)alkoxy; or c) R⁵ and R^{e} together are —CH₂—, —(CH₂)₂—, —(CH₂)₃—, or —(CH₂)₄—, optionally substituted with 1 or 2 groups independently selected from fluorine, (C₁-C₈)alkyl, halo(C₁-C₈)alkyl, (C₃-C₆)cycloalkyl, halo(C₃-C₆)cycloalkyl, hydroxy(C₃-C₆)cycloalkyl, (C₃-C₆)cycloalkyl(C₁-C₂)alkyl, halo(C₃-C₆)cycloalkyl(C₁-C₂)alkyl, hydroxy(C₃-C₆)cycloalkyl(C₁-C₂)alkyl, (C₁-C₈)alkoxy, halo(C₁-C₈)alkoxy, (C₃-C₆)cycloalkoxy, halo(C₃-C₆)cycloalkoxy, and heterocyclyl;

and the remainder of the values and particular values for Structural Formulas (VIII)-(XII) and Structural Formulas (VIIIa)-(XIIa) are as described for the eighth set of values for Structural Formulas (VIII)-(XII) and Structural Formulas (VIIIa)-(XIIa).

A twelfth set of values for Structural Formulas (VIII)-(XII) and Structural Formulas (VIIIa)-(XIIa) is provided in the following paragraphs:

G is OH, NH₂ or NHR^{e};

R^{e} is a) (C₁-C₆)alkyl, halo(C₁-C₆)alkyl, (C₄-C₁₀)cycloalkylalkyl, (C₁-C₅)alkoxy(C₁-C₅)alkyl, or aminocarbonyl(C₁-C₆)alkyl or b) phenyl(C₁-C₂)alkyl optionally substituted with 1 to 3 groups independently selected from: fluorine, chlorine, cyano, (C₁-C₃)alkyl, halo(C₁-C₃)alkyl, (C₁-C₃)alkoxy, and halo(C₁-C₃)alkoxy; or c) R⁵ and R^{e} together are —CH₂—, —(CH₂)₂—, —(CH₂)₃—, or —(CH₂)₄—, optionally substituted with 1 or 2 groups independently selected from fluorine, (C₁-C₈)alkyl, halo(C₁-C₈)alkyl, (C₃-C₆)cycloalkyl, halo(C₃-C₆)cycloalkyl, hydroxy(C₃-C₆)cycloalkyl, (C₃-C₆)cycloalkyl(C₁-C₂)alkyl, halo(C₃-C₆)cycloalkyl(C₁-C₂)alkyl, hydroxy(C₃-C₆)cycloalkyl(C₁-C₂)alkyl, (C₁-C₈)alkoxy, halo(C₁-C₈)alkoxy, (C₃-C₆)cycloalkoxy, halo(C₃-C₆)cycloalkoxy, and heterocyclyl;

and the remainder of the values and particular values for Structural Formulas (VIII)-(XII) and Structural Formulas (VIIIa)-(XIIa) are as described for the ninth set of values for Structural Formulas (VIII)-(XII) and Structural Formulas (VIIIa)-(XIIa).

In a fifth specific embodiment, the aspartic protease inhibitor of the invention is represented by a structural formula selected from Structural Formulas (XIII)-(XVII) and Structural Formulas (XIIIa)-(XVIIa), or enantiomers, diastereomers or pharmaceutically acceptable salts thereof:

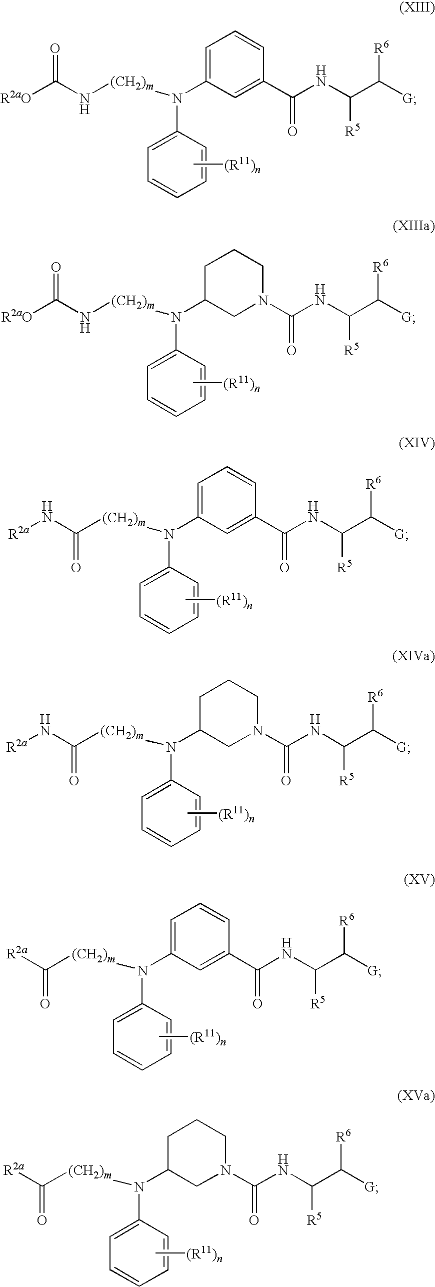

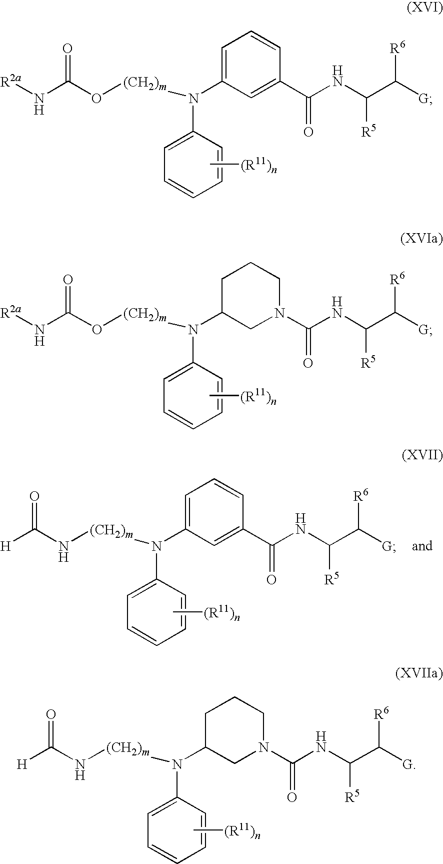

A first set of values for Structural Formulas (XIII)-(XVII) and Structural Formulas (XIIIa)-(XVIIa) is described in the following paragraphs:

m is 2 or 3; and

the remainder of the values and particular values for Structural Formulas (XIII)-(XVII) and Structural Formulas (XIIIa)-(XVIIa) are as described for the first set of values for Structural Formulas (VIII)-(XII) and Structural formulas (VIIIa)-(XIIa).

A second set of values for Structural Formulas (XIII)-(XVII) and Structural Formulas (XIIIa)-(XVIIa) is described in the following paragraphs:

m is 2 or 3; and

the remainder of the values and particular values for Structural Formulas (XIII)-(XVII) and Structural Formulas (XIIIa)-(XVIIa) are as described for the second set of values for Structural Formulas (VIII)-(XII) and Structural formulas (VIIIa)-(XIIa).

A third set of values for Structural Formulas (XIII)-(XVII) and Structural Formulas (XIIIa)-(XVIIa) is described in the following paragraphs:

m is 2 or 3; and

the remainder of the values and particular values for Structural Formulas (XIII)-(XVII) and Structural Formulas (XIIIa)-(XVIIa) are as described for the third set of values for Structural Formulas (VIII)-(XII) and Structural formulas (VIIIa)-(XIIa).

A fourth set of values for Structural Formulas (XIII)-(XVII) and Structural Formulas (XIIIa)-(XVIIa) is described in the following paragraphs:

m is 2 or 3; and

the remainder of the values and particular values for Structural Formulas (XIII)-(XVII) and Structural Formulas (XIIIa)-(XVIIa) are as described for the fourth set of values for Structural Formulas (VIII)-(XII) and Structural formulas (VIIIa)-(XIIa).

A fifth set of values for Structural Formulas (XIII)-(XVII) and Structural Formulas (XIIIa)-(XVIIa) is described in the following paragraphs:

m is 2 or 3; and

the remainder of the values and particular values for Structural Formulas (XIII)-(XVII) and Structural Formulas (XIIIa)-(XVIIa) are as described for the fifth set of values for Structural Formulas (VIII)-(XII) and Structural formulas (VIIIa)-(XIIa).

A sixth set of values for Structural Formulas (XIII)-(XVII) and Structural Formulas (XIIIa)-(XVIIa) is described in the following paragraphs:

m is 2 or 3; and

the remainder of the values and particular values for Structural Formulas (XIII)-(XVII) and Structural Formulas (XIIIa)-(XVIIa) are as described for the sixth set of values for Structural Formulas (VIII)-(XII) and Structural formulas (VIIIa)-(XIIa).

A seventh set of values for Structural Formulas (XIII)-(XVII) and Structural Formulas (XIIIa)-(XVIIa) is described in the following paragraphs:

m is 2 or 3; and the remainder of the values and particular values for Structural Formulas (XIII)-(XVII) and Structural Formulas (XIIIa)-(XVIIa) are as described for the seventh set of values for Structural Formulas (VIII)-(XII) and Structural formulas (VIIIa)-(XIIa).

An eighth set of values for Structural Formulas (XIII)-(XVII) and Structural Formulas (XIIIa)-(XVIIa) is described in the following paragraphs:

m is 2 or 3; and

the remainder of the values and particular values for Structural Formulas (XIII)-(XVII) and Structural Formulas (XIIIa)-(XVIIa) are as described for the eighth set of values for Structural Formulas (VIII)-(XII) and Structural Formulas (VIIIa)-(XIIa).

A ninth set of values for Structural Formulas (XIII)-(XVII) and Structural Formulas (XIIIa)-(XVIIa) is described in the following paragraphs:

m is 2 or 3; and

the remainder of the values and particular values for Structural Formulas (XIII)-(XVII) and Structural Formulas (XIIIa)-(XVIIa) are as described for the ninth set of values for Structural Formulas (VIII)-(XII) and Structural Formulas (VIIIa)-(XIIa).

A tenth set of values for Structural Formulas (XIII)-(XVII) and Structural Formulas (XIIIa)-(XVIIa) is described in the following paragraphs:

m is 2 or 3; and

the remainder of the values and particular values for Structural Formulas (XIII)-(XVII) and Structural Formulas (XIIIa)-(XVIIa) are as described for the tenth set of values for Structural Formulas (VIII)-(XII) and Structural formulas (VIIIa)-(XIIa).

A eleventh set of values for Structural Formulas (XIII)-(XVII) and Structural Formulas (XIIIa)-(XVIIa) is described in the following paragraphs:

m is 2 or 3; and

the remainder of the values and particular values for Structural Formulas (XIII)-(XVII) and Structural Formulas (XIIIa)-(XVIIa) are as described for the eleventh set of values for Structural Formulas (VIII)-(XII) and Structural formulas (VIIIa)-(XIIa).

A twelfth set of values for Structural Formulas (XIII)-(XVII) and Structural Formulas (XIIIa)-(XVIIa) is described in the following paragraphs:

m is 2 or 3; and

the remainder of the values and particular values for Structural Formulas (XIII)-(XVII) and Structural Formulas (XIIIa)-(XVIIa) are as described for the twelfth set of values for Structural Formulas (VIII)-(XII) and Structural formulas (VIIIa)-(XIIa).

In a sixth embodiment, the aspartic protease inhibitor is represented by Structural Formula (XVIII) and enantiomers, diastereomer and pharmaceutically acceptable salts thereof:

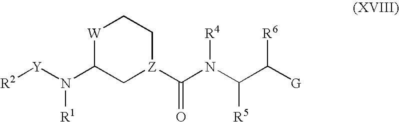

A first set of values for Structural Formula (XVIII) is provided in the following paragraphs:

W═CH₂ or O and Z═CH or N

one of R⁵ and R⁶ is —H or methyl and the other is as described for Structural Formula (I); and

the remainder of the values and particular values for Structural Formula (XVIII) are as described for Structural Formula (I).

A second set of values for Structural Formula (XVIII) is provided in the following paragraphs:

W═CH₂ or O and Z═CH or N;

R⁶ is —H or methyl; and

the remainder of the values and particular values for Structural Formula (XVIII) are as described for Structural Formula (I).

A third set of values for Structural Formula (XVIII) is provided in the following paragraphs:

W═CH₂ or O and Z═CH or N;

R⁵ is —H or methyl; and

the remainder of the values and particular values for Structural Formula (XVIII) are as described for Structural Formula (I).

A fourth set of values for Structural Formula (XVIII) is provided in the following paragraphs:

W═CH₂ or O and Z═CH or N;

one of R⁵ and R⁶ is H or methyl and the other is selected from a) H, (C₁-C₁₀)alkyl, (C₄-C₁₀)cycloalkylalkyl, halo(C₁-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁₀)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₅)alkyl, or saturated heterocyclyl(C₁-C₃)alkyl; or b) phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy; and

the remainder of the values and particular values for Structural Formula (XVIII) are as described for Structural Formula (I).

A fifth set of values for Structural Formula (XVIII) is provided in the following paragraphs:

W═CH₂ or O and Z═CH or N;

R⁶ is H or methyl and R^{s} is selected from a) H, (C₁-C₁₀)alkyl, (C₄-C₁₀)cycloalkylalkyl, halo(C₁-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁₀)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₅)alkyl, or saturated heterocyclyl(C₁-C₃)alkyl; or b) phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy; and

the remainder of the values and particular values for Structural Formula (XVIII) are as described for Structural Formula (I).

A sixth set of values for Structural Formula (XVIII) is provided in the following paragraphs:

W═CH₂ or O and Z═CH or N;

R^{s} is H or methyl and R⁶ is selected from a) H, (C₁-C₁₀)alkyl, (C₄-C₁₀)cycloalkylalkyl, halo(C₁-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁₀)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₄)alkyl, or saturated heterocyclyl(C₁-C₃)alkyl; or b) phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy; and

the remainder of the values and particular values for Structural Formula (XVIII) are as described for Structural Formula (I).

A seventh set of values for Structural Formula (XVIII) is provided in the following paragraphs:

W═CH₂ or O and Z═CH or N

G is OH, NH₂ or NHR^{e};

R^{e} is a) (C₁-C₆)alkyl, halo(C₁-C₆)alkyl, (C₄-C₁₀)cycloalkylalkyl, (C₁-C₅)alkoxy(C₁-C₅)alkyl, or aminocarbonyl(C₁-C₆)alkyl or b) phenyl(C₁-C₂)alkyl optionally substituted with 1 to 3 groups independently selected from: fluorine, chlorine, cyano, (C₁-C₃)alkyl, halo(C₁-C₃)alkyl, (C₁-C₃)alkoxy, and halo(C₁-C₃)alkoxy; or c) R⁵ and R^{e} together are —CH₂—, —(CH₂)₂—, —(CH₂)₃—, or —(CH₂)₄—, optionally substituted with 1 or 2 groups independently selected from fluorine, (C₁-C₈)alkyl, halo(C₁-C₈)alkyl, (C₃-C₆)cycloalkyl, halo(C₃-C₆)cycloalkyl, hydroxy(C₃-C₆)cycloalkyl, (C₃-C₆)cycloalkyl(C₁-C₂)alkyl, halo(C₃-C₆)cycloalkyl(C₁-C₂)alkyl, hydroxy(C₃-C₆)cycloalkyl(C₁-C₂)alkyl, (C₁-C₈)alkoxy, halo(C₁-C₈)alkoxy, (C₃-C₆)cycloalkoxy, halo(C₃-C₆)cycloalkoxy, and heterocyclyl;

and the remainder of the values and particular values for Structural Formula (XVIII) are as described for Structural Formula (I).

A eighth set of values for Structural Formula (XVIII) is provided in the following paragraphs:

G is OH, NH₂ or NHR^{e};

R^{e} is a) (C₁-C₆)alkyl, halo(C₁-C₆)alkyl, (C₄-C₁₀)cycloalkylalkyl, (C₁-C₅)alkoxy(C₁-C₅)alkyl, or aminocarbonyl(C₁-C₆)alkyl or b) phenyl(C₁-C₂)alkyl optionally substituted with 1 to 3 groups independently selected from: fluorine, chlorine, cyano, (C₁-C₃)alkyl, halo(C₁-C₃)alkyl, (C₁-C₃)alkoxy, and halo(C₁-C₃)alkoxy; or c) R⁵ and R^{e} together are —CH₂—, —(CH₂)₂—, —(CH₂)₃—, or —(CH₂)₄—, optionally substituted with 1 or 2 groups independently selected from fluorine, (C₁-C₈)alkyl, halo(C₁-C₈)alkyl, (C₃-C₆)cycloalkyl, halo(C₃-C₆)cycloalkyl, hydroxy(C₃-C₆)cycloalkyl, (C₃-C₆)cycloalkyl(C₁-C₂)alkyl, halo(C₃-C₆)cycloalkyl(C₁-C₂)alkyl, hydroxy(C₃-C₆)cycloalkyl(C₁-C₂)alkyl, (C₁-C₈)alkoxy, halo(C₁-C₈)alkoxy, (C₃-C₆)cycloalkoxy, halo(C₃-C₆)cycloalkoxy, and heterocyclyl; and the remainder of the values and particular values for Structural Formula (XVIII) are as described for the fourth set of values for Structural Formula (XVIII).

A ninth set of values for Structural Formula (XVIII) is provided in the following paragraphs:

G is OH, NH₂ or NHR^{e};

R^{e} is a) (C₁-C₆)alkyl, halo(C₁-C₆)alkyl, (C₄-C₁₀)cycloalkylalkyl, (C₁-C₅)alkoxy(C₁-C₈)alkyl, or aminocarbonyl(C₁-C₆)alkyl or b) phenyl(C₁-C₂)alkyl optionally substituted with 1 to 3 groups independently selected from: fluorine, chlorine, cyano, (C₁-C₃)alkyl, halo(C₁-C₃)alkyl, (C₁-C₃)alkoxy, and halo(C₁-C₃)alkoxy; or c) R⁵ and R^{e} together are —CH₂—, —(CH₂)₂—, —(CH₂)₃—, or —(CH₂)₄—, optionally substituted with 1 or 2 groups independently selected from fluorine, (C₁-C₈)alkyl, halo(C₁-C₈)alkyl, (C₃-C₆)cycloalkyl, halo(C₃-C₆)cycloalkyl, hydroxy(C₃-C₆)cycloalkyl, (C₃-C₆)cycloalkyl(C₁-C₂)alkyl, halo(C₃-C₆)cycloalkyl(C₁-C₂)alkyl, hydroxy(C₃-C₆)cycloalkyl(C₁-C₂)alkyl, (C₁-C₈)alkoxy, halo(C₁-C₈)alkoxy, (C₃-C₆)cycloalkoxy, halo(C₃-C₆)cycloalkoxy, and heterocyclyl;

and the remainder of the values and particular values for Structural Formula (XVIII) are as described for the fifth set of values for Structural Formula (XVIII).

A tenth set of values for Structural Formula (XVIII) is provided in the following paragraphs:

G is OH, NH₂ or NHR^{e};

R^{e} is a) (C₁-C₆)alkyl, halo(C₁-C₆)alkyl, (C₄-C₁₀)cycloalkylalkyl, (C₁-C₅)alkoxy(C₁-C₅)alkyl, or aminocarbonyl(C₁-C₆)alkyl or b) phenyl(C₁-C₂)alkyl optionally substituted with 1 to 3 groups independently selected from: fluorine, chlorine, cyano, (C₁-C₃)alkyl, halo(C₁-C₃)alkyl, (C₁-C₃)alkoxy, and halo(C₁-C₃)alkoxy; or c) R⁵ and R^{e} together are —CH₂—, —(CH₂)₂—, —(CH₂)₃—, or —(CH₂)₄—, optionally substituted with 1 or 2 groups independently selected from fluorine, (C₁-C₈)alkyl, halo(C₁-C₈)alkyl, (C₃-C₆)cycloalkyl, halo(C₃-C₆)cycloalkyl, hydroxy(C₃-C₆)cycloalkyl, (C₃-C₆)cycloalkyl(C₁-C₂)alkyl, halo(C₃-C₆)cycloalkyl(C₁-C₂)alkyl, hydroxy(C₃-C₆)cycloalkyl(C₁-C₂)alkyl, (C₁-C₈)alkoxy, halo(C₁-C₈)alkoxy, (C₃-C₆)cycloalkoxy, halo(C₃-C₆)cycloalkoxy, and heterocyclyl;

and the remainder of the values and particular values for Structural Formula (XVIII) are as described for the sixth set of values for Structural Formula (XVIII).

In a seventh embodiment, the aspartic protease inhibitor is represented by Structural Formulas (XIX), (XX) and (XXI) and enantiomers, diastereomers and pharmaceutically acceptable salts thereof:

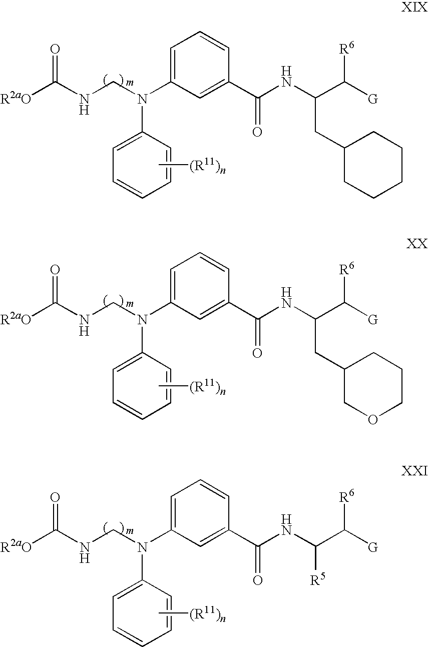

Values and Particular values for Structural Formulas XIX, XX and XXI are as described for the first set of values for Structural Formulas XIII-XVII and XIIIa-XVIIa.

Alternatively, values and particular values for Structural Formulas XIX, XX and XXI are as described for the second set of values for Structural Formulas XIII-XVII and XIIIa-XVIIa.

Alternatively, values and particular values for Structural Formulas XIX, XX and XXI are as described for the third set of values for Structural Formulas XIII-XVII and XIIIa-XVIIa.

Alternatively, values and particular values for Structural Formulas XIX, XX and XXI are as described for the fourth set of values for Structural Formulas XIII-XVII and XIIIa-XVIIa.

Alternatively, values and particular values for Structural Formulas XIX, XX and XXI are as described for the fifth set of values for Structural Formulas XIII-XVII and XIIIa-XVIIa.

Alternatively, values and particular values for Structural Formulas XIX, XX and XXI are as described for the sixth set of values for Structural Formulas XIII-XVII and XIIIa-XVIIa.

Alternatively, values and particular values for Structural Formulas XIX, XX and XXI are as described for the seventh set of values for Structural Formulas XIII-XVII and XIIIa-XVIIa.

Alternatively, values and particular values for Structural Formulas XIX, XX and XXI are as described for the eighth set of values for Structural Formulas XIII-XVII and XIIIa-XVIIa.

Alternatively, values and particular values for Structural Formulas XIX, XX and XXI are as described for ninth set of values for Structural Formulas XIII-XVII and XIIIa-XVIIa.

Alternatively, values and particular values for Structural Formulas XIX, XX and XXI are as described for the tenth set of values for Structural Formulas XIII-XVII and XIIIa-XVIIa.

Alternatively, values and particular values for Structural Formulas XIX, XX and XXI are as described for the eleventh set of values for Structural Formulas XIII-XVII and XIIIa-XVIIa.

Alternatively, values and particular values for Structural Formulas XIX, XX and XXI are as described for the twelfth set of values for Structural Formulas XIII-XVII and XIIIa-XVIIa.

Another embodiment of the invention is each of the following compounds and their enantiomers, diastereomers, and salts:

| Cpd. | |
|---|---|
| No. | Name |
| I-1 | methyl 3-((3-chlorophenyl)(1-(1-cyclohexyl-3- |
| | (methylamino)propan-2-ylcarbamoyl)piperidin-3- |
| | yl)amino)propylcarbamate |
| I-2 | methyl 3-((5-chloro-2-methylphenyl)(1-(1-cyclohexyl-3- |
| | (methylamino)propan-2-ylcarbamoyl)piperidin-3- |
| | yl)amino)propylcarbamate |
| I-3 | methyl 3-((3-chlorophenyl)(1-(1-(methylamino)-3-(-tetrahydro-2H- |
| | pyran-3-yl)propan-2-ylcarbamoyl)piperidin-3- |
| | yl)amino)propylcarbamate |
| I-4 | methyl 3-((3-chlorophenyl)(3-(1-cyclohexyl-3- |
| | (methylamino)propan-2- |
| | ylcarbamoyl)phenyl)amino)propylcarbamate |

or a diastereomer, enantiomer or salt thereof.
The following are compounds of the invention, especially their pharmaceutically acceptable salts:

| No. | Structure | Name |
|---|---|---|
| I-1a | 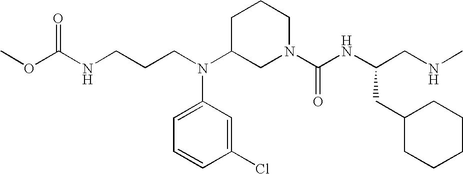 | methyl 3-((3-chlorophenyl)(1-((S)-1- cyclohexyl-3-(methylamino)propan-2- ylcarbamoyl)piperidin-3- yl)amino)propylcarbamate |
| I-2a | 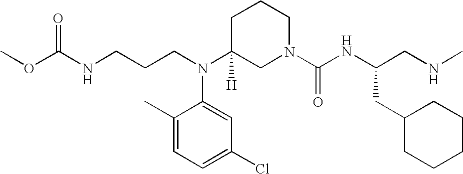 | methyl 3-((5-chloro-2- methylphenyl)((S)-1-((S)-1-cyclohexyl- 3-(methylamino)propan-2- ylcarbamoyl)piperidin-3- yl)amino)propylcarbamate |
| I-2b | 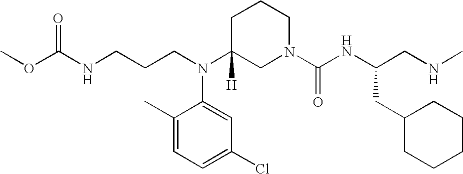 | methyl 3-((5-chloro-2- methylphenyl)((R)-1-((S)-1-cyclohexyl- 3-(methylamino)propan-2- ylcarbamoyl)piperidin-3- yl)amino)propylcarbamate |
| I-3a | 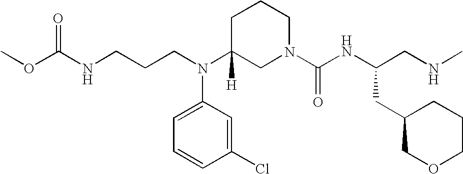 | methyl 3-((3-chlorophenyl)((R)-1-((S)-1- (methylamino)-3-((R)-tetrahydro-2H- pyran-3-yl)propan-2- ylcarbamoyl)piperidin-3- yl)amino)propylcarbamate |
| I-4a | 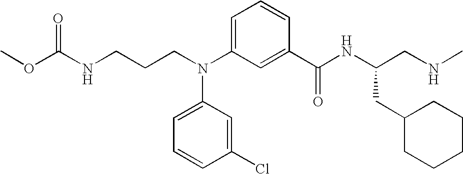 | (S)-methyl 3-((3-chlorophenyl)(3-(1- cyclohexyl-3-(methylamino)propan-2- ylcarbamoyl)phenyl)amino) propylcarbamate |

or a diastereomer, enantiomer or salt thereof.

The following, including pharmaceutically acceptable salts thereof, are preferred compounds of Formula I: I-1a and I-2b.

When any variable (e.g., aryl, heterocyclyl, R¹, R², etc.) occurs more than once in a compound, its definition on each occurrence is independent of any other occurrence.

“Alkyl” means a saturated aliphatic branched or straight-chain mono- or di-valent hydrocarbon radical having the specified number of carbon atoms. Thus, “(C₁-C₈)alkyl” means a radical having from 1-8 carbon atoms in a linear or branched arrangement. “(C₁-C₆)alkyl” includes methyl, ethyl, propyl, butyl, pentyl, and hexyl.

“Cycloalkyl” means a saturated aliphatic cyclic hydrocarbon radical having the specified number of carbon atoms. Thus, (C₃-C₇)cycloalkyl means a radical having from 3-7 carbon atoms arranged in a ring. (C₃-C₇)cycloalkyl includes cyclopropyl, cyclobutyl, cyclopentyl, cyclohexyl, and cycloheptyl.

Haloalkyl and halocycloalkyl include mono, poly, and perhaloalkyl groups where the halogens are independently selected from fluorine, chlorine, and bromine.

Saturated heterocyclic rings are 4-, 5-, 6-, and 7-membered heterocyclic rings containing 1 to 4 heteroatoms independently selected from N, O, and S, and include pyrrolidine, piperidine, tetrahydrofuran, tetrahydropyran, oxepane, tetrahydrothiophene, tetrahydrothiopyran, isoxazolidine, 1,3-dioxolane, 1,3-dithiolane, 1,3-dioxane, 1,4-dioxane, 1,3-dithiane, 1,4-dithiane, morpholine, thiomorpholine, thiomorpholine 1,1-dioxide, tetrahydro-2H-1,2-thiazine 1,1-dioxide, and isothiazolidine 1,1-dioxide. Oxo substituted saturated heterocyclic rings include tetrahydrothiophene 1-oxide, tetrahydrothiophene 1,1-dioxide, thiomorpholine 1-oxide, thiomorpholine 1,1-dioxide, tetrahydro-2H-1,2-thiazine 1,1-dioxide, and isothiazolidine 1,1-dioxide, pyrrolidin-2-one, piperidin-2-one, piperazin-2-one, and morpholin-2-one.

“Heteroaryl” means a monovalent heteroaromatic monocyclic or polycylic ring radical. Heteroaryl rings are 5- and 6-membered aromatic heterocyclic rings containing 1 to 4 heteroatoms independently selected from N, O, and S, and include furan, thiophene, pyrrole, imidazole, pyrazole, oxazole, isoxazole, thiazole, isothiazole, 1,2,3-triazole, 1,2,4-triazole, 1,3,4-oxadiazole, 1,2,5-thiadiazole, 1,2,5-thiadiazole 1-oxide, 1,2,5-thiadiazole 1,1-dioxide, 1,3,4-thiadiazole, pyridine, pyridine-N-oxide, pyrazine, pyrimidine, pyridazine, 1,2,4-triazine, 1,3,5-triazine, and tetrazole. Bicyclic heteroaryl rings are bicyclo[4.4.0] and bicyclo[4.3.0] fused ring systems containing 1 to 4 heteroatoms independently selected from N, O, and S, and include indolizine, indole, isoindole, benzo[b]furan, benzo[b]thiophene, indazole, benzimidazole, benzthiazole, purine, 4H-quinolizine, quinoline, isoquinoline, cinnoline, phtalazine, quinazoline, quinoxaline, 1,8-naphthyridine, and pteridine.

Bicycloalkyl rings are fused, bridged and spiro ring systems and include bicyclo[1.1.0]butane, bicyclo[1.2.0]pentane, bicyclo[2.2.0]hexane, bicyclo[3.2.0]heptane, bicyclo[3.3.0]octane, bicyclo[4.2.0]octane, bicyclo[2.2.1]heptane, bicyclo[2.2.2]octane, bicyclo[3.2.1]octane, bicyclo[3.2.2]nonane, bicyclo[3.3.1]nonane, bicyclo[3.3.2]decane and bicyclo[3.3.3]undecane, spiro[2.2]pentane, spiro[2.3]hexane, spiro[3.3]heptane, spiro[2.4]heptane, spiro[3.4]octane, and spiro[2.5]octane.

Tricycloalkyl rings are fused, bridged and spiro ring systems and include tricyclo[3.3.1.0^{3,7}]nonane (noradamantane) and tricyclo[3.3.1.1^{3,7}]decane (adamantane).

“Alkoxy” means an alkyl radical attached through an oxygen linking atom. “(C₁-C₄)-alkoxy” includes methoxy, ethoxy, propoxy, and butoxy.

“Aromatic” means an unsaturated cycloalkyl ring system.

“Aryl” means an aromatic monocyclic, or polycyclic ring system. Aryl systems include phenyl, naphthalenyl, fluorenyl, indenyl, azulenyl, and anthracenyl.

“Hetero” refers to the replacement of at least one carbon atom member in a ring system with at least one heteroatom selected from N, S, and O. A hetero ring may have 1, 2, 3, or 4 carbon atom members replaced by a heteroatom.

“Unsaturated ring” means a ring containing one or more double bonds and include cyclopentene, cyclohexene, cyclopheptene, cyclohexadiene, benzene, pyrroline, pyrazole, 4,5-dihydro-1H-imidazole, imidazole, 1,2,3,4-tetrahydropyridine, 1,2,3,6-tetrahydropyridine, pyridine and pyrimidine.

### Enantiomers, Diastereomers, and Salts

Certain compounds of Formula I may exist in various stereoisomeric or tautomeric forms. The invention encompasses all such forms, including active compounds in the form of essentially pure enantiomers, racemic mixtures, and tautomers, including forms those not depicted structurally.

The compounds of the invention may be present in the form of pharmaceutically acceptable salts. For use in medicines, the salts of the compounds of the invention refer to non-toxic “pharmaceutically acceptable salts.” Pharmaceutically acceptable salt forms include pharmaceutically acceptable acidic/anionic or basic/cationic salts.

Pharmaceutically acceptable acidic/anionic salts include, the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphospate, polygalacturonate, salicylate, stearate, subacetate, succinate, sulfate, tannate, tartrate, teoclate, tosylate, and triethiodide salts.

Salts of the disclosed compounds containing a carboxylic acid or other acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acid such as lysine and arginine.

When a disclosed compound or its pharmaceutically acceptable salt is named or depicted by structure, it is to be understood that solvates or hydrates of the compound or its pharmaceutically acceptable salts are also included. “Solvates” refer to crystalline forms wherein solvent molecules are incorporated into the crystal lattice during crystallization. Solvate may include water or nonaqueous solvents such as ethanol, isopropanol, DMSO, acetic acid, ethanolamine, and EtOAc. Solvates, wherein water is the solvent molecule incorporated into the crystal lattice, are typically referred to as “hydrates.” Hydrates include stoichiometric hydrates as well as compositions containing variable amounts of water.

When a disclosed compound or its pharmaceutically acceptable salt is named or depicted by structure, it is to be understood that the compound, including solvates thereof, may exist in crystalline forms, non-crystalline forms or a mixture thereof. The compound or its pharmaceutically acceptable salts or solvates may also exhibit polymorphism (i.e. the capacity to occur in different crystalline forms). These different crystalline forms are typically known as “polymorphs.” It is to be understood that when named or depicted by structure, the disclosed compound and its pharmaceutically acceptable salts, solvates or hydrates also include all polymorphs thereof. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in solidifying the compound. For example, changes in temperature, pressure, or solvent may result in different polymorphs. In addition, one polymorph may spontaneously convert to another polymorph under certain conditions.

It may be necessary and/or desirable during synthesis to protect sensitive or reactive groups on any of the molecules concerned. Representative conventional protecting groups are described in T. W. Greene and P. G. M. Wuts “Protective Groups in Organic Synthesis” John Wiley & Sons, Inc., New York 1999. Protecting groups may be added and removed using methods well known in the art.

The invention also includes various isomers and mixtures thereof. “Isomer” refers to compounds that have the same composition and molecular weight but differ in physical and/or chemical properties. The structural difference may be in constitution (geometric isomers) or in the ability to rotate the plane of polarized light (stereoisomers).

Certain of the disclosed aspartic protease inhibitors may exist in various stereoisomeric forms. Stereoisomers are compounds which differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms. The symbol “*” in a structural formula represents the presence of a chiral carbon center. “R” and “S” represent the configuration of substituents around one or more chiral carbon atoms. Thus, “R*” and “S*” denote the relative configurations of substituents around one or more chiral carbon atoms. When a chiral center is not defined as R or S, a mixture of both configurations is present.

“Racemate” or “racemic mixture” means a compound of equimolar quantities of two enantiomers, wherein such mixtures exhibit no optical activity; i.e., they do not rotate the plane of polarized light.

“Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration.

Atoms (other than H) attached to a carbocyclic ring may be in a cis or trans configuration. In the “cis” configuration, the substituents are on the same side in relationship to the plane of the ring; in the “trans” configuration, the substituents are on opposite sides in relationship to the plane of the ring. A mixture of “cis” and “trans” species is designated “cis/trans”.

The point at which a group or moiety is attached to the remainder of the compound or another group or moiety can be indicated by [symbol] which represents [symbol] , [symbol] or “—”.

“R,” “S,” “S*,” “R*,” “E,” “Z,” “cis,” and “trans,” indicate configurations relative to the core molecule.

The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

When the stereochemistry of a disclosed compound is named or depicted by structure, the named or depicted stereoisomer is at least 60%, 70%, 80%, 90%, 99% or 99.9% by weight pure relative to the other stereoisomers. When a single enantiomer is named or depicted by structure, the depicted or named enantiomer is at least 60%, 70%, 80%, 90%, 99% or 99.9% by weight optically pure. Percent optical purity by weight is the ratio of the weight of the enantiomer over the weight of the enantiomer plus the weight of its optical isomer.

When a disclosed compound is named or depicted by structure without indicating the stereochemistry, and the inhibitor has at least one chiral center, it is to be understood that the name or structure encompasses one enantiomer of inhibitor free from the corresponding optical isomer, a racemic mixture of the inhibitor and mixtures enriched in one enantiomer relative to its corresponding optical isomer.

When a disclosed aspartic protease inhibitor is named or depicted by structure without indicating the stereochemistry and has at least two chiral centers, it is to be understood that the name or structure encompasses a diastereomer free of other diastereomers, a pair of diastereomers free from other diastereomeric pairs, mixtures of diastereomers, mixtures of diastereomeric pairs, mixtures of diastereomers in which one diastereomer is enriched relative to the other diastereomer(s) and mixtures of diastereomeric pairs in which one diastereomeric pair is enriched relative to the other diastereomeric pair(s).

The compounds of the invention are useful for ameliorating or treating disorders or diseases in which decreasing the levels of aspartic protease products is effective in treating the disease state or in treating infections in which the infectious agent depends upon the activity of an aspartic protease. In hypertension elevated levels of angiotensin I, the product of renin catalyzed cleavage of angiotensinogen are present. Thus, the compounds of the invention can be used in the treatment of hypertension; heart failure such as (acute and chronic) congestive heart failure; left ventricular dysfunction; cardiac hypertrophy; cardiac fibrosis; cardiomyopathy (e.g., diabetic cardiac myopathy and post-infarction cardiac myopathy); supraventricular and ventricular arrhythmias; atrial fibrillation; atrial flutter; detrimental vascular remodeling; myocardial infarction and its sequelae; atherosclerosis; angina (whether unstable or stable); renal failure conditions, such as diabetic nephropathy; glomerulonephritis; renal fibrosis; scleroderma; glomerular sclerosis; microvascular complications, for example, diabetic retinopathy; renal vascular hypertension; vasculopathy; neuropathy; complications resulting from diabetes, including nephropathy, vasculopathy, retinopathy and neuropathy, diseases of the coronary vessels; proteinuria; albumenuria; post-surgical hypertension; metabolic syndrome; obesity; restenosis following angioplasty; eye diseases and associated abnormalities including raised intra-ocular pressure; glaucoma; retinopathy; abnormal vascular growth and remodeling; angiogenesis-related disorders, such as neovascular age related macular degeneration; hyperaldosteronism; anxiety states; and cognitive disorders (Fisher N. D.; Hollenberg N. K. Expert Opin. Investig. Drugs. 2001, 10, 417-26).

Elevated levels of βamyloid, the product of the activity of the well-characterized aspartic protease β-secretase (BACE) activity on amyloid precursor protein, are widely believed to be responsible for the development and progression of amyloid plaques in the brains of Alzheimer's disease patients. The secreted aspartic proteases of Candida albicans are associated with its pathogenic virulence (Naglik, J. R.; Challacombe, S. J.; Hube, B. Microbiology and Molecular Biology Reviews 2003, 67, 400-428). The viruses HIV and HTLV depend on their respective aspartic proteases for viral maturation. Plasmodium falciparum uses plasmepsins I and II to degrade hemoglobin.

A pharmaceutical composition of the invention may, alternatively or in addition to a compound of Formula I, comprise a pharmaceutically acceptable salt of a compound of Formula I or a prodrug or pharmaceutically active metabolite of such a compound or salt and one or more pharmaceutically acceptable carriers therefor.

The compositions of the invention are aspartic protease inhibitors. Said compositions contain compounds having a mean inhibition constant (IC₅₀) against aspartic proteases of between about 5,000 nM to about 0.01 nM; preferably between about 50 nM to about 0.01 nM; and more preferably between about 5 nM to about 0.01 nM.

The compositions of the invention reduce blood pressure. Said compositions include compounds having an IC₅₀ for renin of between about 5,000 nM to about 0.01 nM; preferably between about 50 nM to about 0.01 nM; and more preferably between about 5 nM to about 0.01 nM.

The invention includes a therapeutic method for treating or ameliorating an aspartic protease mediated disorder in a subject in need thereof comprising administering to a subject in need thereof an effective amount of a compound of Formula I, or the enantiomers, diastereomers, or salts thereof or composition thereof.

Administration methods include administering an effective amount (i.e., a therapeutically effective amount) of a compound or composition of the invention at different times during the course of therapy or concurrently in a combination form. The methods of the invention include all known therapeutic treatment regimens.

“Prodrug” means a pharmaceutically acceptable form of an effective derivative of a compound (or a salt thereof) of the invention, wherein the prodrug may be: 1) a relatively active precursor which converts in vivo to a compound of the invention; 2) a relatively inactive precursor which converts in vivo to a compound of the invention; or 3) a relatively less active component of the compound that contributes to therapeutic activity after becoming available in vivo (i.e., as a metabolite). See “Design of Prodrugs”, ed. H. Bundgaard, Elsevier, 1985.

“Metabolite” means a pharmaceutically acceptable form of a metabolic derivative of a compound (or a salt thereof) of the invention, wherein the derivative is an active compound that contributes to therapeutic activity after becoming available in vivo.

“Effective amount” means that amount of active compound agent that elicits the desired biological response in a subject. Such response includes alleviation of the symptoms of the disease or disorder being treated. The effective amount of a compound of the invention in such a therapeutic method is from about 10 mg/kg/day to about 0.01 mg/kg/day, preferably from about 0.5 mg/kg/day to 5 mg/kg/day.

The invention includes the use of a compound of the invention for the preparation of a composition for treating or ameliorating an aspartic protease mediated chronic disorder or disease or infection in a subject in need thereof, wherein the composition comprises a mixture one or more compounds of the invention and an optional pharmaceutically acceptable carrier.

“Pharmaceutically acceptable carrier” means compounds and compositions that are of sufficient purity and quality for use in the formulation of a composition of the invention and that, when appropriately administered to an animal or human, do not produce an adverse reaction.

“Aspartic protease mediated disorder or disease” includes disorders or diseases associated with the elevated expression or overexpression of aspartic proteases and conditions that accompany such diseases.

An embodiment of the invention includes administering a renin inhibiting compound of Formula I or composition thereof in a combination therapy (U.S. Pat. No. 5,821,232, U.S. Pat. No. 6,716,875, U.S. Pat. No. 5,663,188, Fossa, A. A.; DePasquale, M. J.; Ringer, L. J.; Winslow, R. L. “Synergistic effect on reduction in blood pressure with coadministration of a renin inhibitor or an angiotensin-converting enzyme inhibitor with an angiotensin II receptor antagonist” Drug Development Research 1994, 33(4), 422-8) with one or more additional agents for the treatment of hypertension including α-blockers, β-blockers, calcium channel blockers, diuretics, natriuretics, saluretics, centrally acting antiphypertensives, angiotensin converting enzyme (ACE) inhibitors, dual ACE and neutral endopeptidase (NEP) inhibitors, angiotensin-receptor blockers (ARBs), aldosterone synthase inhibitor, aldosterone-receptor antagonists, or endothelin receptor antagonist.

α-Blockers include doxazosin, prazosin, tamsulosin, and terazosin.

β-Blockers for combination therapy are selected from atenolol, bisoprol, metoprolol, acetutolol, esmolol, celiprolol, taliprolol, acebutolol, oxprenolol, pindolol, propanolol, bupranolol, penbutolol, mepindolol, carteolol, nadolol, carvedilol, and their pharmaceutically acceptable salts.

Calcium channel blockers include dihydropyridines (DHPs) and non-DHPs. The preferred DHPs are selected from the group consisting of amlodipine, felodipine, ryosidine, isradipine, lacidipine, nicardipine, nifedipine, nigulpidine, niludipine, nimodiphine, nisoldipine, nitrendipine, and nivaldipine and their pharmaceutically acceptable salts. Non-DHPs are selected from flunarizine, prenylamine, diltiazem, fendiline, gallopamil, mibefradil, anipamil, tiapamil, and verampimil and their pharmaceutically acceptable salts.

A diuretic is, for example, a thiazide derivative selected from amiloride, chlorothiazide, hydrochlorothiazide, methylchlorothiazide, and chlorothalidon.

Centrally acting antiphypertensives include clonidine, guanabenz, guanfacine and methyldopa.

ACE inhibitors include alacepril, benazepril, benazaprilat, captopril, ceronapril, cilazapril, delapril, enalapril, enalaprilat, fosinopril, lisinopril, moexipiril, moveltopril, perindopril, quinapril, quinaprilat, ramipril, ramiprilat, spirapril, temocapril, trandolapril, and zofenopril. Preferred ACE inhibitors are benazepril, enalpril, lisinopril, and ramipril.

Dual ACE/NEP inhibitors are, for example, omapatrilat, fasidotril, and fasidotrilat.

Preferred ARBs include candesartan, eprosartan, irbesartan, losartan, olmesartan, tasosartan, telmisartan, and valsartan.

Preferred aldosterone synthase inhibitors are anastrozole, fadrozole, and exemestane.

Preferred aldosterone-receptor antagonists are spironolactone and eplerenone.

A preferred endothelin antagonist is, for example, bosentan, enrasentan, atrasentan, darusentan, sitaxentan, and tezosentan and their pharmaceutically acceptable salts.

An embodiment of the invention includes administering an HIV protease inhibiting compound of Formula I or composition thereof in a combination therapy with one or more additional agents for the treatment of AIDS reverse transcriptase inhibitors, non-nucleoside reverse transcriptase inhibitors, other HIV protease inhibitors, HIV integrase inhibitors, entry inhibitors (including attachment, co-receptor and fusion inhibitors), antisense drugs, and immune stimulators.

Preferred reverse transcriptase inhibitors are zidovudine, didanosine, zalcitabine, stavudine, lamivudine, abacavir, tenofovir, and emtricitabine.

Preferred non-nucleoside reverse transcriptase inhibitors are nevirapine, delaviridine, and efavirenz.

Preferred HIV protease inhibitors are saquinavir, ritonavir, indinavir, nelfinavir, amprenavir, lopinavir, atazanavir, and fosamprenavir.

Preferred HIV integrase inhibitors are L-870,810 and S-1360.

Entry inhibitors include compounds that bind to the CD4 receptor, the CCR5 receptor or the CXCR4 receptor. Specific examples of entry inhibitors include enfuvirtide (a peptidomimetic of the HR2 domain in gp41) and sifurvitide.

A preferred attachment and fusion inhibitor is enfuvirtide.

An embodiment of the invention includes administering β-secretase inhibiting compound of Formula I or composition thereof in a combination therapy with one or more additional agents for the treatment of Alzheimer's disease including tacrine, donepezil, rivastigmine, galantamine, and memantine.

An embodiment of the invention includes administering a plasmepsin inhibiting compound of Formula I or composition thereof in a combination therapy with one or more additional agents for the treatment of malaria including artemisinin, chloroquine, halofantrine, hydroxychloroquine, mefloquine, primaquine, pyrimethamine, quinine, sulfadoxine.

Combination therapy includes co-administration of the compound of the invention and said other agent, sequential administration of the compound and the other agent, administration of a composition containing the compound and the other agent, or simultaneous administration of separate compositions containing of the compound and the other agent.

The invention further includes the process for making the composition comprising mixing one or more of the present compounds and an optional pharmaceutically acceptable carrier; and includes those compositions resulting from such a process, which process includes conventional pharmaceutical techniques.

The compositions of the invention include ocular, oral, nasal, transdermal, topical with or without occlusion, intravenous (both bolus and infusion), and injection (intraperitoneally, subcutaneously, intramuscularly, intratumorally, or parenterally). The composition may be in a dosage unit such as a tablet, pill, capsule, powder, granule, liposome, ion exchange resin, sterile ocular solution, or ocular delivery device (such as a contact lens and the like facilitating immediate release, timed release, or sustained release), parenteral solution or suspension, metered aerosol or liquid spray, drop, ampoule, auto-injector device, or suppository; for administration ocularly, orally, intranasally, sublingually, parenterally, or rectally, or by inhalation or insufflation.

Compositions of the invention suitable for oral administration include solid forms such as pills, tablets, caplets, capsules (each including immediate release, timed release, and sustained release formulations), granules and powders; and, liquid forms such as solutions, syrups, elixirs, emulsions, and suspensions. Forms useful for ocular administration include sterile solutions or ocular delivery devices. Forms useful for parenteral administration include sterile solutions, emulsions, and suspensions.

The compositions of the invention may be administered in a form suitable for once-weekly or once-monthly administration. For example, an insoluble salt of the active compound may be adapted to provide a depot preparation for intramuscular injection (e.g., a decanoate salt) or to provide a solution for ophthalmic administration.

The dosage form containing the composition of the invention contains a therapeutically effective amount of the active ingredient necessary to provide a therapeutic effect. The composition may contain from about 5,000 mg to about 0.5 mg (preferably, from about 1,000 mg to about 0.5 mg) of a compound of the invention or salt form thereof and may be constituted into any form suitable for the selected mode of administration. The composition may be administered about 1 to about 5 times per day. Daily administration or post-periodic dosing may be employed.

For oral administration, the composition is preferably in the form of a tablet or capsule containing, e.g., 500 to 0.5 milligrams of the active compound. Dosages will vary depending on factors associated with the particular patient being treated (e.g., age, weight, diet, and time of administration), the severity of the condition being treated, the compound being employed, the mode of administration, and the strength of the preparation.

The oral composition is preferably formulated as a homogeneous composition, wherein the active ingredient is dispersed evenly throughout the mixture, which may be readily subdivided into dosage units containing equal amounts of a compound of the invention. Preferably, the compositions are prepared by mixing a compound of the invention (or pharmaceutically acceptable salt thereof) with one or more optionally present pharmaceutical carriers (such as a starch, sugar, diluent, granulating agent, lubricant, glidant, binding agent, and disintegrating agent), one or more optionally present inert pharmaceutical excipients (such as water, glycols, oils, alcohols, flavoring agents, preservatives, coloring agents, and syrup), one or more optionally present conventional tableting ingredients (such as corn starch, lactose, sucrose, sorbitol, talc, stearic acid, magnesium stearate, dicalcium phosphate, and any of a variety of gums), and an optional diluent (such as water).

Binder agents include starch, gelatin, natural sugars (e.g., glucose and beta-lactose), corn sweeteners and natural and synthetic gums (e.g., acacia and tragacanth). Disintegrating agents include starch, methyl cellulose, agar, and bentonite.

Tablets and capsules represent an advantageous oral dosage unit form. Tablets may be sugarcoated or filmcoated using standard techniques. Tablets may also be coated or otherwise compounded to provide a prolonged, control-release therapeutic effect. The dosage form may comprise an inner dosage and an outer dosage component, wherein the outer component is in the form of an envelope over the inner component. The two components may further be separated by a layer which resists disintegration in the stomach (such as an enteric layer) and permits the inner component to pass intact into the duodenum or a layer which delays or sustains release. A variety of enteric and non-enteric layer or coating materials (such as polymeric acids, shellacs, acetyl alcohol, and cellulose acetate or combinations thereof) may be used.

Compounds of the invention may also be administered via a slow release composition; wherein the composition includes a compound of the invention and a biodegradable slow release carrier (e.g., a polymeric carrier) or a pharmaceutically acceptable non-biodegradable slow release carrier (e.g., an ion exchange carrier).

Biodegradable and non-biodegradable slow release carriers are well known in the art. Biodegradable carriers are used to form particles or matrices which retain an active agent(s) and which slowly degrade/dissolve in a suitable environment (e.g., aqueous, acidic, basic and the like) to release the agent. Such particles degrade/dissolve in body fluids to release the active compound(s) therein. The particles are preferably nanoparticles (e.g., in the range of about 1 to 500 nm in diameter, preferably about 50-200 nm in diameter, and most preferably about 100 nm in diameter). In a process for preparing a slow release composition, a slow release carrier and a compound of the invention are first dissolved or dispersed in an organic solvent. The resulting mixture is added into an aqueous solution containing an optional surface-active agent(s) to produce an emulsion. The organic solvent is then evaporated from the emulsion to provide a colloidal suspension of particles containing the slow release carrier and the compound of the invention.

The compound of Formula I may be incorporated for administration orally or by injection in a liquid form such as aqueous solutions, suitably flavored syrups, aqueous or oil suspensions, flavored emulsions with edible oils such as cottonseed oil, sesame oil, coconut oil or peanut oil and the like, or in elixirs or similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone, and gelatin. The liquid forms in suitably flavored suspending or dispersing agents may also include synthetic and natural gums. For parenteral administration, sterile suspensions and solutions are desired. Isotonic preparations, which generally contain suitable preservatives, are employed when intravenous administration is desired.

The compounds may be administered parenterally via injection. A parenteral formulation may consist of the active ingredient dissolved in or mixed with an appropriate inert liquid carrier. Acceptable liquid carriers usually comprise aqueous solvents and other optional ingredients for aiding solubility or preservation. Such aqueous solvents include sterile water, Ringer's solution, or an isotonic aqueous saline solution. Other optional ingredients include vegetable oils (such as peanut oil, cottonseed oil, and sesame oil), and organic solvents (such as solketal, glycerol, and formyl). A sterile, non-volatile oil may be employed as a solvent or suspending agent. The parenteral formulation is prepared by dissolving or suspending the active ingredient in the liquid carrier whereby the final dosage unit contains from 0.005 to 10% by weight of the active ingredient. Other additives include preservatives, isotonizers, solubilizers, stabilizers, and pain-soothing agents.

Injectable suspensions may also be prepared, in which case appropriate liquid carriers, suspending agents and the like may be employed.

Compounds of the invention may be administered intranasally using a suitable intranasal vehicle.

Compounds of the invention may also be administered topically using a suitable topical transdermal vehicle or a transdermal patch.

For ocular administration, the composition is preferably in the form of an ophthalmic composition. The ophthalmic compositions are preferably formulated as eye-drop formulations and filled in appropriate containers to facilitate administration to the eye, for example a dropper fitted with a suitable pipette. Preferably, the compositions are sterile and aqueous based, using purified water. In addition to the compound of the invention, an ophthalmic composition may contain one or more of: a) a surfactant such as a polyoxyethylene fatty acid ester; b) a thickening agents such as cellulose, cellulose derivatives, carboxyvinyl polymers, polyvinyl polymers, and polyvinylpyrrolidones, typically at a concentration in the range of about 0.05 to about 5.0% (wt/vol); c) (as an alternative to or in addition to storing the composition in a container containing nitrogen and optionally including a free oxygen absorber such as Fe), an anti-oxidant such as butylated hydroxyanisol, ascorbic acid, sodium thiosulfate, or butylated hydroxytoluene at a concentration of about 0.00005 to about 0.1% (wt/vol); d) ethanol at a concentration of about 0.01 to 0.5% (wt/vol); and e) other excipients such as an isotonic agent, buffer, preservative, and/or pH-controlling agent. The pH of the ophthalmic composition is desirably within the range of 4 to 8.

### Methods of Preparation

In the discussion below R¹, R², Y, A, Q, R⁴, L, R⁵, R^{5a}, R⁶, R^{6a}, L, G, R^{e}, and R^{f} are defined as described above for compounds of Formula I. In cases where the synthetic intermediates and final products of Formula I described below contain potentially reactive functional groups, for example amino, hydroxyl, thiol and carboxylic acid groups, that may interfere with the desired reaction, it may be advantageous to employ protected forms of the intermediate. Methods for the selection, introduction and subsequent removal of protecting groups are well known to those skilled in the art. (T. W. Greene and P. G. M. Wuts “Protective Groups in Organic Synthesis” John Wiley & Sons, Inc., New York 1999). Such protecting group manipulations are assumed in the discussion below and not usually described explicitly. Generally, reagents in the reaction schemes are used in equimolar amounts; however, in certain cases it may be desirable to use an excess of one reagent to drive a reaction to completion. This is especially the case when the excess reagent can be readily removed by evaporation or extraction. Bases employed to neutralize HCl in reaction mixtures are generally used in slight to substantial excess (1.05-5 equivalents).

In the first process, a compound of Formula I is prepared by reaction of an intermediate of Formula II with an amine intermediate of Formula III:

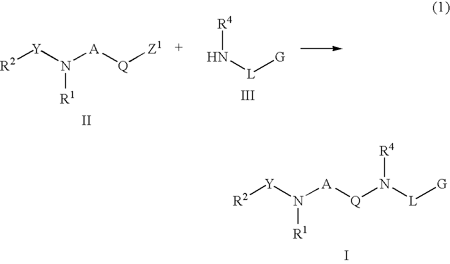

wherein Z¹ in II is a leaving group such as halide, alkanesulfonate, haloalkanesulfonate, arylsulfonate, aryloxide, heteroaryloxide, azole, azolium salt, or alkoxide.

In a second process, a compound of Formula I is prepared by reaction of a compound of Formula IV with a compound of Formula V wherein Z¹ is a leaving group such as halide, alkanesulfonate, arylsulfonate, aryloxide, azole, azolium salt, or alkoxide:

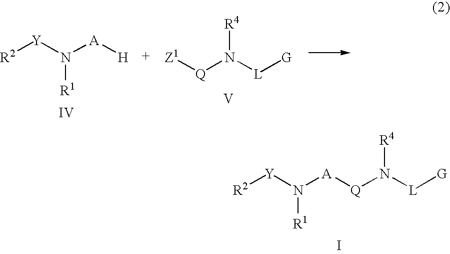

wherein the H atom in IV is attached to a nitrogen atom that is part of A.

In a third process, a compound of Formula I wherein G is NHR^{e} or NR^{e}R^{f} is prepared by reductive amination of an aldehyde of Formula VI wherein L* is a linear (C₁-C₃)alkyl chain with an amine of Formula R^{e}NH₂ or R^{e}R^{f}NH using a reducing agent such as NaCNBH₃ or NaBH(OAc)₃:

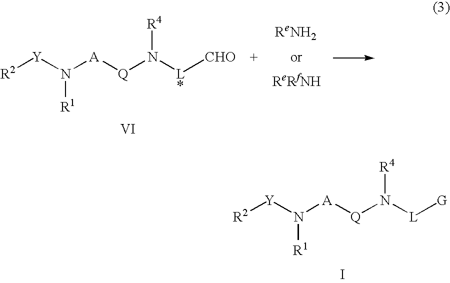

In a fourth process, a compound of Formula I wherein G is NH₂ is prepared by reduction of an azide of Formula VII using catalytic hydrogenation or Ph₃P in wet THF:

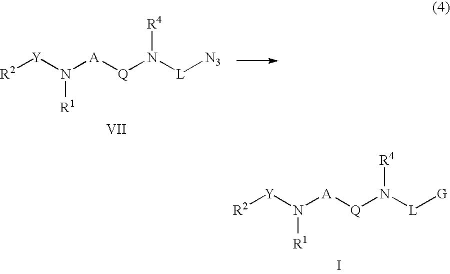

In a fifth process, a compound of Formula I wherein R¹ is phenyl, heteroaryl or bicyclic heteroaryl is prepared by reaction of a compound of Formula VIII with a halide of Formula IX wherein Z² is chlorine, bromine or iodine in the presence of a palladium or copper catalyst:

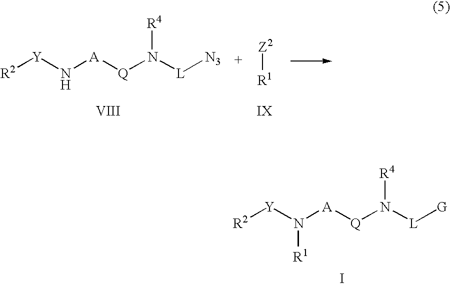

Alternatively Z² in intermediate IX is B(OH)₂ and a copper catalyst is used.

In a sixth process, a compound of Formula I wherein A is a saturated or partially unsaturated ring is prepared by reaction of an amine intermediate of Formula X with a ketone of Formula XI using a reducing agent such as NaCNBH₃ or NaBH(OAc)₃:

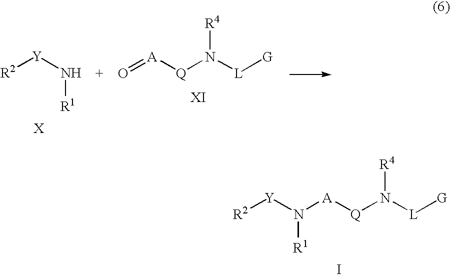

In a seventh process, compounds of Formula I can also be prepared from other compounds of Formula I and protected compounds of Formula I:

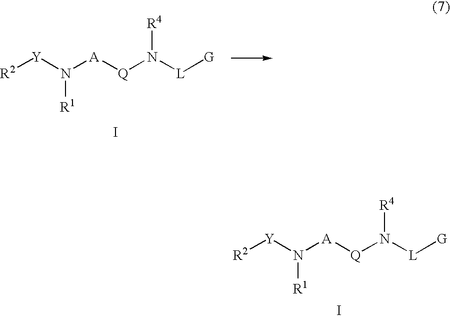

For example:
(1) when R¹ is bromophenyl or iodophenyl it may be transformed into a compound in which R¹ is biphenyl by palladium catalyzed coupling with a phenylboronic acid under Suzuki conditions;
(2) when R¹ is bromophenyl or iodophenyl it may be transformed a compound in which R¹ is alkynylphenyl by palladium catalyzed coupling with a terminal alkyne under Sonogashira conditions;
(3) when R¹ is bromophenyl or iodophenyl it may be transformed a compound in which R¹ is allylphenyl by palladium catalyzed coupling with tetraallyltin using a Stille conditions;
(4) when R¹ is bromophenyl or iodophenyl it may be transformed a compound in which R¹ is cyanophenyl using CuCN;
(5) when R¹ is hydroxyphenyl it may be alkylated with an alkyl halide, cycloalkyl halide or cycloalkylalkyl halide in the presence of a base such as sodium hydride to give a compound in which R¹ is alkoxyphenyl, cycloalkoxyphenyl or cycloalkylalkoxyphenyl;
(6) when Q is Q1 it may be transformed into a compound in which Q is Q2 by treatment with P₂S₅ or Lawesson's reagent;
(7) when R² is alkenyloxy it may be transformed into a compound in which R² is hydroxyalkyl by hydroboration;
(8) when R² is hydroxyalkyl substituted it may be transformed into a compound in which R² is R^{2a}—O—NH—C(O)—Y₁— by the following steps: conversion of the hydroxyl to the corresponding methanesulfonate, displacement of the methanesulfonate by azide anion, reduction of the azide and acylation with R^{2a}—O—C(O)—Cl.

Intermediates of Formula II wherein Z¹=chlorine and Q is Q1 that is attached to a carbon atom that is part of A are prepared from intermediates XII:

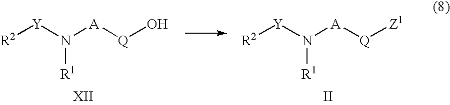

by reaction with, for example, thionyl chloride or oxalyl chloride.

Intermediates of Formula II wherein Q is Q5, Q is attached to a nitrogen atom that is part of A and Z′ is methoxy are prepared from intermediates XIII by reaction with 3,4-dimethoxy-3-cyclobutene-1,2-dione:

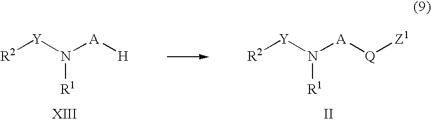

Intermediates of Formula II wherein Q is Q1, Q1 is attached to a nitrogen atom that is part of A and Z¹ is chlorine, 1-imidazolyl, or p-nitrophenoxy are prepared from intermediates of formula XIII wherein H is attached to a nitrogen atom that is part of A by reaction with phosgene, 1,1′-carbonyldiimidazole, or p-nitrophenyl chloroformate respectively.

Intermediates of Formula XIII wherein A is a saturated or partially unsaturated ring are prepared by reductive alkylation of amine intermediates of formula XIV with ketone intermediates of Formula XV:

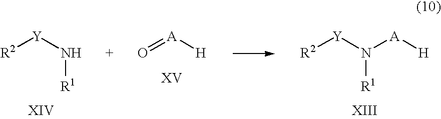

Intermediates of Formula XIII wherein A is a saturated or partially unsaturated ring are prepared by reaction of amine intermediates of Formula XVI with a compound of Formula XVII:

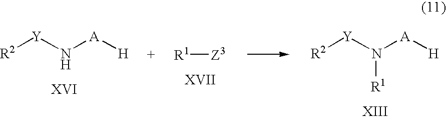

When R¹ is a cycloalkyl group, Z³ is a halide, preferably bromide or iodide, or an alkyl, haloalkylsulfonate or arylsulfonate and a base such as i-Pr₂NEt or K₂CO₃ is used. When R¹ is a phenyl, heteroaryl or bicyclic heteroaryl, Z³ is a halide, preferably bromide or iodide, or trifluoromethanesulfonate and a palladium or copper catalyst is employed. Alternatively, when R¹ is a phenyl, heteroaryl or bicyclic heteroaryl, Z³ is —B(OH)₂ and a copper catalyst is used. (Ley, S. V.; Thomas, A. W. Angew. Chem. Int. Ed. 2003, 42, 5400-5449).

Intermediates of Formula XIII are prepared by alkylation of amine intermediates of Formula XVIII with halides of Formula XIX wherein Z⁴ is a halide or sulfonate leaving group or by reductive alkylation with aldehydes of Formula XX wherein Y* is (C₁-C₉)alkylene:

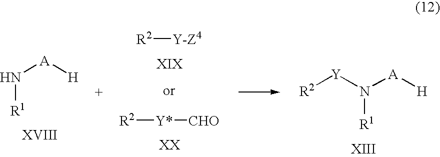

Intermediates of Formula XIII are prepared by acylation of amine intermediates of Formula XVIII with carboxylic acid derivatives of Formula XXI, followed by reduction of the resulting amide XXII:

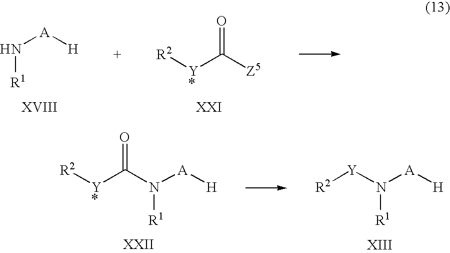

Amine intermediates of Formulae XVI and XVIII are prepared by reductive amination of ketone intermediates of Formula XV with amines of Formulae XXIII and XXIV respectively:

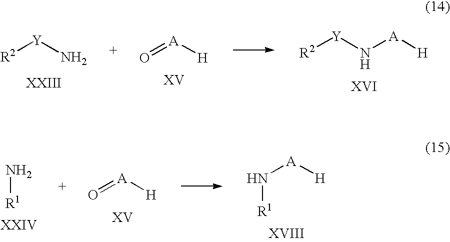

Intermediates of Formulae XVI and XVIII are prepared by alkylation or arylation of amine intermediates of Formula XXV with intermediates of Formulae XIX (or XX) or XVII respectively:

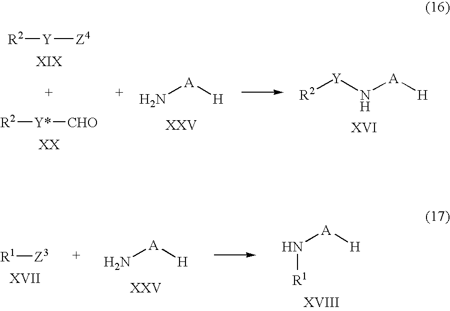

Intermediates of Formula XII wherein A is an aromatic or heteroaromatic ring are prepared from amine intermediates of Formula XIV and halide intermediates of Formula XXVI wherein Z⁶ is chloride or, preferably bromide or iodide using a palladium or copper catalyst:

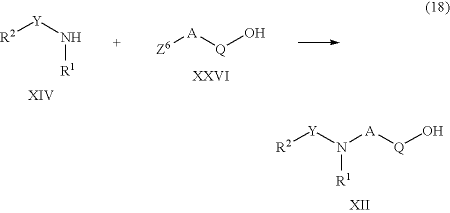

Intermediates of Formula XII are also prepared by procedures analogous to those outlined in Equations 10-17. Thus sequences including N-arylation, N-alkylation and reductive alkylation of amines of Formula XXVII affords intermediates of Formula XII:

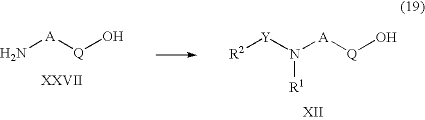

Intermediates of Formula XXVII are prepared by Curtius rearrangement of carboxylic acid intermediates of Formula XXVIII:

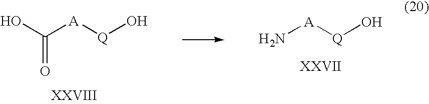

Similarly intermediates of Formula XXV are prepared by Curtius rearrangement of carboxylic acid intermediates of Formula XXIX:

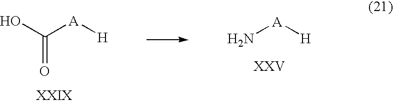

Intermediates of Formula III wherein L is a C₂ alkyl chain are prepared from natural and unnatural α-amino acids and by other methods (Lucet, D.; Le Gall, T.; Mioskowski, C. Angew. Chem. Int. Ed. 1998, 37, 2580-2617). Likewise, intermediates of Formula V and VII wherein L is a C₃ or C₄ alkyl chain are prepared from β- and γ-amino acids, respectively.

The invention is further defined by reference to the examples, which are intended to be illustrative and not limiting.

Representative compounds of the invention can be synthesized in accordance with the general synthetic schemes described above and are illustrated in the examples that follow. The methods for preparing the various starting materials used in the schemes and examples are well within the knowledge of persons skilled in the art.

The following abbreviations have the indicated meanings:

| Abbreviation | Meaning |
|---|---|
| aq | aqueous |
| Boc | tert-butoxy carbonyl or t-butoxy carbonyl |
| (Boc)₂O | di-tert-butyl dicarbonate |
| brine | saturated aqueous NaCl |
| CH₂Cl₂ | methylene chloride |
| CH₃CN or MeCN | acetonitrile |
| Cpd | compound |
| d | day |
| DBU | 1,8-diazabicyclo[5.4.0]undec-7-ene |
| DIEA | N,N-diisopropylethylamine |
| DMAP | 4-(dimethylamino)pyridine |
| DMF | N,N-dimethylformamide |
| DMPU | 1,3-dimethyl-3,4,5,6-tetrahydro-2(1H)-pyrimidinone |
| EDC.HCl | 1-[3-(dimethylamino)propyl]-3-ethylcarbodiimide |
| | hydrochloride |
| equiv | equivalents |
| Et | ethyl |
| Et₂O | ethyl ether |
| EtOAc | ethyl acetate |
| Fmoc | 1-[[(9H-fluoren-9-ylmethoxy)carbonyl]oxy]- |
| Fmoc-OSu | 1-[[(9H-fluoren-9-ylmethoxy)carbonyl]oxy]-2,5- |
| | pyrrolidinedione |
| h, hr | hour |
| HOBt | 1-hydroxybenzotriazole |
| HATU | 2-(7-Aza-1H-benzotriazole-1-yl)-1,1,3,3- |
| | tetramethyluronium hexafluorophosphate |
| HBTU | 2-(1H-Benzotriazole-1-yl)-1,1,3,3-tetramethyluronium |
| | hexafluorophosphate |
| KHMDS | potassium hexamethyldisilazane |
| LAH or LiAlH₄ | lithium aluminum hydride |
| LC-MS | liquid chromatography-mass spectroscopy |
| LHMDS | lithium hexamethyldisilazane |
| Me | methyl |
| MeCN | acetonitrile |
| MeOH | methanol |
| MsCl | methanesulfonyl chloride |
| min | minute |
| MS | mass spectrum |
| NaH | sodium hydride |
| NaHCO₃ | sodium bicarbonate |
| NaN₃ | sodium azide |
| NaOH | sodium hydroxide |
| Na₂SO₄ | sodium sulfate |
| NMP | N-methylpyrrolidinone |
| Pd₂(dba)₃ | tris(dibenzylideneacetone)dipalladium(0) |
| Ph | phenyl |
| rt | room temperature |
| satd | saturated |
| SOCl₂ | thionyl chloride |
| TBAF | tetrabutylammonium fluoride |
| TEA | triethylamine or Et₃N |
| TEAF | tetraethylammonium fluoride |
| TEMPO | 2,2,6,6-tetramethyl-1-piperidinyloxy, free radical |
| Teoc | 1-[2-(trimethylsilyl)ethoxycarbonyloxy]- |
| Teoc-OSu | 1-[2-(trimethylsilyl)ethoxycarbonyloxy]pyrrolidin-2,5- |
| | dione |
| TFA | trifluoroacetic acid |
| THF | tetrahydrofuran |
| TMSCl | chlorotrimethylsilane or trimethylsilyl chloride |
| t_{R} | retention time |

### Purification Methods

Preparative HPLC refers to reverse phase HPLC on a C-18 column eluted with a water/acetonitrile gradient containing 0.01% TFA run on a Gilson 215 system.

Chromatography on silica gel refers to normal phase chromatography on a silica gel column or cartridge eluted with an hexanes/EtOAc gradient.

Preparative TLC refers to normal phase thin or thick layer chromatography on a silica gel plate eluted with an organic solvents or mixtures of organic solvents, such as hexanes/EtOAc mixtures.

Chiral HPLC refers to normal phase chromatography on a chiral column, such as chiralcel OD-H or AD-H, eluted with a mixture of organic solvents such as isopropanol in hexanes buffered with diethylamine

### Analytical Methods

LC-MS (3 min)

Column: Chromolith SpeedRod, RP-18e, 50×4.6 mm; Mobil phase: A: 0.01% TFA/water, B: 0.01% TFA/CH₃CN; Flow rate: 1 mL/min; Gradient:

| Time (min) | A % | B % |
|---|---|---|
| 0.0 | 90 | 10 |
| 2.0 | 10 | 90 |
| 2.4 | 10 | 90 |
| 2.5 | 90 | 10 |
| 3.0 | 90 | 10 |

### Electrospray Ionization

### Preparation A

### Methyl 3-((3-chlorophenyl)(piperidin-3-yl)amino)propylcarbamate

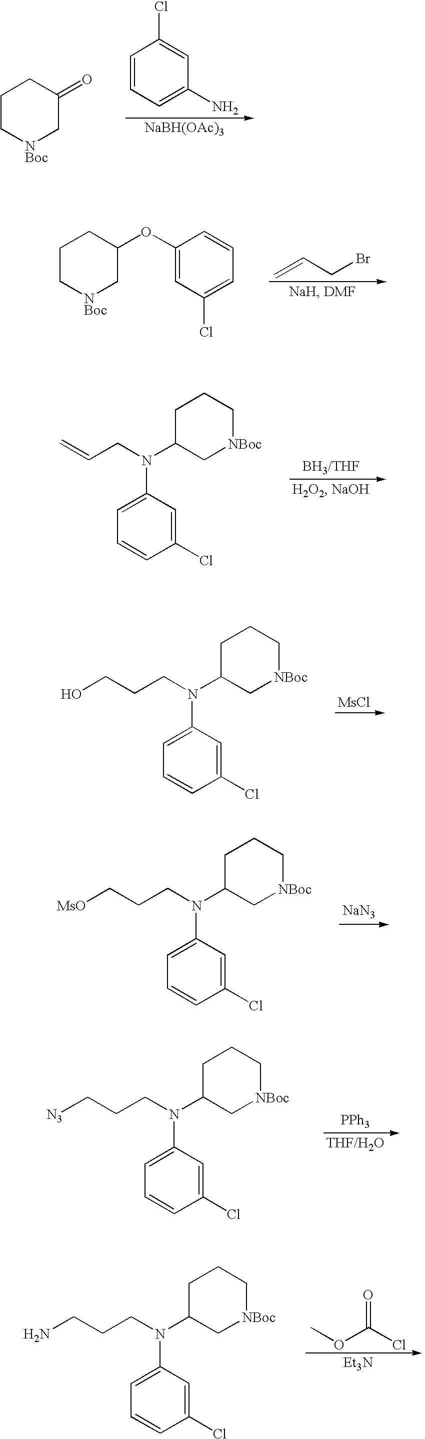

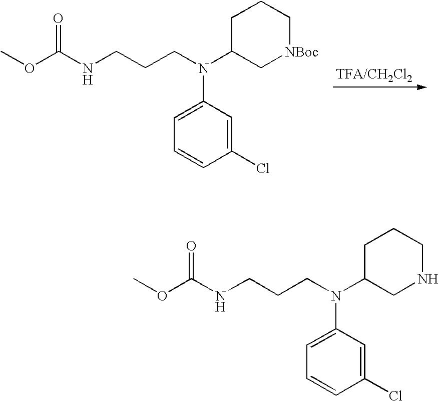

### Step 1. tert-butyl 3-(3-chlorophenylamino)piperidine-1-carboxylate

To a solution of tert-butyl 3-oxopiperidine-1-carboxylate (4.2 g, 21 mmol) in CH₂Cl₂ (60 mL) was added 3-chlorobenzenamine (2.7 g, 21 mmol). The reaction mixture was stirred at room temperature for 30 min and then cooled to 0° C., NaBH (OAc)₃ (9.0 g, 42 mmol) was added. The resulting mixture was allowed to warm to room temperature and stirred for 18 h. The reaction mixture was diluted with CH₂Cl₂, washed with saturated sodium bicarbonate and brine, dried over Na₂SO₄, and concentrated to the crude product which was purified by column chromatography on silica gel to afford tert-butyl 3-(3-chlorophenylamino)piperidine-1-carboxylate (2.5 g, 38%). ¹H NMR (CDCl₃, 400 MHz) δ 1.44 (s, 9H), 1.55 (m, 2H), 1.72 (m, 1H), 2.03 (m, 1H), 2.94 (m, 1H), 3.10 (m, 1H), 2.35 (m, 1H), 3.66 (d, 1H), 3.95 (d, 1H), 6.54 (d, 1H), 6.64 (s, 1H), 6.68 (d, 1H), 7.08 (t, 1H).

### Step 2. tert-butyl 3-(allyl(3-chlorophenyl)amino)piperidine-1-carboxylate

NaH (960 mg, 24 mmol) was slowly added to a solution of allyl bromide (4.84 g, 40 mmol) and tert-butyl 3-(3-chlorophenylamino)piperidine-1-carboxylate (2.5 g, 8 mmol) in dry DMF (10 mL) under nitrogen atmosphere at 0° C. in a sealed tube. The reaction mixture was then heated to 100° C. After 17 h, the solution was cooled to 0° C., quenched with a saturated aqueous NH₄Cl (90 mL), and extracted with Et₂O (3×100 mL). The combined organic layers were dried over Na₂SO₄ and the solvent evaporated. The crude product was purified by column chromatography on silica gel to afford tert-butyl 3-(allyl(3-chlorophenyl)amino)piperidine-1-carboxylate (800 mg, 29%). ¹H NMR (CDCl₃, 400 MHz) δ 1.45 (m, 2H), 1.63-1.92 (m, 5H), 2.85-3.08 (m, 4H), 3.87 (m, 3H), 5.18 (m, 4H), 5.85 (m, 2H), 6.62 (m, 1H), 6.71 (t, 1H), 7.08 (t, 1H).

### Step 3. tert-butyl 3-((3-chlorophenyl)(3-hydroxypropyl)amino)piperidine-1-carboxylate

To a solution of tert-butyl 3-(allyl(3-chlorophenyl)amino)piperidine-1-carboxylate (800 mg, 2.28 mmol) in dry THF (8 mL) was added dropwise 2 M BH₃.THF (1.14 mL, 2.28 mmol) at 0° C. After 2 h at room temperature, the reaction mixture was cooled to 0° C. prior to successive addition of water (260 μL), 3 M aqueous NaOH (800 μL) and 30% H₂O (560 μL). The mixture was stirred for 2-3 h at room temperature and diluted with water (23 mL). The pH was adjusted to 6-7 with 0.5 N HCl. The aqueous phase was extracted with EtOAc (3×50 mL) and the combined organic layers were washed with saturated aqueous NaHCO₃ solution (56 mL) and brine (56 mL), dried over Na₂SO₄, and concentrated in vacuo. The crude product was purified by preparative TLC to afford tert-butyl 3-((3-chlorophenyl)(3-hydroxypropyl)amino)piperidine-1-carboxylate (400 mg, 48%). ¹H NMR (CDCl₃, 400 MHz) δ 1.47 (s, 9H), 1.50-1.82 (m, 7H), 1.95 (m, 1H), 2.62 (m, 2H), 3.38-3.54 (m, 3H), 3.73 (t, 2H), 4.16 (m, 2H), 6.72 (m, 2H), 6.79 (m, 1H), 7.13 (t, 1H).

### Step 4. tert-butyl 3-((3-chlorophenyl)(3-(methylsulfonyloxy)propyl)amino)piperidine-1-carboxylate

To a solution of tert-butyl 3-((3-chlorophenyl)(3-hydroxypropyl)amino)piperidine-1-carboxylate (500 mg, 1.35 mmol) in dry CH₂Cl₂ (8 mL) was added Et₃N (410 mg, 4.06 mmol) at 0° C. Then a solution of MsCl (210 mg, 1.76 mmol) in dry CH₂Cl₂ (3 mL) was added dropwise. After addition, it was allowed to warm to room temperature gradually. Upon completion of the reaction, water (20 mL) was added and the aqueous layer was extracted with CH₂Cl₂ (3×20 mL). The combined organic layers were washed with 10% citric acid, sat. NaHCO₃ and brine, then dried over Na₂SO₄, filtered and concentrated to give tert-butyl 3-((3-chlorophenyl)(3-(methylsulfonyloxy)propyl)amino)piperidine-1-carboxylate (600 mg, 99%), which was used in the next step without purification.

### Step 5. tert-butyl 3-((3-azidopropyl)(3-chlorophenyl)amino)piperidine-1-carboxylate

tert-Butyl 3-((3-chlorophenyl)(3-(methylsulfonyloxy)propyl)amino)piperidine-1-carboxylate (600 mg, 1.35 mmol) was dissolved into anhydrous DMF (10 mL), solid NaN₃ (270 mg, 4.06 mmol) was added and the reaction mixture was heated to 70° C. The reaction mixture was cooled to room temperature and then was diluted with ethyl acetate (80 mL) and water (20 mL). The organic phase was washed with water (3×20 mL), dried over Na₂SO₄ and evaporated to give tert-butyl 3-((3-azidopropyl)(3-chlorophenyl)amino)piperidine-1-carboxylate (600 mg, 99%), which was used in the next step without purification.

### Step 6. tert-butyl 3-((3-aminopropyl)(3-chlorophenyl)amino)piperidine-1-carboxylate

To a solution of tert-butyl 3-((3-azidopropyl)(3-chlorophenyl)amino)piperidine-1-carboxylate (600 mg, 1.53 mmol) in THF/H₂O (20:1, 10.5 mL) was added PPh₃ (1.6 g, 6.1 mmol). The reaction mixture was stirred at room temperature overnight. The solvent was removed under reduced pressure to afford the crude product which was purified by column chromatography on silica gel to afford tert-butyl 3-((3-aminopropyl)(3-chlorophenyl)amino)piperidine-1-carboxylate (350 mg, 62%). ¹HNMR (CDCl₃, 400 MHz) δ 1.47 (s, 9H), 1.50-1.83 (m, 7H), 1.96 (m, 1H), 2.63 (m, 2H), 2.76 (t, 1H), 3.27 (m, 2H), 3.51 (m, 1H), 4.14 (m, 2H), 6.66 (m, 2H), 6.74 (m, 1H), 7.14 (t, 1H).

### Step 7. tert-butyl 3-((3-(methoxycarbonylamino)propyl)(3-chlorophenyl)amino)piperidine-1-carboxylate

To a solution of tert-butyl 3-((3-aminopropyl)(3-chlorophenyl)amino)piperidine-1-carboxylate (350 mg, 0.95 mmol) and DMAP (58 mg, 0.48 mmol) in dry CH₂Cl₂ (3 mL), Et₃N (288 mg, 2.85 mmol) was added. The resulting mixture was cooled to 0-5° C. under ice-water bath, a solution of methyl chloroformate (450 mg, 4.75 mmol) in dry CH₂Cl₂ (2 mL) was added drop wise. After addition, the reaction mixture was stirred for 1-2 h at 0-5° C. Upon completion of the reaction, water (5 mL) was added. The aqueous layer was extracted with CH₂Cl₂ (3×15 mL). The combined organic layers were washed with 10% citric acid (2×10 mL) and brine, then dried over Na₂SO₄, filtered and concentrated to afford tert-butyl 3-((3-(methoxycarbonylamino)propyl)(3-chlorophenyl)amino)piperidine-1-carboxylate)-(3-methoxycarbonylamino-propyl)-amino]-piperidine-1-carboxylic acid tert-butyl ester (400 mg, 98%), which was used in the next step without further purification.

### Step 8. methyl 3-((3-chlorophenyl)(piperidin-3-yl)amino)propylcarbamate

tert-Butyl 3-((3-(methoxycarbonylamino)propyl)(3-chlorophenyl)amino)piperidine-1-carboxylate (80 mg, 0.188 mmol) was dissolved in a solution of 20% (V/V) TFA/CH₂Cl₂ (2 mL). The reaction mixture was stirred at room temperature for 1 h, a solution of saturated sodium bicarbonate was added dropwise to adjust pH=7-8. The resulting mixture was extracted with CH₂Cl₂ (3×10 mL), washed with brine, dried over Na₂SO₄, concentrated in vacuo to afford methyl 3-((3-chlorophenyl)(piperidin-3-yl)amino)propylcarbamate (61 mg, 100%), which was used in the next step directly without further purification.

The following compounds were prepared following procedures analogous to those described above:
1) methyl 3-((5-chloro-2-methylphenyl)(piperidin-3-yl)amino)propylcarbamate using 5-chloro-2-methylaniline in Step 1.

### Preparation B

### tert-butyl (S)-2-amino-3-((R)-tetrahydro-2H-pyran-3-yl)propyl(methyl)carbamate

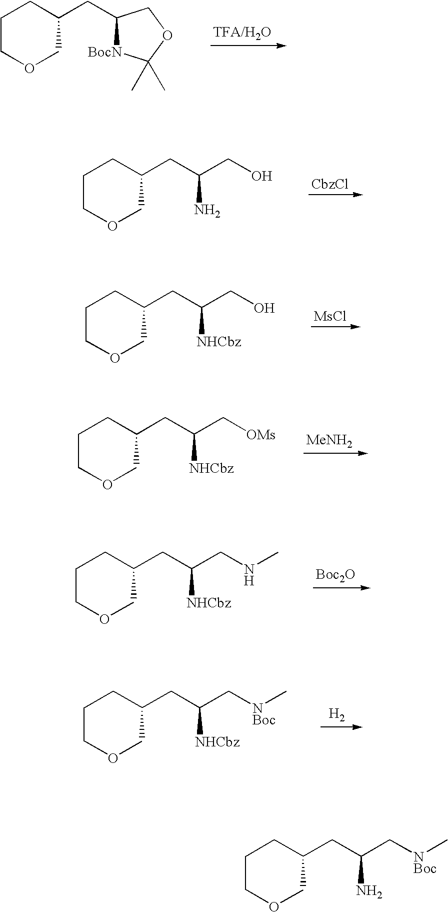

### Step 1. (S)-2-amino-3-((R)-tetrahydro-2H-pyran-3-yl)propan-1-ol

(S)-tert-butyl 2,2-dimethyl-4-(((R)-tetrahydro-2H-pyran-3-yl)methyl)oxazolidine-3-carboxylate was prepared using procedures described in U.S. Provisional App. No. 60/736,564 filed on Nov. 14, 2005 and PCT App No. PCT/US2006/043920 filed Nov. 13, 2006, the entire contents of which are hereby incorporated by reference.

(S)-tert-butyl 2,2-dimethyl-4-(((R)-tetrahydro-2H-pyran-3-yl)methyl)oxazolidine-3-carboxylate (32 g) was dissolved in a mixture of TFA (160 mL) and water (160 mL). The mixture was stirred at room temperature for 10 mins. The mixture was then concentrated in vacuo to give the crude product (50 g), which was used for the next step without further purification.

### Step 2. benzyl (S)-1-hydroxy-3-((R)-tetrahydro-2H-pyran-3-yl)propan-2-ylcarbamate

To a solution of (S)-2-amino-3-((R)-tetrahydro-2H-pyran-3-yl)propan-1-ol (50 g, 312.5 mmol) and K₂CO₃ (129.375 g, 937.5 mmol) in dioxane (250 mL) and water (250 mL) at 0° C. was added CbzCl (106.6 g, 625 mmol) dropwise. The reaction mixture was stirred at room temperature until the staring material disappeared. The organic solvent was distilled and the residue was dissolved in AcOEt (200 mL). The organic phase was separated and the water was extracted with AcOEt (3×100 ml). The combined organic phase was washed with water (3×50 ml), dried over Na₂SO₄, and concentrated in vacuo to give the residue, which was purified by column to give the product (26 g). ¹HNMR (CD₃OD) δ 1.1-1.3 (m, 2H), 1.4-1.5 (m, 1H), 1.5-1.7 (m, 3H), 1.8-1.9 (m, 1H), 2.6-2.8 (m, 1H), 3.0-3.1 (t, 1H), 3.3-3.4 (m, 1H), 3.5-3.6 (m, 1H), 3.6-3.8 (m, 2H), 3.8-3.9 (m, 2H), 5.0-5.1 (s, 2H), 7.3-7.4 (m, 5H).

### Step 3. (S)-2-(benzyloxycarbonylamino)-3-((R)-tetrahydro-2H-pyran-3-yl)propyl methanesulfonate

To a solution of benzyl (S)-1-hydroxy-3-((R)-tetrahydro-2H-pyran-3-yl)propan-2-ylcarbamate (26 g, 69.99 mmol) and triethylamine (21.21 g, 210 mmol) in CH₂Cl₂ at 0° C. was added mesyl chloride (16.1 g, 140 mmol) drop wise. The reaction mixture was stirred at room temperature until the staring material disappeared. The reaction was quenched with ice-cold water, extracted with CH₂Cl₂ (3×100 ml), washed with water (3×50 ml), dried over Na₂SO₄, and concentrated in vacuo to give the product (36 g), which was used for the next step without purification.

### Step 4. benzyl (S)-1-(methylamino)-3-((R)-tetrahydro-2H-pyran-3-yl)propan-2-ylcarbamate

To the ethanol solution of MeNH₂ (360 mL) was added (S)-2-(benzyloxycarbonylamino)-3-((R)-tetrahydro-2H-pyran-3-yl)propyl methanesulfonate (36 g, 106.7 mmol). The mixture was stirred at 30-40° C. overnight. Upon completion of the reaction, the solution was concentrated to give the product (40 g), which was used without further purification.

### Step 5. benzyl (S)-1-(N-methyl-tert-butoxycarbonylamino)-3-((R)-tetrahydro-2H-pyran-3-yl)propan-2-ylcarbamate

To the solution of benzyl (S)-1-(methylamino)-3-((R)-tetrahydro-2H-pyran-3-yl)propan-2-ylcarbamate (40 g, 130.5 mmol) and TEA (39.6 g, 391.5 mmol) in CH₂Cl₂ (400 mL) was added Boc₂O (56.9 g, 261 mmol). The mixture was stirred at room temperature until the starting material disappeared. The mixture was concentrated in vacuo to give the crude product, which was purified through column chromatography to give the product (20 g). ¹HNMR (CD₃OD) δ 1.2-1.4 (m, 3H), 1.4-1.5 (s, 9H), 1.5-1.6 (m, 2H), 1.6-1.7 (m, 1H), 1.8-1.9 (m, 1H), 2.8-2.9 (s, 3H), 2.9-3.1 (m, 2H), 3.3-3.4 (m, 1H), 3.5-3.6 (m, 12H), 3.7-3.9 (dd, 3H), 5.0-5.1 (s, 2H), 7.3-7.4 (m, 5H).

### Step 6. tert-butyl (S)-2-amino-3-((R)-tetrahydro-2H-pyran-3-yl)propyl(methyl)carbamate

To a solution of benzyl (S)-1-(N-methyl-tert-butoxycarbonylamino)-3-((R)-tetrahydro-2H-pyran-3-yl)propan-2-ylcarbamate (20 g, 49.2 mmol) in MeOH (400 mL) was added Pd(OH)₂ (2 g). The reaction bottle was degassed and filled into H₂. Upon completion of the reaction, the mixture was filtered through Celite, dried over Na₂SO₄, and concentrated to give the product (12 g). ¹HNMR (CD₃OD) δ 1.0-1.2 (m, 3H), 1.4-1.5 (m, 9H), 1.6-1.7 (m, 2H), 1.7-1.9 (m, 2H), 2.8-2.9 (s, 3H), 2.9-3.1 (m, 4H), 3.3-3.4 (m, 1H), 3.8-3.9 (m, 2H).

### Example 1

### methyl 3-((5-chloro-2-methylphenyl)((R)-1-((S)-1-cyclohexyl-3-(methylamino)propan-2-ylcarbamoyl)piperidin-3-yl)amino)propylcarbamate

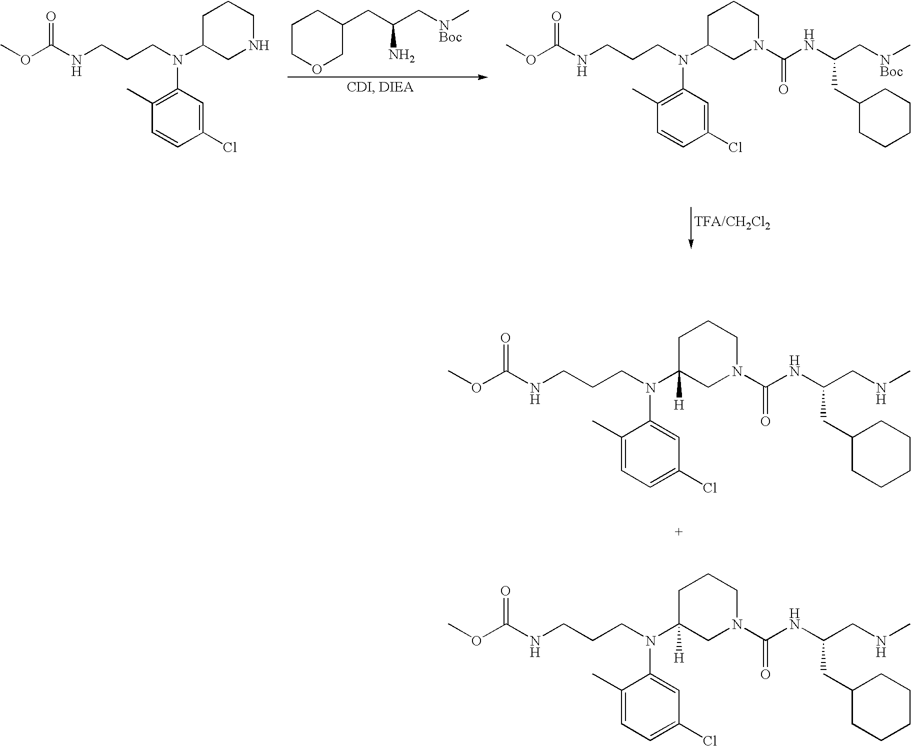

### Step 1. tert-butyl (S)-2-(3-((3-(methoxycarbonylamino)propyl)(5-chloro-2-methylphenyl)amino)piperidine-1-carboxamido)-3-cyclohexylpropyl(methyl)carbamate

(S)-tert-butyl 2-amino-3-cyclohexylpropyl(methyl)carbamate (48 mg, 0.177 mmol), prepared using procedures described in U.S. Provisional App. No. 60/616,770 filed on Oct. 7, 2004 and PCT App No. PCT/US2005/036230 filed Oct. 7, 2005 the entire contents of which are hereby incorporated by reference, and CDI (29 mg, 0.177 mmol) dissolved in dry CH₂Cl₂ (2 mL) under ice-water bath, DIEA (151 mg, 1.17 mmol) was added. The reaction mixture was stirred for 1 h at room temperature and added a solution of methyl 3-((5-chloro-2-methylphenyl)(piperidin-3-yl)amino)propylcarbamate (60 mg, 0.177 mmol) in dry CH₂Cl₂ (1 mL). The reaction mixture was stirred at room temperature overnight and then washed with water and brine, dried over Na₂SO₄, concentrated in vacuo to give the crude product, which was purified by preparative TLC to afford tert-butyl (S)-2-(3-((3-(methoxycarbonylamino)propyl)(5-chloro-2-methylphenyl)amino)piperidine-1-carboxamido)-3-cyclohexylpropyl(methyl)carbamate (80 mg, 82%).

### Step 2. methyl 3-((5-chloro-2-methylphenyl)((S)-1-((S)-1-cyclohexyl-3-(methylamino)propan-2-ylcarbamoyl)piperidin-3-yl)amino)propylcarbamate

tert-butyl (S)-2-(3-((3-(methoxycarbonylamino)propyl)(5-chloro-2-methylphenyl)amino)piperidine-1-carboxamido)-3-cyclohexylpropyl(methyl)carbamate (20 mg, 0.032 mmol) was dissolved in a solution of 20% (V/V) TFA/CH₂Cl₂ (2 mL). The reaction mixture was stirred at room temperature for 1 h, the solvent was removed under reduced pressure to give the crude product, which was purified by preparative HPLC to afford isomer 1 methyl 3-((5-chloro-2-methylphenyl)((S)-1-((S)-1-cyclohexyl-3-(methylamino)propan-2-ylcarbamoyl)piperidin-3-yl)amino)propylcarbamate (7 mg, 16%) and isomer 2 methyl 3-((5-chloro-2-methylphenyl)((R)-1-((S)-1-cyclohexyl-3-(methylamino)propan-2-ylcarbamoyl)piperidin-3-yl)amino)propylcarbamate (2.06 mg, 5%). Isomer 1: ¹H NMR (MeOD, 400 MHz) δ 0.7-1.05 (m, 3H), 1.1-1.32 (m, 5H), 1.35-1.52 (m, 4H), 1.52-1.82 (m, 7H), 1.95 (m, 1H), 2.28 (s, 3H), 2.68 (s, 3H), 2.7-2.94 (m, 4H), 3.0 (m, 1H), 3.05 (m, 2H), 3.15 (m, 2H), 3.6 (m, 3H), 3.92 (m, 1H), 4.06 (m, 2H), 7.0 (s, 1H), 7.2 (m, 2H). Isomer 2: ¹H NMR (MeOD, 400 MHz) δ 0.7-1.05 (m, 2H), 1.12-1.26 (m, 3H), 1.30 (m, 2H), 1.40 (m, 1H), 1.4-1.51 (m, 4H), 1.52-1.82 (m, 8H), 1.90-2.0 (m, 1H), 2.28 (s, 3H), 2.68 (s, 3H), 2.7-2.90 (m, 4H), 3.0-3.1 (m, 4H), 3.1-3.28 (m, 2H), 3.6 (m, 3H), 3.79 (m, 1H), 4.1 (m, 1H), 4.26 (m, 1H), 7.0 (s, 1H), 7.2 (m, 2H).

The following compounds were prepared following procedures analogous to those described above
1) methyl 3-((3-chlorophenyl)(1-((S)-1-cyclohexyl-3-(methylamino)propan-2-ylcarbamoyl)piperidin-3-yl)amino)propylcarbamate using methyl 3-((3-chlorophenyl)(piperidin-3-yl)amino)propylcarbamate in Step 1.
2) methyl 3-((3-chlorophenyl)((R)-1-((S)-1-(methylamino)-3-((R)-tetrahydro-2H-pyran-3-yl)propan-2-ylcarbamoyl)piperidin-3-yl)amino)propylcarbamate using methyl 3-((3-chlorophenyl)(piperidin-3-yl)amino)propylcarbamate and tert-butyl (S)-2-amino-3-((R)-tetrahydro-2H-pyran-3-yl)propyl(methyl)carbamate in Step 1.

### Example 2

### (S)-Methyl 3-((3-chlorophenyl)(3-(1-cyclohexyl-3-(methylamino)propan-2-ylcarbamoyl)phenyl)amino)propylcarbamate

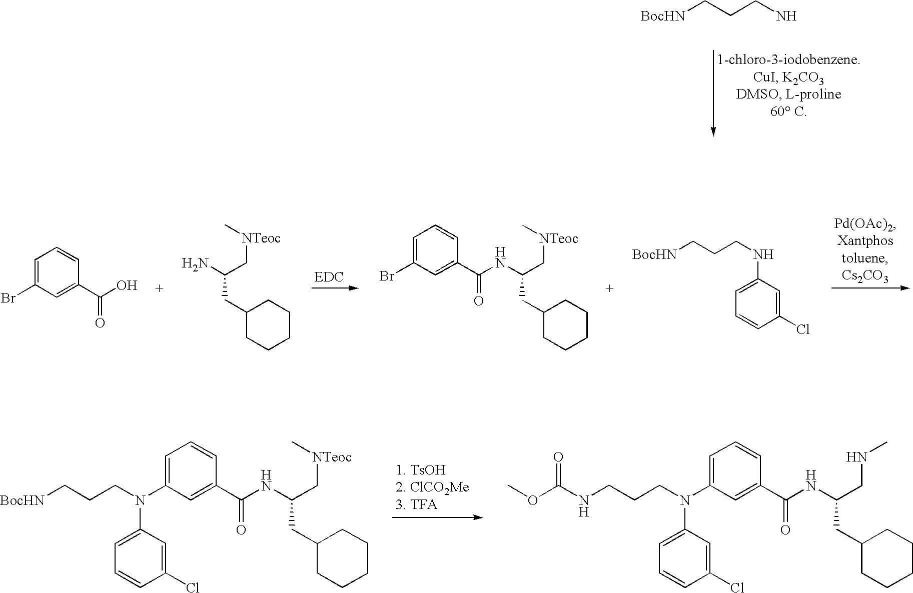

### Step 1. (S)-2-(trimethylsilyl)ethyl 2-(3-bromobenzamido)-3-cyclohexylpropyl(methyl)carbamate

A mixture of 3-bromobenzoic acid (629.0 mg, 3.13 mmol, 1.0 equiv), (S)-2-(trimethylsilyl)ethyl 2-amino-3-cyclohexylpropyl(methyl)carbamate (1.10 g, 3.49 mmol, 1.11 equiv), EDC (1.29 g, 2.15 equiv), and DIEA (4 mL) in CH₂Cl₂ (20 mL) was stirred at room temperature for 2 d. After evaporation of solvent, the residue was purified by chromatography on silica gel eluted with hexanes/ethyl acetate to afford (S)-2-(trimethylsilyl)ethyl 2-(3-bromobenzamido)-3-cyclohexylpropyl(methyl)carbamate. ESI MS: 497 (M+H⁺).

### Step 2. tert-butyl 3-(3-chlorophenylamino)propylcarbamate

A mixture of 1-chloro-3-iodobenzene (1.51 g, 6.32 mmol, 1.0 equiv), tert-butyl 3-aminopropylcarbamate (1.20 g, 6.89 mmol, 1.09 equiv), K₂CO₃ (2.16 g, 15.64 mmol, 2.47 equiv), CuI (0.14 g, 0.72 mmol, 0.11 equiv), and L-proline (0.30 g, 2.58 mmol, 0.41 equiv) in DMSO was heated to 60° C. for 24 h. The cooled mixture was partitioned between water and ethyl acetate. The organic layer was separated and the aqueous layer was extracted with ethyl acetate. The combined organic layers were washed with brine, dried over Na₂SO₄, and concentrated in vacuo. The residue was purified by chromatography on silica gel eluted with hexanes/ethyl acetate to give 724 mg (40%) of tert-butyl 3-(3-chlorophenylamino)propylcarbamate. ESI MS: 285 (M+H⁺).

### Step 3. (S)-2-(trimethylsilyl)ethyl 2-(3-((3-tert-butoxycarbonylaminopropyl)(3-chlorophenyl)amino)benzamido)-3-cyclohexylpropyl(methyl)carbamate

A 100 mL flask was charged with (S)-2-(trimethylsilyl)ethyl 2-(3-bromobenzamido)-3-cyclohexylpropyl(methyl)carbamate (85.3 mg, 0.17 mmol, 1.0 equiv), tert-butyl 3-(3-chlorophenylamino)propylcarbamate (65.9 mg, 0.23 mmol, 1.35 equiv), Pd(OAc)₂ (33 mg, 0.085 equiv), Xantphos (123 mg, 0.124 equiv), and toluene (2 mL). After the flask was degassed and refilled with nitrogen, the mixture was stirred at room temperature for 5 min and then Cs₂CO₃ (190 mg, 0.58 mmol, 3.4 equiv) was added. The reaction mixture was heated at 86° C. for 15 h and then cooled to room temperature, diluted with saturated brine, extracted with ethyl acetate (3×), and dried over Na₂SO₄. After the solvent was evaporated, the residue was purified by reversed-phase HPLC (Phenomenex® Luna 5μ C18(2) 100 A, 250×21.20 mm, 5 micron, 50%→90% CH₃CN/H₂O, 0.1% CF₃COOH over 15 min and then 90% CH₃CN/H₂O, 0.1% CF₃COOH over 3 min, flow rate 25 mL/min) to give 64.6 mg (54%) of (S)-2-(trimethylsilyl)ethyl 2-(3-((3-tert-butoxycarbonylaminopropyl)(3-chlorophenyl)amino)benzamido)-3-cyclohexylpropyl(methyl)carbamate. ESI MS: 701 (M+H⁺).

### Step 4. (S)-2-(trimethylsilyl)ethyl 2-(3-((3-aminopropyl)(3-chlorophenyl)amino)benzamido)-3-cyclohexylpropyl(methyl)carbamate

A mixture of (S)-2-(trimethylsilyl)ethyl 2-(3-((3-tert-butoxycarbonylaminopropyl)(3-chlorophenyl)amino)benzamido)-3-cyclohexylpropyl(methyl)carbamate (64.6 mg, 0.092 mmol, 1.0 equiv) and p-toluenesulfonic acid monohydrate (21.2 mg, 0.11 mmol, 1.2 equiv) in MeOH was heated with a 60° C. water bath. After the solvent was removed under reduced pressure, the crude product was used in the next step without further purification. ESI MS: 601 (M+H⁺).

### Step 5. (S)-2-(trimethylsilyl)ethyl 2-(3-((3-chlorophenyl)(3-methoxycarbonylaminopropyl)amino)benzamido)-3-cyclohexylpropyl(methyl)carbamate

A mixture of (S)-2-(trimethylsilyl)ethyl 2-(3-((3-aminopropyl)(3-chlorophenyl)amino)benzamido)-3-cyclohexylpropyl(methyl)carbamate, obtained as described above, 66.6 mg (5.9 equiv) of 4-dimethylaminopyridine, 2 mL of triethylamine, and 468.0 mg (54 equiv) of methyl chloroformate in CH₂Cl₂ (10 mL) was stirred at room temperature for 2 d. After the solvents were removed in vacuo, the residue was purified by reversed-phase HPLC (Phenomenex® Luna 5μ C18(2) 100 A, 250×21.20 mm, 5 micron, 50%—90% CH₃CN/H₂O, 0.1% CF₃COOH over min and then 90% CH₃CN/H₂O, 0.1% CF₃COOH over 3 min, flow rate 25 mL/min) to afford (S)-2-(trimethylsilyl)ethyl 2-(3-((3-chlorophenyl)(3-methoxycarbonylaminopropyl)amino)benzamido)-3-cyclohexylpropyl(methyl)carbamate. ESI MS: 659 (M+H⁺).

### Step 6. (S)-methyl 3-((3-chlorophenyl)(3-(1-cyclohexyl-3-(methylamino)propan-2-ylcarbamoyl)phenyl)amino)propylcarbamate

A mixture of (S)-2-(trimethylsilyl)ethyl 2-(3-((3-chlorophenyl)(3-methoxycarbonylaminopropyl)amino)benzamido)-3-cyclohexylpropyl(methyl)carbamate, obtained as described above, and trifluoroacetic acid (2 mL) in CH₂Cl₂ (5 mL) was stirred at room temperature for 1 h. After the solvents were removed in vacuo, the residue was purified by reversed-phase HPLC (Phenomenex® Luna 5 C18(2) 100 A, 250×21.20 mm, 5 micron, 10%→90% CH₃CN/H₂O, 0.1% CF₃COOH over 13 min, flow rate 25 mL/min) to give TFA salt of (S)-methyl 3-((3-chlorophenyl)(3-(1-cyclohexyl-3-(methylamino)propan-2-ylcarbamoyl)phenyl)amino)propylcarbamate. ESI MS: 515 (M+H⁺); ¹H NMR (400 MHz, CD₃OD) δ 7.52-7.48 (m, 2H), 7.40 (t, J=7.9 Hz, 1H), 7.23-7.14 (m, 2H), 6.85-6.81 (m, 3H), 4.46-4.43 (m), 3.76-3.72 (m), 3.56 (s, 3H), 3.14-3.10 (m), 3.02-2.96 (m), 2.66 (s, 3H), 1.81-0.82 (m).

The following are compounds of the invention. Compound names were generated with the assistance of ChemDraw®versions 8.0 and 9.0 (CambridgeSoft Corporation, 100 CambridgePark Drive, Cambridge, Mass. 02140 USA). When the stereochemistry at a chiral center is not defined in the compound name this indicates that the sample prepared contained a mixture of isomers at this center.

Table of Compounds

| | | Synthetic | | | | |
|---|---|---|---|---|---|---|
| | | Method | LC_MS | | | Selected |
| | | Example | Method 1 | Mass | 1H NMR | 1H NMR |
| Cpd. No. | Name | No. | t_{R} (min) | observed | Solvent | resonances |
| I-1a | methyl 3-((3- | 1 | 2.089 | 510 | MeOD | 0.80 (m, |
| | chlorophenyl)(1-((S)-1- | | | | | 2H), |
| | cyclohexyl-3- | | | | | 1.05-1.4 (m, 5H), |
| | (methylamino)propan-2- | | | | | 1.50 (m, |
| | ylcarbamoyl)piperidin-3- | | | | | 1H), |
| | yl)amino)propylcarbamate | | | | | 1.70 (m, 6H), |
| | | | | | | 1.80 (m, |
| | | | | | | 1H), |
| | | | | | | 2.70 (m, 3H), |
| | | | | | | 2.90 (s, 3H), |
| | | | | | | 3.10 (m, |
| | | | | | | 4H), 3.61 (s, |
| | | | | | | 3H), |
| | | | | | | 3.65 (m, 1H), |
| | | | | | | 3.75 (m, |
| | | | | | | 2H), |
| | | | | | | 3.9-4.05 (m, |
| | | | | | | 1H), |
| | | | | | | 4.25 (m, |
| | | | | | | 1H), |
| | | | | | | 7.3 (m, 1H), |
| | | | | | | 7.5 (m, 3H) |
| I-2a | methyl 3-((5-chloro-2- | 1 | 1.831 | 524 | MeOD | 1.30 (m, |
| | methylphenyl)((S)-1-((S)- | | | | | 2H), |
| | 1-cyclohexyl-3- | | | | | 1.44 (m, 2H), |
| | (methylamino)propan-2- | | | | | 1.98 (m, |
| | ylcarbamoyl)piperidin-3- | | | | | 1H), 2.71 (s, |
| | yl)amino)propylcarbamate | | | | | 3H), |
| | | | | | | 3.12 (m, 4H), |
| | | | | | | 3.45 (m, |
| | | | | | | 1H), 3.67 (s, |
| | | | | | | 3H), |
| | | | | | | 3.85 (m, 2H), |
| | | | | | | 4.05 (m, |
| | | | | | | 2H), |
| | | | | | | 6.66 (m, 1H), |
| | | | | | | 6.81 (m, |
| | | | | | | 2H), |
| | | | | | | 7.13 (m, 1H) |
| I-2b | methyl 3-((5-chloro-2- | 1 | 1.824 | 524 | MeoD | 1.29 (m, |
| | methylphenyl)((R)-1-((S)- | | | | | 2H), |
| | 1-cyclohexyl-3- | | | | | 1.43 (m, 2H), |
| | (methylamino)propan-2- | | | | | 1.97 (m, |
| | ylcarbamoyl)piperidin-3- | | | | | 1H), 2.70 (s, |
| | yl)amino)propylcarbamate | | | | | 3H), |
| | | | | | | 2.85 (m, 3H), |
| | | | | | | 3.14 (m, |
| | | | | | | 4H), |
| | | | | | | 3.41 (m, 1H), |
| | | | | | | 3.63 (m, |
| | | | | | | 3H), |
| | | | | | | 3.86 (m, 3H), |
| | | | | | | 4.12 (m, |
| | | | | | | 2H), |
| | | | | | | 6.65 (m, 1H), |
| | | | | | | 6.80 (m, |
| | | | | | | 2H), |
| | | | | | | 7.13 (m, 1H) |
| I-3a | methyl 3-((3- | 1 | | 536 | MeOD | 0.94 (m, |
| | chlorophenyl)((R)-1-((S)- | | | | | 2H), |
| | 1-(methylamino)-3-((R)- | | | | | 1.96 (m, 1H), |
| | tetrahydro-2H-pyran-3- | | | | | 2.27 (s, 3H), |
| | yl)propan-2- | | | | | 2.67 (s, 3H), |
| | ylcarbamoyl)piperidin-3- | | | | | 2.81 (m, |
| | yl)amino)propylcarbamate | | | | | 4H), |
| | | | | | | 3.04 (m, 4H), |
| | | | | | | 3.16 (m, |
| | | | | | | 2H), 3.60 (s, |
| | | | | | | 3H), |
| | | | | | | 3.78 (m, 1H), |
| | | | | | | 4.08 (m, |
| | | | | | | 1H), |
| | | | | | | 4.26 (m, 1H), |
| | | | | | | 7.00 (m, |
| | | | | | | 1H), |
| | | | | | | 7.19 (m, 2H) |
| I-4a | (S)-methyl 3-((3- | 2 | | 515, | CD₃OD | 7.52-7.48 (m, 2H), |
| | chlorophenyl)(3-(1- | | | 517 | | 7.40 (t, J = 7.9 Hz, |
| | cyclohexyl-3- | | | (M + 1) | | 1H), |
| | (methylamino)propan-2- | | | | | 7.23-7.14 (m, 2H), |
| | ylcarbamoyl)phenyl)amino)propylcarbamate | | | | | 6.85-6.81 (m, 3H), |
| | | | | | | 4.46-4.43 (m), |
| | | | | | | 3.76-3.72 (m), |
| | | | | | | 3.56 (s, 3H), |
| | | | | | | 3.14-3.10 (m), |
| | | | | | | 3.02-2.96 (m), |
| | | | | | | 2.66 (s, 3H), |
| | | | | | | 1.81-0.82 (m). |

### Example 3

### In Vitro Activity Studies

### Ic₅₀ for Renin

The compounds of the invention have enzyme-inhibiting properties. In particular, they inhibit the action of the natural enzyme renin. The latter passes from the kidneys into the blood where it effects the cleavage of angiotensinogen, releasing the decapeptide angiotensin I, which is then cleaved in the blood, lungs, the kidneys and other organsby angiotensin converting enzyme to form the octapeptide angiotensin II. The octapeptide increases blood pressure both directly by binding to its receptor, causing arterial vasoconstriction, and indirectly by liberating from the adrenal glands the sodium-ion-retaining hormone aldosterone, accompanied by an increase in extracellular fluid volume. That increase can be attributed to the action of angiotensin II. Inhibitors of the enzymatic activity of renin bring about a reduction in the formation of angiotensin I. As a result, a smaller amount of angiotensin II is produced. The reduced concentration of that active peptide hormone is the direct cause of the hypotensive effect of renin inhibitors.

The action of renin inhibitors in vitro is demonstrated experimentally by means of a test that measures the increase in fluorescence of an internally quenched peptide substrate. The sequence of this peptide corresponds to the sequence of human angiotensinogen. The following test protocol is used: All reactions are carried out in a flat bottom white opaque microtiter plate. A 4 μL aliquot of 400 μM renin substrate (DABCYL-γ-Abu-Ile-His-Pro-Phe-His-Leu-Val-Ile-His-Thr-EDANS) in 192 μL assay buffer (50 mM BES, 150 mM NaCl, 0.25 mg/mL bovine serum albumin, pH7.0) is added to 4 μL of test compound in DMSO at various concentrations ranging from 10 μM to 1 nM final concentrations. Next, 100 μL of trypsin-activated recombinant human renin (final enzyme concentration of 0.2-2 nM) in assay buffer is added, and the solution is mixed by pipetting. The increase in fluorescence at 495 nm (excitation at 340 nm) is measured for 60-360 min at rt using a Perkin-Elmer Fusion microplate reader. The slope of a linear portion of the plot of fluorescence increases as a function of time is then determined, and the rate is used for calculating percent inhibition in relation to uninhibited control. The percent inhibition values are plotted as a function of inhibitor concentration, and the IC₅₀ is determined from a fit of this data to a four parameter equation. The IC₅₀ is defined as the concentration of a particular inhibitor that reduces the formation of product by 50% relative to a control sample containing no inhibitor. (Wang G. T. et al. Anal. Biochem. 1993, 210, 351; Nakamura, N. et al. J. Biochem. (Tokyo) 1991, 109, 741; Murakami, K. et al. Anal Biochem. 1981, 110, 232).

### Example 4

### IC₅₀ VALUES OF THE DISCLOSED COMPOUNDS FOR RENIN

The IC₅₀ values of the disclosed compounds for renin were determined according to the protocols described in Example 3. In these in vitro systems, the compounds of the invention exhibit 50% inhibition at concentrations of from approximately 5000 nM to approximately 0.01 nM. Preferred compounds of the invention exhibit 50% inhibition at concentrations of from approximately 50 n M to approximately 0.01 nM. More preferred compounds of the invention exhibit 50% inhibition at concentrations of from approximately 5 nM to approximately 0.01 nM. Highly preferred compounds of the invention exhibit 50% inhibition at concentrations of from approximately 5 nM to approximately 0.01 nM and exhibit 50% inhibition at concentrations of from approximately 10 nM to approximately 0.01 nM in the in vitro assay in the presence of human plasma described below.

### Example 5

### In Vitro Activity of the Disclosed Compounds in Human Plasma

The action of renin inhibitors in vitro in human plasma can be demonstrated experimentally by the decrease in plasma renin activity (PRA) levels observed in the presence of the compounds. Incubations mixtures will contain in the final volume 250 μL 95.5 mM N,N-bis(2-hydroxyethyl)-2-aminoethanesulfonic acid, pH 7.0, 8 mM EDTA, 0.1 mM neomycin sulfate, 1 mg/ml sodium azide, 1 mM phenylmethanesulfonyl fluoride, 2% DMSO and 87.3% of pooled mixed-gender human plasma stabilized with EDTA. For plasma batches with low PRA (less than 1 ng/ml/hr)-2 μM of recombinant human renin will be added to achieve PRA of 3-4 ng/ml/hr. The cleavage of endogenous angiotensinogen in plasma will be carried out at 37° C. for 90 min and the product angiotensin I is measured by competitive radioimmunoassay using DiaSorin PRA kit. Uninhibited incubations containing 2% DMSO and fully inhibited controls with 2 μM of isovaleryl-Phe-Nle-Sta-Ala-Sta-OH will be used for deriving percent of inhibition for each concentration of inhibitors and fitting dose-response data into a four parametric model from which IC₅₀ values, defined as concentrations of inhibitors at which 50% inhibition occurs, will be determined.

### Example 6

### Efficacy of the Disclosed Inhibitors in a Transgenic Rat Model

The efficacy of the renin inhibitors may also be evaluated in vivo in double transgenic rats engineered to express human renin and human angiotensinogen (Bohlender J, Fukamizu A, Lippoldt A, Nomura T, Dietz R, Menard J, Murakami K, Luft F C, Ganten D. High human renin hypertension in transgenic rats. Hypertension 1997, 29, 428-434).

Experiments could be conducted in 5-10 week-old double transgenic rats (dTGRs). The model has been described in detail earlier. Briefly, the human renin construct that may be used to generate transgenic animals (hRen) is made up of the entire genomic human renin gene (10 exons and 9 introns), with 3.0 kB of the 5′-promoter region and 1.2 kB of 3′ additional sequences. A human angiotensinogen construct containing the entire human angiotensinogen gene (5 exons and 4 introns), with 1.3 kB of 5′-flanking and 2.4 kB of 3′-flanking sequences may be used to generate rats producing human angiotensinogen (hAogen). The hRen and hAogen rats may be rederived using embryo transfer from breeding pairs obtained under license from Ascencion Gmbh (Germany). The hAogen and hRen may then be crossed to produce the double transgenic dTGR) off-spring. The dTGr rats should be maintained on irradiated rodent chow (5VO2, Purina Mills Inc) and normal water. Radio telemetry transmitters (TA 11PAC40, Data Sciences International) may be surgically implanted at 5-6 weeks of age. The telemetry system can provide 24-h recordings of systolic, mean, diastolic arterial pressure (SAP, MAP, DAP, respectively) and heart rate (HR). Prior to dosing, baseline hemodynamic measures should be obtained for 24 hours. Rats may then be dosed orally with vehicle or drug and monitored up to 48 hours post-dose.

### Example 7

### In Vivo Activity

The cardiac and systemic hemodynamic efficacy of selective renin inhibitors can be evaluated in vivo in sodium-depleted, normotensive cynomolgus monkeys and in sodium-depleted, normotensive beagle dogs following a single oral and intravenous administration of the test compound. Arterial blood pressure is monitored by telemetry in freely moving, conscious animals.

Cynomolgus Monkey: Six male naïve cynomolgus monkeys weighing between 2.5 and 3.5 kg can be used in the studies. At least 4 weeks before the experiment, the monkeys are anesthetized with ketamine hydrochloride (15 mg/kg, i.m.) and xylazine hydrochloride (0.7 mg/kg, i.m.), and are implanted into the abdominal cavity with a transmitter (Model #TL11M2-D70-PCT, Data Sciences, St. Paul, Minn.). The pressure catheter is inserted into the lower abdominal aorta via the femoral artery. The bipotential leads are placed in Lead II configuration. The animals are housed under constant temperature (19-25° C.), humidity (>40%) and lighting conditions (12 h light and dark cycle), are fed once daily, and are allowed free access to water. The animals are sodium depleted by placing them on a low sodium diet (0.026%, Expanded Primate Diet 829552 MP-VENaCl (P), Special Diet Services, Ltd., UK) 7 days before the experiment and furosemide (3 mg/kg, intramuscularly i.m., Aventis Pharmaceuticals) is administered at −40 h and −16 h prior to administration of test compound.

For oral dosing, the renin inhibitors are formulated in 0.5% methylcellulose at dose levels of 10 and 30 mg/kg (5 mL/kg) by infant feeding tubes. For intravenous delivery, a silastic catheter is implanted into posterior vena cava via a femoral vein. The catheter is attached to the delivery pump via a tether system and a swivel joint. Test compound (dose levels of 0.1 to 10 mg/kg, formulated at 5% dextrose) is administered by continuous infusion (1.67 mL/kg/h) or by bolus injection (3.33 mL/kg in 2 min).

Arterial blood pressures (systolic, diastolic and mean) and body temperature are recorded continuously at 500 Hz and 50 Hz, respectively, using the Dataquest™ A.R.T. (Advanced Research Technology) software. Heart rate is derived from the phasic blood pressure tracing. During the recording period, the monkeys are kept in a separate room without human presence to avoid pressure changes secondary to stress. All data are expressed as mean±SEM. Effects of the renin inhibitors on blood pressure are assessed by ANOVA, taking into account the factors dose and time compared with the vehicle group.

Beagle Dogs: Non-naive Beagle dogs (2 per sex) weighing between 9 and 11 kg can be used in the studies. Each animal is implanted subcutaneously with a telemetry transmitter (Data Sciences) and the blood pressure catheter is inserted into the left femoral artery. The electrocardiogram leads are also tunneled subcutaneously to the appropriate anatomical regions. The animals are housed under constant temperature and lighting conditions, are fed once daily, and are allowed free access to water. A sodium depleted state is produced by placing them on a low-sodium diet (<4 meq/day, a combination of canned Prescription Diet canine h/d, from Hill's Pet Products and dry pellets from Bio-Serv Inc., Frenchtown, N.J.) beginning 10 days before the experiment, and furosemide (3 mg/kg i.m.; Aventis Pharmaceuticals) is administered at −40 and −16 h prior to administration of test compound.

A renin inhibitor is orally administered by orogastric gavage to all overnight fasted animals at a dose level of 30 mg/kg (4 mL/kg formulated in 0.5% methylcellulose). Food is given 4 h postdose. In some experiments, the renin inhibitor is administered by bolus i.v. at increasing dose levels of 1, 3 and 6 mg/kg (2, 6 and 20 mg/mL formulated in sterile saline). Cardiovascular parameters are collected continuously at least 80 min predose and 3 h postdose, followed by every min for 5 h and every 30 min for 16 h postdose. The Dataquest™ ART (version 2.2) software package from DSI (Data Sciences International) is used to collect telemetered cardiovascular data.

While this invention has been particularly shown and described with references to preferred embodiments thereof, it will be understood by those skilled in the art that various changes in form and details may be made therein without departing from the scope of the invention encompassed by the appended claims.

1. A compound represented by the following structural formula (I): 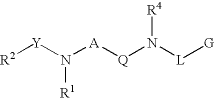
or an enantiomer, diastereomer or a pharmaceutically acceptable salt thereof, wherein:
R¹ is (C₃-C₇)cycloalkyl, phenyl, heteroaryl, or bicyclic heteroaryl each optionally substituted with 1 to 3 groups independently selected from:
fluorine, chlorine, bromine, cyano, nitro, hydroxyl, (C₁-C₆)alkyl, (C₃-C₆)cycloalkyl, (C₄-C₇)cycloalkylalkyl, (C₂-C₆)alkenyl, (C₅-C₇)cycloalkylalkenyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, halo(C₂-C₆)alkenyl, halo(C₃-C₆)alkynyl, halo(C₅-C₇)-cycloalkylalkynyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy and (C₁-C₆)alkanesulfonyl; and phenyl, heteroaryl, phenoxy, heteroaryloxy, phenylthio, heteroarylthio, benzyl, heteroarylnmethyl, benzyloxy and heteroarylmethoxy, each optionally substituted with 1 to 3 groups independently selected from: fluorine, chlorine, bromine, cyano, nitro, hydroxyl, (C₁-C₃)alkyl, halo(C₁-C₃)alkyl, (C₁-C₃)-alkoxy, and halo(C₁-C₃)alkoxy, and aminocarbonyl;
R² is (C₁-C₁₂)alkyl, (C₂-C₁₂)alkenyl, (C₂-C₁₂)alkynyl, (C₃-C₇)cycloalkyl, R^{2a}—O—, R^{2d}—O—, R^{2a}—S—, R^{2a}—O—Y₁—, R^{2a}—S—Y₁, R^{2a}—O—Y₁—O—, R^{2a}—O—Y₁—S—, R^{2a}—S—Y₁—O—, R^{2a}—S—Y₁—S—, R^{2a}—S—Y₁—O—Y₁—, NH₂—C(O)—NH—Y₁—, NH₂—C(O)—NH—Y—O—, NH₂—C(O)—NH—Y₁—S—, R^{2a}—C(O)—NH—Y₁—, R^{2a}—C(O)—NH—Y₁—O—, R^{2a}—C(O)—NH—Y₁—S—, NH₂—S(O)₂—NH—Y₁—, NH₂—S(O)₂—NH—Y₁—, NH₂—S(O)₂—N—Y₁—S—, R^{2a}—S(O)₂—NH—Y₁—, R^{2a}—S(O)₂—NH—Y₁—O—, R^{2a}—S(O)₂—NH—Y₁—S—, H—C(O)—NH—, H—C(O)—NH—Y₁—, H—C(O)—NH—Y₁—O—, H—C(O)—NH—Y₁—S—, R^{2a}—O—C(O)—NH—, R^{2a}—O—C(S)—NH—, R^{2a}—O—C(O)—NH—Y₁—, R^{2a}—O—C(O)—NH—Y₁—, R^{2a}—C(O)—NH—Y —S—, R^{2a}—NH—C(O)—NH—Y₁—, (R^{2a})₂—N—C(O)—NH—Y₁—, R^{2a}—NH—C(O)—NH—Y₁—O—, (R^{2a})₂—N—C(O)—NH—Y₁—O—, R^{2a}—NH—C(O)—NH—Y₁—S—, (R^{2a})₂—N—C(O)—NH—Y, —S—, NH₂—C(O)—, NH₂—C(S)—, NH₂—C(O)—Y₁—, NH₂—C(O)—Y₁—, NH₂—C(O)—Y₁—S—, R^{2a}—NH—C(O)—, R^{2a}—NH—C(S)—, R^{2a}—NH—C(O)—Y₁—, R^{2a}—NH—C(O)—Y—O—, R^{2a}—NH—C(O)—Y₁—S—, NH₂—C(O)—O—, NH₂—C(S)—O—, NH₂—C(O)—O—Y₁, NH₂—C(O)—O—Y₁—O—, NH₂—C(O)—O—Y₁—S—, R^{2a}—NH—C(O)—O—, R^{2a}—NH—C(S)—O—, R^{2a}—NH—C(O)—O—Y₁—, R^{2a}—NH₂—C(O)—O—Y₁—O—, R^{2a}—NH—C(O)—O—Y₁—S—, R^{2a}—NH—C(O)—NH—, R^{2a}—C(O)—NH—, NH₂—C(S)—S—, NH₂—C(O)—, R^{2a}—NH—C(S)—S—, R^{2a}—NH—C(O)—S—, R^{2a}—S—C(S)—NH—, R^{2a}—S—C(O)—NH—, R^{2a}—C(O)—, R^{2a}—C(S)—, NHC(═NR^{2e})(NH₂), NHC(═NR^{2e})(NHR^{2a}), 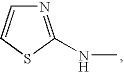
and
R^{2a} is straight or branched (C₁-C₁₂)alkyl, straight or branched (C₁-C₂)haloalkyl, (C₃-C₂)cycloalkyl, or straight or branched C₁-C₁₂ alkoxyalkyl, R^{2d}=(C₂-C₁₂)alkenyl, and R^{2e} is H, (C₁-C₆)alkyl, phenyl, heteroaryl, cyano, nitro, —S(O)R^{2a}—S(O)₂R^{2a}, —S(O)₂NHR^{2a}, —S(O)₂NR^{2a}R^{2a}, —C(O)R^{2a}, —C(S)R^{2a}, —C(O)OR^{2a}, —C(S)OR^{2a}, —C(O)(NHI-2), —C(O)(NHR^{2a}), Y₁ is a straight or branched C₁-C₁₂ alkylene optionally substituted with one or more halogens and optionally interrupted at an internal carbon atom with oxygen;
R² can be substituted by at least one of:
a) 1 to 6 halogen atoms; or
b) one substituent selected from the group consisting of cyano, hydroxyl, (C₁-C₃)alkoxy, (C₃-C₆)cycloalkyl, (C₃-C₆)cycloalkoxy, halo(C₁-C₃)alkoxy, halo(C₃-C₆)cycloalkyl and halo(C₃-C₆)cycloalkoxy; and
wherein the thio-moiety of said unsubstituted or substituted R^{2a}—S—, R^{2a}—S—Y₁—, R^{2a}—O—Y₁—S—, R^{2a}—S—Y₁—O—, R^{2a}—S—Y₁—S—, NH₂—C(O)—NH—Y₁—S—, R^{2a}—C(O)—NH—Y₁—S—, NH₂—S(O)₂—NH—Y₁—S—, R^{2a}—S(O)₂—NH—Y₁—S—, H—C(O)—NH—Y₁—S—, R^{2a}—O—C(O)—NH—Y₁—S—, R^{2a}—NH—C(O)—NH—Y₁—S—, (R^{2a})₂—N—C(O)—NH—Y —S—, NH₂—C(O)—Y —S—, —R^{2a}—NH—C(O)—Y —S—, NH₂—C(O)—O—Y₁—S—, R^{2a}—NH—C(O)—O—Y, —S—, NH₂—C(S)—S—, NH₂—C(O)—S—, R^{2a}—NH—C(S)—S—, R^{2a}—NH—C(O)—S—, R^{2a}—S—C(S)—NH—, R^{2a}—S—C(O)—NH—, and 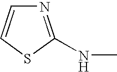
is optionally replaced by —S(O)— or —S(O)₂—; and
wherein the carbonyl moiety of said unsubstituted or substituted NH₂—C(O)—NH—Y₁, NH₂—C(O)—NH—Y₁—O—NH₂—C(O)—NH—Y₁—S—, R^{2a}—C(O)—NH—Y₁—, R^{2a}—C(O)—NH—Y₁—O—, R^{2a}—C(O)—NH—Y₁—S—, H—C(O)—NH—, H—C(O)—NH—Y₁—, H—C(O)—NH—Y₁—O—, H—C(O)—NH—Y₁—S—, R^{2a}—O—C(O)—NH—, R^{2a}—O—C(O)—NH—Y₁—, R^{2a}—O—C(O)—NH—Y—O—, R^{2a}—O—C(O)—NH—Y₁—S—, R^{2a}—NH—C(NH—H(O)—N—Y₁—, (R^{2a})₂—N—C(O)—NH—Y₁—, R^{2a}—NH—C(O)—NH—Y₁—O—, (R^{2a})₂—N—C(O)—NH—Y₁—O—, R^{2a}—NH—C(O)—NH—Y₁—S—, (R^{2a})₂—N—C(O)—NH—Y₁—S—, NH₂—C(O)—, NH₂—C(O)—Y₁—, NH₂—C(O)—Y₁—O—, NH₂—C(O)—Y₁—S—, R^{2a}—NH—C(O)—, R^{2a}—NH—C(O)—Y₁—, R^{2a}—NH—C(O)—Y, —O—, R^{2a}—NH—C(O)—Y₁—S—, NH₂—C(O)—O—, NH₂—C(O)—O—Y₁—, NH₂—C(O)—O—Y₁—O—, NH₂—C(O)—O—Y₁—S—, R^{2a}—NH—C(O)—O—, R^{2a}—NH—C(O)—O—Y₁—, R^{2a}—NH—C(O)—O—Y₁—O—, R^{2a}—NH—C(O)—O—Y₁—S—, R^{2a}—NH—C(O)—NH—, R^{2a}—C(O)—NH—, NH₂—C(O)—S—, R^{2a}—NH—C(O)—S—, R^{2a}—S—C(O)—NH—, and R^{2a}—C(O)—
is optionally replaced by a thiocarbonyl moiety;
provided that when R₂ is attached to 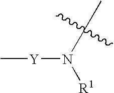
through a heteroatom, Y can not be a covalent bond;
A is a saturated or unsaturated 4-, 5-, 6-, or 7-membered ring which is optionally bridged by (CH₂)ₚ via bonds to two members of said ring, wherein said ring is composed of carbon atoms and 0-2 hetero atoms selected from the group consisting of 0, 1, or 2 nitrogen atoms, 0 or 1 oxygen atoms, and 0 or 1 sulfur atoms, said ring being optionally and independently substituted with zero to four halogen atoms, (C₁-C₆)alkyl groups, halo(C₁-C₆)alkyl groups or oxo groups such that when there is substitution with one oxo group on a carbon atom it forms a carbonyl group, and when there is substitution of one or two oxo groups on sulfur it forms sulfoxide or sulfone groups, respectively;
p is 1 to 3;
Y is a covalent bond or C₁-C₁₀ alkylene, C₁-C₁₀ alkenylene or C₁-C₁₀ alkynylene, each optionally substituted at one or more substitutable carbon atoms with halogen, cyano, hydroxyl, (C₁-C₃)alkyl, (C₁-C₃)alkoxy or halo(C₁-C₃)alkoxy,
Q is Q1, Q2, Q3, Q4, Q5, or Q6: 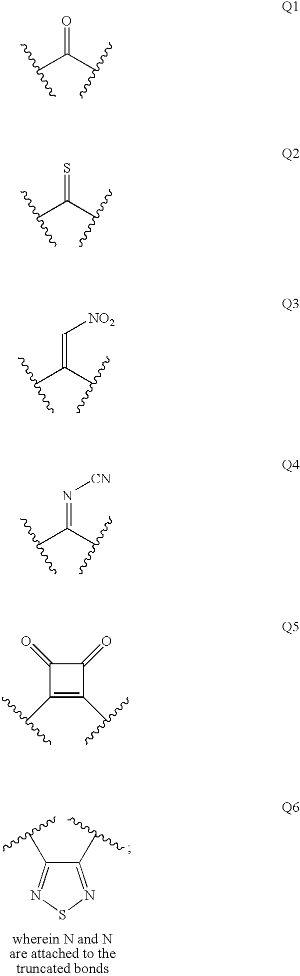
R⁴ is H, (C₁-C₆)alkyl, halo(C₁-C₆)alkyl; (C₁-C₃)alkoxy(C₁-C₃)alkyl, or cyano(C₁-C₆)alkyl;
G is OH, OR^{e}, NH₂, NHR^{e}, NR^{e}R^{f}, C(═NH)NH₂, C(═NH)NHR^{e}, NHC(═NH)NH₂, or NHC(═NH)NHR^{e};
L is 1) a linear (C₂-C₄)alkyl chain when G is OH, OR^{e}, NH₂, NHR^{e}, NR^{e}R^{f}, NHC(═NH)NH₂, or NHC(═NH)NHR^{e}, or 2) a linear (C₁-C₃)alkyl chain when G is C(═NH)NH₂ or C(═NH)NHR^{e};
L is optionally substituted by 1-4 groups independently selected from R⁵, R^{5a}, R⁶, and R^{6a}; one or more of the carbon atoms of L may be part of a 3-, 4-, 5-, 6-, or 7-membered saturated ring composed of carbon atoms, and 0-2 hetero atoms selected from 0 or 1 nitrogen atoms, 0 or 1 oxygen atoms, and 0 or 1 sulfur atoms; said saturated ring being optionally substituted with up to four groups selected from halogen, (C₁-C₆)alkyl, halo(C₁-C₆)alkyl, (C₃-C₆)cycloalkyl, halo(C₃-C₆)cycloalkyl, (C₄-C₇)cycloalkylalkyl, halo(C₄-C₇)cycloalkylalkyl, and oxo, such that when there is substitution with one oxo group on a carbon atom it forms a carbonyl group and when there is substitution of one or two oxo groups on sulfur it forms sulfoxide or sulfone groups, respectively;
R⁵, R^{5a}, R⁶, and R^{6a} is each independently selected from 1) H, (C₁-C₁₂)alkyl, halo(C₁-C₁₂)alkyl, hydroxy(C₁-C₁₂)alkyl, (C₃-C₁₀)cycloalkyl, (C₃-C₁₀)cycloalkyl, (C₃-C₁₀)cycloalkylalkyl, halo(C₃-C₁₀)cycloalkylalkyl, hydroxy(C₃-C₁₀)cycloalkylalkyl, (C₁-C₂)alkyl(C₃-C₁₀)cycloalkylalkyl, halo(C₁-C₂)alkyl(C₃-C₁₀)cycloalkylalkyl, di(C₁-C₂)alkyl(C₃-C₁₀)cycloalkylalkyl, hydroxy(C₁-C₂)alkyl(C₃-C₁₀)cycloalkylalkyl, hydroxy di(C₁-C₂)alkyl(C₃-C₁)cycloalkylalkyl, (C₂-C₁₂)alkenyl, (C₅-C₈)cycloalkyl(C₁-C₃)alkenyl, (C₂-C₁₂)alkynyl, (C₃-C₈)cycloalkyl(C₁-C₃)alkynyl, (C₄-C₁₂)bicycloalkyl(C₁-C₃)alkyl, (C₈-C₁₄)tricycloalkyl(C₁-C₃)alkyl, (C₁-C₆)alkoxy(C₁-C₆)alkyl, halo(C₁-C₆)alkoxy(C₁-C₆)alkyl, (C₃-C₈)cycloalkoxy(C₁-C₃)alkyl, (C₁-C₆)alkylthio(C₁-C₆)alkyl, halo(C₁-C₆)alkylthio(C₁-C₆)alkyl, (C₃-C₈)cycloalkylthio(C₁-C₃)alkyl, saturated heterocyclyl, and saturated heterocyclyl(C₁-C₃)alkyl wherein (a) hydrogen atoms in these groups are optionally substituted by 1 to 6 groups independently selected from halogen, cyano, nitro, hydroxy, (C₁-C₆)alkyl, (C₁-C₆)alkoxy, (C₃-C₆)cycloalkyl, (C₃-C₇)cycloalkylalkyl, halo(C₃-C₇)cycloalkylalkyl, (C₂-C₆)alkenyl, halo(C₂-C₆)alkenyl, (C₃-C₇)cycloalkylalkenyl, (C₂-C₆)alkynyl, halo(C₂-C₆)alkynyl, (C₃-C₇)cycloalkylalkoxy, halo(C₃-C₇)cycloalkylalkoxy, (C₃-C₇)cycloalkoxy, halo(C₁-C₆)alkyl, (C₃-C₇)cycloalkylalkynyl, halo(C₃-C₇)cycloalkylalkynyl, halo(C₁-C₆)alkoxy, halo(C₃-C₇)cycloalkyl, halo(C₃-C₇)cycloalkoxy, (C₁-C₆)alkylsulfonyl, aminocarbonyl and wherein (b) divalent sulfur atoms are optionally oxidized to sulfoxide or sulfone;
or 2) phenyl, naphthyl, heteroaryl, phenyl(C₁-C₃)alkyl, phenoxymethyl, naphthyl(C₁-C₃)alkyl, and heteroaryl(C₁-C₃)alkyl, each optionally substituted with 1 to 3 groups independently selected from: halogen, cyano, nitro, amino, hydroxy, carboxy, (C₁-C₆)alkyl, (C₃-C₈)cycloalkyl, (C₄-C₇)cycloalkylalkyl, (C₂-C₆)alkenyl, halo(C₂-C₆)alkenyl, (C₃-C₆)cycloalkylalkenyl, (C₂-C₆)alkynyl, halo(C₂-C₆)alkynyl, (C₃-C₆)cycloalkyl-(C₂-C₄)alkynyl, halo(C₃-C₇)cycloalkylalkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₈)cycloalkylhio, (C₄-C₇)cycloalkylalkylthio, halo(C₁-C₆)alkylthio, halo(C₃-C₆)cycloalkylhio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₈)alkanesulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)-cycloalkylalkanesulfonyl, (C₁-C₆)alkylamino, di(C₁-C₆)alkylamino, (C₁-C₆)-alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)-cycloalkylalkoxy(C₁-C₆)alkyl, (C₁-C₈)alkylthio(C₁-C₆)alkyl, (C₃-C₈)cycloalkylhio(C₁-C₆)alkyl, (C₄-C₈)cycloalkylalkylthio(C₁-C₆)alkyl, halo(C₁-C₈)alkylthio(C₁-C₆)alkyl, halo(C₃-C₈)cycloalkylhio(C₁-C₆)alkyl, halo(C₄-C₈)-cycloalkylalkylthio(C₁-C₈)alkyl, (C₁-C₈)alkanesulfinyl(C₁-C₆)alkyl, (C₃-C₈)-cycloalkanesulfinyl(C₁-C₆)alkyl, (C₄-C₈)cycloalkyl-alkanesulfinyl(C₁-C₆)alkyl, halo(C₁-C₈)alkanesulfinyl(C₁-C₆)alkyl, halo(C₃-C₈)cycloalkanesulfinyl(C₁-C₆)alkyl, halo(C₄-C₈)cycloalkylalkanesulfiny(C₁-C₆)alkyl, (C₁-C₈)alkane-sulfonyl(C₁-C₆)alkyl, (C₃-C₈)cycloalkanesulfonyl(C₁-C₆)alkyl, (C₄-C₈)cycloalkylalkanesulfonyl(C₁-C₆)alkyl, halo(C₁-C₈)alkanesulfonyl(C₁-C₆)alkyl, halo(C₃-C₈)cycloalkanesulfonyl(C₁-C₆)alkyl, halo(C₄-C₈)cycloalkylalkane-sulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₈)alkyl, aminocarbonyl(C₁-C₆)alkyl, (C₁-C₈)alkylamino-carbonyl(C₁-C₆)alkyl, di(C₁-C₈)alkylaminocarbonyl(C₁-C₆)alkyl and (C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylamino-carboxy(C₁-C₆)alkyl and di(C₁-C₈)alkylaminocarboxy(C₁-C₆)alkyl, phenyl, naphthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, naphthyl(C₁-C₃)alkyl, heteroaryl(C₁-C₃)alkyl, and bicyclic heteroaryl(C₁-C₃)alkyl, wherein the aromatic and heteroaromatic groups are optionally substituted with 1 to 3 groups independently selected from fluorine, chlorine, cyano, (C₁-C₃)alkyl, halo(C₁-C₃)alkyl, (C₁-C₃)alkoxy, halo(C₁-C₃)-alkoxy, (C₁-C₃)alkanesulfonyl, and (C₁-C₃)alkoxycarbonyl;
R^{e} is a) (C₁-C₁₂)alkyl, (C₄-C₁₂)cycloalkylalkyl, halo(C₁-C₁₂)alkyl, halo(C₄-C₁₂)cycloalkylalkyl, (C₂-C₁₂)alkenyl, (C₈-C₁₂)cycloalkylalkenyl, halo(C₂-C₁₂)alkenyl, halo(C₅-C₂)cycloalkylalkenyl, (C₂-C₂)alkynyl, (C₅-C₁₂)cycloalkylalkynyl, halo(C₂-C₁₂)alkynyl, halo(C₈-C₁₂)cycloalkylalkynyl, (C₁-C₆)alkoxy(C₁-C₆)alkyl, halo(C₁-C₆)alkoxy(C₁-C₆)alkyl, (C₁-C₆)alkylthio(C₁-C₆)alkyl, halo(C₁-C₆)alkylthio(C₁-C₆)alkyl, (C₁-C₆)alkanesulfinyl(C₁-C₆)alkyl, halo(C₁-C₆)alkane-sulfinyl(C₁-C₆)alkyl, (C₁-C₆)alkanesulfonyl(C₁-C₆)alkyl, halo(C₁-C₆)alkanesulfonyl(C₁-C₆)alkyl, aminocarbonyl(C₁-C₆)alkyl, (C₁-C₆)alkylaminocarbonyl(C₁-C₆)alkyl, di(C₁-C₆)alkylamino-carbonyl(C₁-C₆)alkyl, cyano(C₁-C₆)alkyl, carboxy(C₁-C₆)alkyl, (C₁-C₆)alkoxycarbonyl(C₁-C₆)alkyl, saturated heterocyclyl, or saturated heterocyclyl(C₁-C₆)alkyl or b) phenyl, naphthyl, heteroaryl, phenyl(C₁-C₃)alkyl, naphthyl(C₁-C₃)alkyl, or heteroaryl(C₁-C₃)alkyl, each of a) and b) is optionally substituted by 1 to 3 groups independently selected from:
1) fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)cycloalkyl-(C₂-C₄)alkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₈)alkylthio, (C₃-C₆)cycloalkylhio, (C₄-C₇)cycloalkylalkylthio, halo(C₁-C₆)alkylthio, halo(C₃-C₆)cycloalkylhio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃-C₆)cycloalkane-sulfinyl, halo(C₄-C₇)cycloalkylalkanesulfinyl, (C₁-C₈)alkanesulfonyl, (C₃-C₆)cycloalkanesulfonyl, (C₄-C₇)cycloalkylalkanesulfonyl, halo(C₃-C₈)alkanesulfonyl, halo(C₃-C₈)cycloalkanesulfonyl, halo(C₄-C₇)-cycloalkylalkanesulfonyl, (C₁-C₆)alkylamino, di(C₁-C₆)alkylamino, (C₁-C₆)alkoxy-(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl and di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)-cycloalkylalkoxy(C₁-C₆)alkyl, (C₁-C₈)alkylthio(C₁-C₆)alkyl, (C₃-C₈)cycloalkylhio(C₁-C₆)alkyl, (C₄-C₈)cycloalkylalkylthio(C₁-C₆)alkyl, halo(C₁-C₈)alkylthio(C₁-C₆)alkyl, halo(C₃-C₈)cycloalkylhio(C₁-C₆)alkyl, halo(C₄-C₈)-cycloalkylalkylthio(C₁-C₆)alkyl, (C₁-C₈)alkanesulfinyl(C₁-C₆)alkyl, (C₃-C₈)-cycloalkanesulfinyl(C₁-C₆)alkyl, (C₄-C₈)cycloalkyl-alkanesulfinyl(C₁-C₆)alkyl, halo(C₁-C₈)alkanesulfinyl(C₁-C₆)alkyl, halo(C₃-C₈)cycloalkanesulfinyl(C₁-C₆)alkyl, halo(C₄-C₈)cycloalkylalkanesulfinyl(C₁-C₆)alkyl, (C₁-C₈)alkane-sulfonyl(C₁-C₆)alkyl, (C₃-C₈)cycloalkanesulfonyl(C₁-C₆)alkyl, (C₄-C₈)cycloalkylalkanesulfonyl(C₁-C₆)alkyl, halo(C₁-C₈)alkanesulfonyl(C₁-C₆)alkyl, halo(C₃-C₈)cycioalkanesulfonyl(C₁-C₆)alkyl, halo(C₄-C₈)cycloalkylalkane-sulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylamino-carbonyl(C₁-C₆)alkyl, di(C₁-C₈)alkylaminocarbonyl(C₁-C₆)alkyl(C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylamino-carboxy(C₁-C₆)alkyl and di(C₁-C₈)alkylaminocarboxy(C₁-C₆)alkyl; or 2) phenyl, naphthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, naphthyl(C₁-C₃)alkyl, heteroaryl(C₁-C₃)alkyl, and bicyclic heteroaryl(C₁-C₃)alkyl, each optionally substituted with 1 to 3 groups independently selected from fluorine, chlorine, cyano, (C₁-C₃)alkyl, halo(C₁-C₃)alkyl, (C₁-C₃)alkoxy, halo(C₁-C₃)alkoxy, (C₁-C₃)alkanesulfonyl, and (C₁-C₃)-alkoxycarbonyl; or
b) R^{e} is a saturated divalent radical composed of carbon atoms, and 0, 1 or 2 hetero atoms selected from 0 or 1 nitrogen atoms, 0 or 1 oxygen atoms, and 0 or 1 sulfur atoms that is attached to any core carbon atom on L to form a saturated 3-, 4-, 5-, 6-, or 7-membered L-G ring; said L-G ring being optionally substituted with 1 to 4 groups selected from halogen, fluorine, (C₁-C₈)alkyl, halo(C₁-C₈)alkyl, (C₃-C₈)cycloalkyl, halo(C₃-C₈)cycloalkyl, hydroxy(C₃-C₈)cycloalkyl, (C₃-C₉)cycloalkyl(C₁-C₃)alkyl, halo(C₃-C₈)cycloalkyl(C₁-C₃)alkyl, hydroxy(C₃-C₈)cycloalkyl(C₁-C₃)alkyl, (C₁-C₈)alkoxy, halo(C₁-C₈)alkoxy, (C₃-C₈)cycloalkoxy, halo(C₃-C₈)cycloalkoxy, hydroxy(C₃-C₈)cycloalkoxy, (C₁-C₈)alkoxy(C₁-C₃)alkyl, halo(C₁-C₈)alkoxy(C₁-C₃)alkyl, (C₃-C₈)cycloalkoxy(C₁-C₃)alkyl, halo(C₃-C₈)cycloalkoxy(C₁-C₃)alkyl, hydroxy(C₃-C₈)cycloalkoxy(C₁-C₃)alkyl, (C₃-C₈)cycloalkyl(C₁-C₃)alkoxy(C₁-C₃)alkyl, halo(C₃-C₈)cycloalkyl(C₁-C₃)alkoxy(C₁-C₃)alkyl, hydroxy(C₃-C₈)cycloalkyl(C₁-C₃)alkoxy(C₁-C₃)alkyl, (C₁-C₈)alkylthio, halo(C₁-C₈)alkylthio, (C₃-C₈)cycloalkylthio, halo(C₃-C₈)cycloalkylthio, hydroxy(C₃-C₈)cycloalkylthio, (C₃-C₈)cycloalkyl(C₁-C₃)alkylthio, halo(C₃-C₈)cycloalkyl(C₁-C₃)alkylthio, hydroxy(C₃-C₈)cycloalkyl(C₁-C₃)alkylthio, (C₁-C₈)alkylthio(C₁-C₃)alkyl, halo(C₁-C₈)alkylthio(C₁-C₃)alkyl, (C₃-C₈)cycloalkylthio(C₁-C₃)alkyl, halo(C₃-C₈)cycloalkylthio(C₁-C₃)alkyl, hydroxy(C₃-C₈)cycloalkylthio(C₁-C₃)alkyl, (C₃-C₈)cycloalkyl(C₁-C₃)alkylthio(C₁-C₃)alkyl, halo(C₃-C₈)cycloalkyl(C₁-C₃)alkylthio(C₁-C₃)alkyl, hydroxy(C₃-C₈)cycloalkyl(C₁-C₃)alkylthio(C₁-C₃)alkyl, heterocyclyl, and oxo;
R^{f} is (C₁-C₆)alkyl or halo(C₁-C₆)alkyl.

2. The compound of claim 1, wherein the compound is represented by the following structural formula: 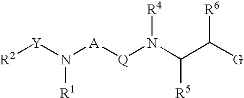
or an enantiomer, diastereomer or a pharmaceutically acceptable salt thereof.

3. The compound of claim 2, wherein:
G is OH, NH₂ or NHR; and
R^{e} is a) (C₁-C₆)alkyl, halo(C₁-C₆)alkyl, (C₄-C₁₀)cycloalkylalkyl, (C₁-C₅)alkoxy(C₁-C₅)alkyl, or aminocarbonyl(C₁-C₆)alkyl or b) phenyl(C₁-C₂)alkyl optionally substituted with 1 to 3 groups independently selected from: fluorine, chlorine, cyano, (C₁-C₃)alkyl, halo(C₁-C₃)alkyl, (C₁-C₃)alkoxy, and halo(C₁-C₃)alkoxy; or c) R⁵ and R^{e} together are —CH₂—, —(CH₂)₂—, —(CH₂)₃—, or —(CH₂)₄—, optionally substituted with 1 or 2 groups independently selected from fluorine, (C₁-C₈)alkyl, halo(C₁-C₈)alkyl, (C₃-C₆)cycloalkyl, halo(C₃-C₆)cycloalkyl, hydroxy(C₃-C₆)cycloalkyl, (C₃-C₆)cycloalkyl(C₁-C₂)alkyl, halo(C₃-C₆)cycloalkyl(C₁-C₂)alkyl, hydroxylated (C₃-C₆)cycloalkyl(C₁-C₂)alkyl, (C₁-C₈)alkoxy, halo(C₁-C₈)alkoxy, (C₃-C₆)cycloalkoxy, halo(C₃-C₆)cycloalkoxy, and heterocyclyl;
or a pharmaceutically acceptable salt thereof.

4. The compound of claim 3, wherein the compound is represented by the following structural formula: 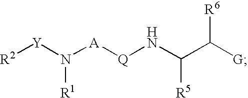
or an enantiomer, diastereomer or a pharmaceutically acceptable salt thereof.

5. The compound of claim 4, wherein the compound is represented by the following structural formula: 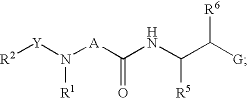
or an enantiomer, diastereomer or a pharmaceutically acceptable salt thereof.

6. The compound of claim 5, wherein the compound is represented by the following structural formula: 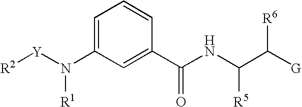
or an enantiomer, diastereomer or a pharmaceutically acceptable salt thereof.

7. The compound of claim 6, wherein, one of R⁵ and R⁶ is —H or methyl and the other is H, (C₁-C₁₀)alkyl, (C₄-C₁₀)cycloalkylalkyl, halo(C₁-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁₀)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₃)alkylthio(C₁-C₅)alkyl, halo(C₁-C₅)alkylthio(C₁-C₅)alkyl, or saturated heterocyclyl(C₁-C₃)alkyl, phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy.

8. The compound of claim 7, wherein R⁶ is —H or methyl.

9. The compound of claim 7, wherein R⁵ is —H or methyl.

10. The compound of claim 6, wherein the compound is represented by the following structural formula: 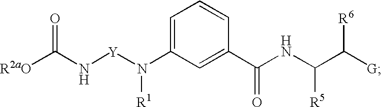
or an enantiomer, diastereomer or a pharmaceutically acceptable salt thereof.

11. The compound of claim 10, wherein the compound is represented by the following structural formula: 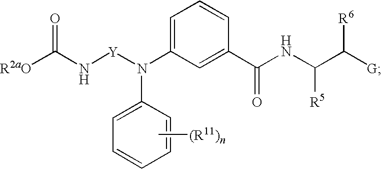
or an enantiomer, diastereomer or a pharmaceutically acceptable salt thereof, wherein:
R¹¹ is fluorine, chlorine, bromine, cyano, nitro, (C₁-C₆)alkyl, (C₃-C₆)cycloalkyl, (C₄-C₇)cycloalkylalkyl, (C₂-C₆)alkenyl, (C₅-C₇)cycloalkylalkenyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, halo(C₂-C₆)alkenyl, halo(C₃-C₆)alkynyl, halo(C₅-C₇)-cycloalkylalkynyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy and (C₁-C₆)alkanesulfonyl; or 2) phenyl, heteroaryl, phenoxy, heteroaryloxy, phenylthio, heteroarylthio, benzyl, heteroarylmethyl, benzyloxy and heteroarylmethoxy, each optionally substituted with 1 to 3 groups independently selected from: fluorine, chlorine, cyano, (C₁-C₃)alkyl, halo(C₁-C₃)alkyl, (C₁-C₃)-alkoxy, and halo(C₁-C₃)alkoxy, and aminocarbonyl;
n is 0, 1, 2 or 3.

12. The compound of claim 11, wherein:
R⁵ is (C₁-C₇)alkyl, halo(C₁-C₇)alkyl, hydroxy(C₁-C₇)alkyl, cyclohexylmethyl, halocyclohexylmethyl, hydroxylated cyclohexylmethyl, 2-(cyclohexyl)ethyl, (C₁-C₂)alkyl cyclohexylmethyl, di(C₁-C₂)alkyl cyclohexylmethyl, hydroxylated (C₁-C₂)alkyl cyclohexylmethyl, hydroxylated di(C₁-C₂)alkylcyclohexylmethyl, (3-noradamantyl)methyl, (tetrahydropyranyl)methyl, or oxepanyl methyl;
R⁶ is —H or methyl;
G is NH₂ or NHR^{e};
R^{e} is methyl or R⁵ and R^{e} together are —(CH₂)₃— optionally substituted with C₁-C₄ alkyl or cyclohexyl.

13. The compound of claim 11, wherein:
R⁶ is (C₁-C₇)alkyl, halo(C₁-C₇)alkyl, hydroxy(C₁-C₇)alkyl, cyclohexylmethyl, halocyclohexylmethyl, hydroxylated cyclohexylmethyl, 2-(cyclohexyl)ethyl, (C₁-C₂)alkyl cyclohexylmethyl, di(C₁-C₂)alkyl cyclohexylmethyl, hydroxylated (C₁-C₂)alkyl cyclohexylmethyl, hydroxylated di(C₁-C₂)alkylcyclohexylmethyl, (3-noradamantyl)methyl, (tetrahydropyranyl)methyl, or oxepanyl methyl;
R⁵ is —H or methyl;
G is NH₂ or NHR^{e};
R^{e} is methyl or R⁶ and R^{e} together are —(CH₂)₃— optionally substituted with C₁-C₄ alkyl or cyclohexyl.

14. The compound of claim 12, wherein:
R^{2a} is methyl or ethyl; and
R¹¹ is chloro, fluoro or methyl.

15. The compound of claim 13, wherein:
R^{2a} is methyl or ethyl; and
R¹¹ is chloro, fluoro or methyl.

16. The compound of claim 11, wherein the compound is represented by the following structural formula: 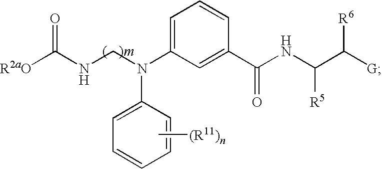
or an enantiomer, diastereorner or a pharmaceutically acceptable salt thereof, wherein m is 2 or 3.

17. The compound of claim 11, wherein the compound is represented by one of the following structural formula: 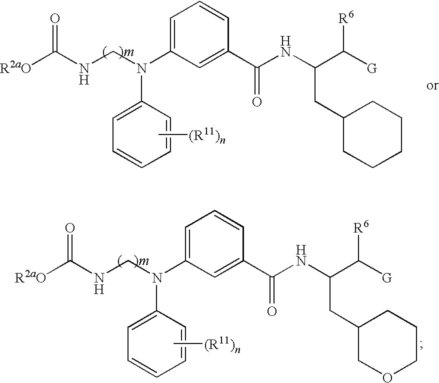
or an enantiomer, diastereomer or a pharmaceutically acceptable salt thereof.

18. The compound of claim 5, wherein the compound is represented by the following structural formula: 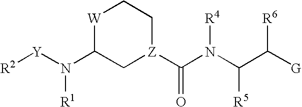
wherein: W═CH₂ or O and Z═CH or N or a pharmaceutically acceptable salt thereof.

19. The compound of claim 18, wherein the compound is represented by the following structural formula: 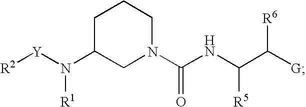
or an enantiomer, diastereomer or a pharmaceutically acceptable salt thereof.

20. The compound of claim 19, wherein:
one of R⁵ and R⁶ is —H1- or methyl and the other is H, (C₁-C₁₀)alkyl, (C₄-C₁₀)cycloalkylalkyl, halo(C₁-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁₀)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₅)alkyl, or saturated heterocyclyl(C₁-C₃)alkyl, phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy.

21. The compound of claim 20, wherein R⁶ is —H or methyl.

22. The compound of claim 20, wherein R⁵ is —H or methyl.

23. The compound of claim 19, wherein the compound is represented by the following structural formula: 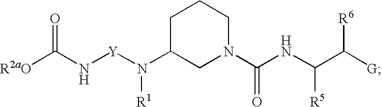
or an enantiomer, diastereomer or a pharmaceutically acceptable salt thereof.

24. The compound of claim 23, wherein the compound is represented by the following structural formula: 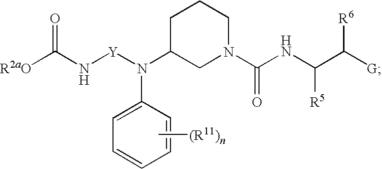
or an enantiomer, diastereomer or a pharmaceutically acceptable salt thereof, wherein:
R¹¹ is fluorine, chlorine, bromine, cyano, nitro, (C₁-C₆)alkyl, (C₃-C₆)cycloalkyl, (C₄-C₇)cycloalkylalkyl, (C₂-C₆)alkenyl, (C₅-C₇)cycloalkylalkenyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, halo(C₂-C₆)alkenyl, halo(C₃-C₆)alkynyl, halo(C₅-C₇)-cycloalkylalkynyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy and (C₁-C₆)alkanesulfonyl; or 2) phenyl, heteroaryl, phenoxy, heteroaryloxy, phenylthio, heteroarylthio, benzyl, heteroarylmethyl, benzyloxy and heteroarylmethoxy, each optionally substituted with 1 to 3 groups independently selected from: fluorine, chlorine, cyano, (C₁-C₃)alkyl, halo(C₁-C₃)alkyl, (C₁-C₃)-alkoxy, and halo(C₁-C₃)alkoxy, and aminocarbonyl;
n is 0, 1, 2 or 3.

25. The compound of claim 24, wherein:
R⁵ is (C1-C7)alkyl, halo(C1-C7)alkyl, hydroxy(C1-C7)alkyl, cyclohexylmethyl, halocyclohexylmethyl, hydroxylated cyclohexylmethyl, 2-(cyclohexyl)ethyl, (C1-C2)alkyl cyclohexylmethyl, di(C1-C2)alkyl cyclohexylmethyl, hydroxylated (C1-C2)alkyl cyclohexylmethyl, hydroxylated di(C1-C2)alkylcyclohexylmethyl, (3-noradamantyl)methyl, (tetrahydropyranyl)methyl, or oxepanyl methyl;
R⁶ is —H or methyl;
G is NH₂ or NHR^{e};
R^{e} is methyl or R⁵ and R^{e} together are —(CH₂)₃— optionally substituted with C₁-C₄ alkyl or cyclohexyl.

26. The compound of claim 24, wherein:
R⁶ is (C₁-C₇)alkyl, halo(C₁-C₇)alkyl, hydroxy(C₁-C₇)alkyl, cyclohexylmethyl, halocyclohexylmethyl, hydroxylated cyclohexylmethyl, 2-(cyclohexyl)ethyl, (C₁-C₂)alkyl cyclohexylmethyl, di(C₁-C₂)alkyl cyclohexylmethyl, hydroxylated (C₁-C₂)alkyl cyclohexylmethyl, hydroxylated di(C₁-C₂)alkylcyclohexylmethyl, (3-noradamantyl)methyl, (tetrahydropyranyl)methyl, or oxepanyl methyl;
R⁵ is —H or methyl;
G is NH₂ or NHR^{e};
R^{e} is methyl or R⁶ and R^{e} together are —(CH₂)₃— optionally substituted with C₁-C₄ alkyl or cyclohexyl.

27. The compound of claim 25, wherein:
R^{2a} is methyl or ethyl; and
R¹¹ is chloro, fluoro or methyl.

28. The compound of claim 26, wherein:
R^{2a} is methyl or ethyl; and
R¹¹ is chloro, fluoro or methyl.

29. The compound of claim 24, wherein the compound is represented by the following structural formula: 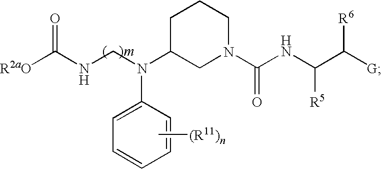
or an enantiomer, diastereomer or a pharmaceutically acceptable salt thereof.

30. The compound of claim 29, wherein the compound is represented by the following structural formulas: 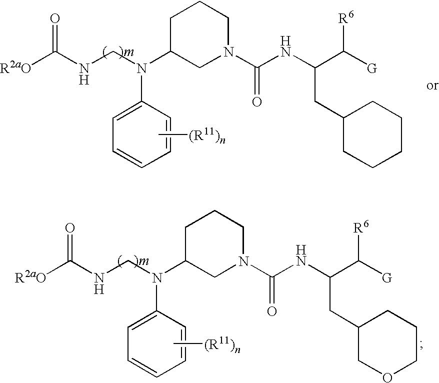
or an enantiomer, diastereomer or a pharmaceutically acceptable salt thereof.

31. A compound of claim 1 selected from: methyl 3-((3-chlorophenyl)(1-(1-cyclohexyl-3-(methylamino)propan-2-ylcarbamoyl)piperidin-3-yl)amino)propylcarbamate, methyl 3-((5-chloro-2-methylphenyl)(1-(1-cyclohexyl-3-(methylamino)propan-2-ylcarbamoyl)piperidin-3-yl)amino)propylcarbamate, methyl 3-((3-chlorophenyl)(1-(1-(methylamino)-3-(-tetrahydro-2H-pyran-3-yl)propan-2-ylcarbamoyl)piperidin-3-yl)amino)propylcarbamate, methyl 3-((3-chlorophenyl)(3-(1-cyclohexyl-3-(methylamino)propan-2-ylcarbamoyl)phenyl)amino)propylcarbamate; or a pharmaceutically acceptable salt thereof.

32. A compound of claim 1 selected from: methyl 3-((3-chlorophenyl)(1-((S)-1-cyclohexyl-3-(methylamino)propan-2-ylcarbamoyl)piperidin-3-yl)amino)propylcarbamate, methyl 3-((5-chloro-2-methylphenyl)((S)-1-((S)-1-cyclohexyl-3-(methylamino)propan-2-ylcarbamoyl)piperidin-3-yl)amino)propylcarbamate, methyl 3-((5-chloro-2-methylphenyl)((R)-1-((S)-1-cyclohexyl-3-(methylamino)propan-2-ylcarbamoyl)piperidin-3-yl)amino)propylcarbamate, methyl 3-((3-chlorophenyl)((R)—((S)-1-(methylamino)-3-((R)-tetrahydro-2H-pyran-3-yl)propan-2-ylcarbamoyl)piperidin-3-yl)amino)propylcarbamate, (S)-methyl 3-((3-chlorophenyl)(3-(1-cyclohexyl-3-(methylamino)propan-2-ylcarbamoyl)phenyl)amino)propylcarbamate or a pharmaceutically acceptable salt thereof.

33. The compound of claim 18, wherein the compound is represented by the following structural formula: 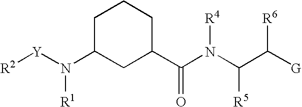
or an enantiomer, diastereomer or a pharmaceutically acceptable salt thereof.

34. The compound of claim 18, wherein the compound is represented by the following structural formula: 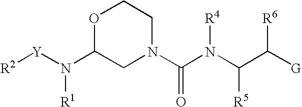
or an enantiomer, diastereomer or a pharmaceutically acceptable salt thereof.

35. A pharmaceutical composition comprising a pharmaceutically acceptable carrier or diluent and the compound of claim 1 or a pharmaceutically acceptable salt thereof.

36. The pharmaceutical composition of claim 35 further comprising a α-blocker, β-blocker, calcium channel blocker, diuretic, natriuretic, saluretic, centrally acting antiphypertensive, angiotensin converting enzyme (ACE) inhibitor, dual ACE and neutral endopeptidase (NEP) inhibitor, angiotensin-receptor blocker (ARB), aldosterone synthase inhibitor, aldosterone-receptor antagonist, or endothelin receptor antagonist.

37.-44. (canceled)
